(19) United States
(12) Patent Application Publication
(10) Pub. No.: US 2016/0331783 A1
(43) Pub. Date: Nov. 17, 2016

(54) BISAMIDE COMPOUNDS AS ALLOSTERIC EFFECTORS FOR REDUCING THE OXYGEN-BINDING AFFINITY OF HEMOGLOBIN

(71) Applicant: THE GENERAL HOSPITAL CORPORATION, Boston, MA (US)

(72) Inventors: Warren M. Zapol, Cambridge, MA (US); Kenneth D. Bloch, Brookline, MA (US); Akito Nakagawa, Cambridge, MA (US); Francine E. Lui, Toronto (CA); Revital Freedman, Brookline, MA (US)

(21) Appl. No.: 15/111,154

(22) PCT Filed: Jan. 13, 2015
(86) PCT No.: PCT/US15/11125

§ 371 Date: Jul. 12, 2016

Related U.S. Application Data
(60) Provisional application No. 61/926,833, filed on Jan. 13, 2014.

(51) Int. Cl. A61K 35/18; C07K 5/062; A61K 38/05; C07K 5/065

(52) U.S. Cl. CPC A61K 35/18; C07K 5/06078; C07K 5/06034; A61K 38/05

(57) ABSTRACT

This invention relates to compounds of Formula (I) and pharmaceutically acceptable salts thereof, which are allosteric effectors that reduce the oxygen-binding affinity of hemoglobin, which can enhance the efficacy of radiation therapy for cancer and which are useful for the treatment of ischemia and other conditions.

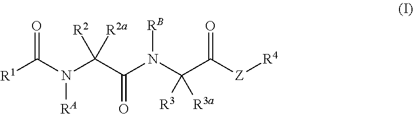

This application claims the benefit of priority of U.S. Provisional Appl. No. 61/926,833, filed Jan. 13, 2014, which is incorporated herein by reference in its entirety.

### TECHNICAL FIELD

This invention relates to bisamide compounds as allosteric effectors that reduce the oxygen-binding affinity of hemoglobin, which enhance the efficacy of radiation therapy for cancer and which are useful for the treatment of ischemia and other conditions.

### BACKGROUND

Hemoglobin is a vital iron-containing metalloprotein in red blood cells (RBCs). Hemoglobin transports oxygen from the lungs to tissues. Oxygen transport of hemoglobin is regulated by two natural allosteric effectors contained in human RBCs; protons and 2,3-diphosphoglycerate (2,3-DPG). These allosteric effectors shift the oxygen binding equilibrium from oxygenated hemoglobin (R-state) to deoxygenated hemoglobin (T-state) by stabilizing the tertiary and quaternary structures of the hemoglobin tetramer. Shifting the equilibrium to the T-state decreases the oxygen-binding affinity of hemoglobin and enhances release of oxygen from hemoglobin.

Organic phosphates, aromatic propionate derivatives, and oligopeptides of tryptophan and phenylalanine derivatives have been studied as allosteric effectors decreasing the oxygen-binding affinity of hemoglobin. These molecules bind to hemoglobin and preferentially stabilize the T-state, shifting the allosteric equilibrium to the T-state which exhibits a reduced oxygen-binding affinity.

Allosteric effectors that reduce the oxygen-binding affinity of hemoglobin may be useful to treat or prevent organ ischemia (such as acute heart attacks and strokes, or chronic organ ischemia such as peripheral vascular insufficiency) and to enhance the efficacy of radiation therapy for cancer by raising the tissue oxygen concentration. For example, red blood cells treated ex vivo with an allosteric effector that reduces the oxygen-binding affinity of hemoglobin and then administered to hamsters with anemia resulted in increased partial oxygen pressure in tissue (Cabrales, P., Tsai, A. G. & Intaglietta, M., “Modulation of perfusion and oxygenation by red blood cell oxygen affinity during acute anemia,” American Journal of Respiratory Cell and Molecular Biology, 38, 354-61 (2008)). Asymptomatic or symptomatic ischemia is the most common manifestation of coronary heart disease in which oxygen delivery to the heart muscle is reduced or blocked (Kilgore, K. S. et al., “RSR13, a synthetic allosteric modifier of hemoglobin, improves myocardial recovery following hypothermic cardiopulmonary bypass,” Circulation 100, 11351-6 (1999)). The oxygen delivery to muscle may be increased at a constant concentration of hemoglobin by decreasing the oxygen binding affinity of hemoglobin so as to release more oxygen within hypoxic tissues (Suh, J. H. et al., “Phase III study of efaproxiral as an adjunct to whole-brain radiation therapy for brain metastases,” Journal of Clinical Oncology, 24, 106-14 (2006)).

A limiting factor to destroying tumors completely by radiotherapy is hypoxia within solid tumors. Hypoxic regions will be minimized if more oxygen is delivered to tumors.

### SUMMARY

The present application provides a composition suitable for administration to an individual, comprising red blood cells (RBCs), treated with a compound of Formula I:

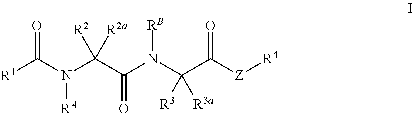

or a pharmaceutically acceptable salt thereof, wherein R¹, R², R^{2a}, R³, R^{3a}, R⁴, Z, R^{A}, and R^{B} are described infra.

The present application also provides a method of treating stored human red blood cells comprising adding a compound of Formula I, or a pharmaceutically acceptable salt thereof, to a composition comprising red blood cells.

The present application further provides a method of enhancing oxygen delivery to a tissue or organ of an individual, comprising administering to said individual a composition described herein, or a compound of Formula I, or a pharmaceutically acceptable salt thereof.

The present application also provides a method of treating acute or chronic ischemia, anemia, coronary infarction, chronic pulmonary disease, congestive heart failure, diabetes, diabetic neuropathy, myocardial infarction, stroke, peripheral vascular disease, peripheral vascular insufficiency, intermittent claudication, circulatory shock, hemorrhagic shock, chronic hypoxia, altitude sickness, arteriosclerosis, respiratory alkalemia, metabolic alkalosis, reduced lung capacity, gangrene, anaerobic infections, carbon monoxide poisoning, nitric oxide poisoning, or cyanide poisoning in an individual in need thereof, comprising administering to said individual a composition described herein or a therapeutically effective amount of a compound of Formula I, or a pharmaceutically acceptable salt thereof.

The present application further provides a method of treating a cancer in an individual in need thereof, comprising administering to said individual a composition of claim 1 or a therapeutically effective amount of compound of Formula I, or a pharmaceutically acceptable salt thereof, in combination with radiation therapy.

In some embodiments, the present application provides a composition described herein or a compound of Formula I, or a pharmaceutically acceptable salt thereof, for use in any of the methods described herein.

In some embodiments, the present application provides use of a composition described herein or a compound of Formula I, or a pharmaceutically acceptable salt thereof, for the preparation of a medicament for use in any of the methods described herein.

### DESCRIPTION OF DRAWINGS

The file of this patent contains at least one drawing executed in color. Copies of this patent with color drawings will be provided by the Patent and Trademark Office upon request and payment of the necessary fee.

FIG. 1 shows an oxygen dissociation curve (ODC) shifted by an allosteric effector by binding to Hb. Right shift of ODC (blue arrow)=decrease of the oxygen binding affinity=increase of P₅₀. Left shift of ODC (red arrow)=increase of the oxygen binding affinity=decrease of P₅₀.

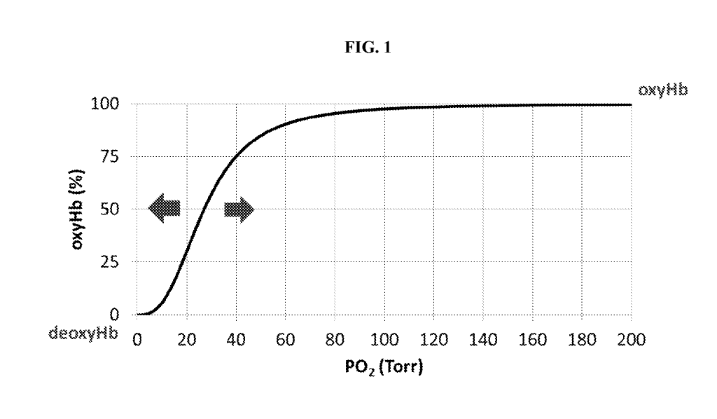

FIG. 2 shows an ODC of hemoglobin (20 μM tetramer) with Compound 1 (200 μM) in Dulbecco's phosphate buffered saline (DPBS, pH 7.4) and 5 vol % DMSO at 37° C. The P₅₀ of hemoglobin with and without Compound 1 was 24 and 17 Torr respectively.

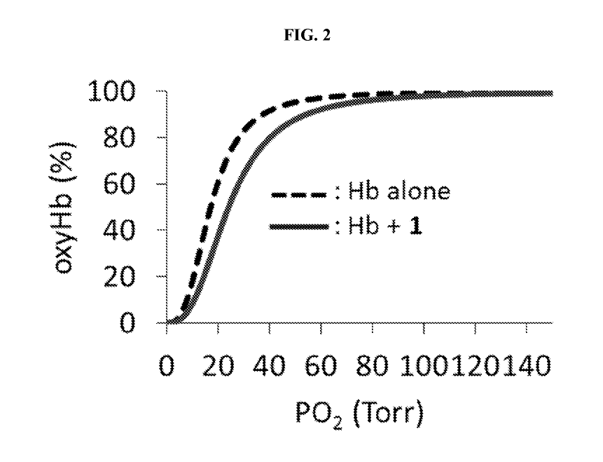

FIG. 3 shows a dose response of P₅₀ of hemoglobin with Compound 1.

FIG. 4 shows an overall tetrameric hemoglobin structure with bound 2,3-diphosphoglycerate (2,3-DPG) and Compound 1 at the dyad axis of the β-cleft (PDB ID: 4L7Y). The protein is displayed in ribbons and/or sticks. Compound 1 and 2,3-DPG are shown in stick format.

FIG. 5 shows a superposition of the β-cleft of deoxyhemoglobin-Compound 1 complex and deoxyhemoglobin without Compound 1 (PDB ID: 2HHB). Compound 1 and 2,3-DPG are shown as sticks. Water molecules are depicted as spheres.

FIG. 6 shows a dose response of P₅₀ of hemoglobin with Compound 1 and the P₅₀ of hemoglobin in the presence of Compound 2.

FIG. 7 shows a dose response of P₅₀ of hemoglobin with Compound 2.

FIG. 8 shows the dose response of P₅₀ in purified human hemoglobin in the presence of Compound 2 and three compounds that enhance oxygen release (2,3-DPG, IHP, and RSR-13).

FIG. 9 shows the P₅₀ of diluted human blood (50 μM) in the presence of Compound 2, RSR-13, IHP, and DPG. Compound/Hb tetramer=30/1 mol/mol. The P₅₀ of blood in the presence of RSR-13 (1.5 mM) could not be evaluated due to hemolysis.

Like reference symbols in the various drawings indicate like elements.

### DETAILED DESCRIPTION

The present application provides, inter alia, compounds which are allosteric effectors that reduce the oxygen-binding affinity of hemoglobin. Such compounds may be useful to treat or prevent organ ischemia and to enhance the efficacy of radiation therapy for cancer by raising the tissue oxygen concentration.

Accordingly, the present application provides a compound of Formula I:

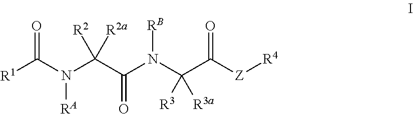

or a pharmaceutically acceptable salt thereof, wherein:

R^{A} is selected from H and C₁₋₃ alkyl;

R^{B} is selected from H and C₁₋₃ alkyl;

R¹ is selected from C₁₋₆ alkyl, C₂₋₆ alkenyl, C₂₋₆ alkynyl, C₁₋₆ haloalkyl, C₃₋₁₀ monocyclic or bicyclic cycloalkyl, 4-10 membered heterocycloalkyl, 6-10 membered aryl, 5-10 membered heteroaryl, C₃₋₁₀ monocyclic or bicyclic cycloalkyl-C₁₋₃ alkylene, 4-10 membered heterocycloalkyl-C₁₋₃ alkylene, 6-10 membered aryl-C₁₋₃ alkylene, and 5-10 membered heteroaryl-C₁₋₃ alkylene, each of which is optionally substituted by 1, 2, 3, or 4 groups independently selected from halo, NO₂, CN, C₁₋₆ alkyl, C₂₋₆ alkenyl, C₂₋₆ alkynyl, C₁₋₆ haloalkyl, Cy¹, —C₁₋₃ alkylene-Cy¹, OR^{a1}, SR^{a1}, C(═O)R^{b1}, C(═O)NR^{c1}R^{d1}, C(═O)OR^{a1}, OC(═O)R^{b1}, OC(═O)NR^{c1}R^{d1}, C(═NR^{e})NR^{c1}R_{d1}, NR^{c1}C(═NR^{e})NR^{c1}R^{d1}, NR^{c1}R^{d1}, NR^{c1}C(═O)R^{b1}, NR^{c1}C(═O)OR^{b1}, NR^{c1}C(═O)NR^{c1}R^{d1}, NR^{c1}S(═O)R^{b}, NR^{c1}S(═O)₂R^{b1}, NR^{c1}S(═O)₂NR^{c1}R^{d1}, S(═O)R^{b1}, S(═O)NR^{c1}R^{d1}, S(═O)₂R^{b1}, and S(═O)₂NR^{c1}R^{d1};

R² is selected from C₁₋₆ alkyl, C₂₋₆ alkenyl, C₂₋₆ alkynyl, C₁₋₆ haloalkyl, Cy, and —C₁₋₃ alkylene-Cy, wherein C₁₋₆ alkyl, C₂₋₆ alkenyl, C₂₋₆ alkynyl, and C₁₋₆ haloalkyl are each optionally substituted by 1, 2, 3, or 4 groups independently selected from CN, OR^{a2}, SR^{a2}, C(═O)R^{b2}, C(═O)NR^{c2}R^{d2}, C(═O)OR^{a2}, OC(═O)R^{b2}, OC(═O)NR^{c2}R^{d2}, C(═NR^{e})NR^{c2}R^{d2}, NR^{c2}C(═NR^{e})NR^{c2}R^{d2}, NR^{c2}R^{d2}, NR^{c2}C(═O)R^{b2}, NR^{c2}C(═O)OR^{b2}, NR^{c2}C(═O)NR^{c2}R^{d2}, NR^{c2}S(═O)R^{b}, NR^{c2}S(═O)₂R^{b2}, NR^{c2}S(═O)₂NR^{c2}R^{d2}, S(═O)R^{b2}, S(═O)NR^{c2}R^{d2}, S(═O)₂R^{b2}, and S(═O)₂NR^{c2}R^{d2};

R^{2a} is selected from H and C₁₋₃ alkyl;

R³ is selected from H, C₁₋₆ alkyl, C₂₋₆ alkenyl, C₂₋₆ alkynyl, C₁₋₆ haloalkyl, C₃₋₁₀ monocyclic or bicyclic cycloalkyl, 4-10 membered heterocycloalkyl, 6-10 membered aryl, 5-10 membered heteroaryl, C₃₋₁₀ monocyclic or bicyclic cycloalkyl-C₁₋₃ alkylene, 4-10 membered heterocycloalkyl-C₁₋₃ alkylene, 6-10 membered aryl-C₁₋₃ alkylene, and 5-10 membered heteroaryl-C₁₋₃ alkylene, wherein said C₁₋₆ alkyl, C₂₋₆ alkenyl, C₂₋₆ alkynyl, C₁₋₆ haloalkyl, C₃₋₁₆ monocyclic or bicyclic cycloalkyl, 4-10 membered heterocycloalkyl, 6-10 membered aryl, 5-10 membered heteroaryl, C₃₋₁₀ monocyclic or bicyclic cycloalkyl-C₁₋₃ alkylene, 4-10 membered heterocycloalkyl-C₁₋₃ alkylene, 6-10 membered aryl-C₁₋₃ alkylene, and 5-10 membered heteroaryl-C₁₋₃ alkylene are each optionally substituted by 1, 2, 3, or 4 independently selected R^{x} groups;

R^{3a} is selected from H and C₁₋₃ alkyl;

Z is selected from O or NR^{C};

R^{C} is H or C₁₋₃ alkyl;

R⁴ is H or C₁₋₃ alkyl;

each Cy is independently selected from C₃₋₁₀ monocyclic or bicyclic cycloalkyl, 4-10 membered heterocycloalkyl, 6-10 membered aryl, and 5-10 membered heteroaryl, each of which is optionally substituted by 1, 2, 3, or 4 substituents independently selected from C₁₋₆ alkyl, C₂₋₆ alkenyl, C₂₋₆ alkynyl, C₁₋₆ haloalkyl, Cy², —C₁₋₃ alkylene-Cy², halo, NO₂, CN, OR^{a2}, SR^{a2}, C(═O)R^{b2}, C(═O)NR^{c2}R^{d2}, C(═O)OR^{a2}, OC(═O)R^{b2}, OC(═O)NR^{c2}R^{d2}, C(═NR^{e})NR^{c2}R^{d2}, NR^{c2}C(═NR^{e})NR^{c2}R^{d2}, NR^{c2}R^{d2}, NR^{c2}C(═O)R^{b2}, NR^{c2}C(═O)OR^{b2}, NR^{c2}C(═O)NR^{c2}R^{d2}, NR^{c2}S(═O)R^{b}, NR^{c2}S(═O)₂R^{b2}, NR^{c2}S(═O)₂NR^{c2}R^{d2}, S(═O)R^{b2}, S(═O)NR^{c2}R^{d2}, S(═O)₂R^{b2}, and S(═O)₂NR^{c2}R^{d2};

each R^{a1}, R^{c1}, and R^{d1} are independently selected from H, C₁₋₆ alkyl, C₂₋₆ alkenyl, C₂₋₆ alkynyl, C₁₋₆ haloalkyl, C₃₋₇ monocyclic cycloalkyl, 4-6 membered heterocycloalkyl, phenyl, 5-6 membered heteroaryl, C₃₋₇ monocyclic cycloalkyl-C₁₋₃ alkylene, 4-6 membered heterocycloalkyl-C₁₋₃ alkylene, phenyl-C₁₋₃ alkylene, and 5-6 membered heteroaryl-C₁₋₃ alkylene; wherein said C₁₋₆ alkyl, C₂₋₆ alkenyl, C₂₋₆ alkynyl, C₁₋₆ haloalkyl, C₃₋₇ monocyclic cycloalkyl, 4-6 membered heterocycloalkyl, phenyl, 5-6 membered heteroaryl, C₃₋₇ monocyclic cycloalkyl-C₁₋₃ alkylene, 4-6 membered heterocycloalkyl-C₁₋₃ alkylene, phenyl-C₁₋₃ alkylene, and 5-6 membered heteroaryl-C₁₋₃ alkylene are each optionally substituted with 1, 2, or 3 independently selected R^{x} groups;

each R^{b1} is independently selected from C₁₋₆ alkyl, C₂₋₆ alkenyl, C₂₋₆ alkynyl, C₁₋₆ haloalkyl, C₃₋₇ monocyclic cycloalkyl, 4-6 membered heterocycloalkyl, phenyl, 5-6 membered heteroaryl, C₃₋₇ monocyclic cycloalkyl-C₁₋₃ alkylene, 4-6 membered heterocycloalkyl-C₁₋₃ alkylene, phenyl-C₁₋₃ alkylene, and 5-6 membered heteroaryl-C₁₋₃ alkylene; each of which are each optionally substituted with 1, 2, or 3 independently selected R^{x} groups;

each R^{a2}, R^{c2}, and R^{d2} are independently selected from H, C₁₋₆ alkyl, C₂₋₆ alkenyl, C₂₋₆ alkynyl, C₁₋₆ haloalkyl, C₃₋₇ monocyclic cycloalkyl, 4-6 membered heterocycloalkyl, phenyl, 5-6 membered heteroaryl, C₃₋₇ monocyclic cycloalkyl-C₁₋₃ alkylene, 4-6 membered heterocycloalkyl-C₁₋₃ alkylene, phenyl-C₁₋₃ alkylene, and 5-6 membered heteroaryl-C₁₋₃ alkylene; wherein said C₁₋₆ alkyl, C₂₋₆ alkenyl, C₂₋₆ alkynyl, C₁₋₆ haloalkyl, C₃₋₇ monocyclic cycloalkyl, 4-6 membered heterocycloalkyl, phenyl, 5-6 membered heteroaryl, C₃₋₇ monocyclic cycloalkyl-C₁₋₃ alkylene, 4-6 membered heterocycloalkyl-C₁₋₃ alkylene, phenyl-C₁₋₃ alkylene, and 5-6 membered heteroaryl-C₁₋₃ alkylene are each optionally substituted with 1, 2, or 3 independently selected R^{x} groups;

each R^{b2} is independently selected from C₁₋₆ alkyl, C₂₋₆ alkenyl, C₂₋₆ alkynyl, C₁₋₆ haloalkyl, C₃₋₇ monocyclic cycloalkyl, 4-6 membered heterocycloalkyl, phenyl, 5-6 membered heteroaryl, C₃₋₇ monocyclic cycloalkyl-C₁₋₃ alkylene, 4-6 membered heterocycloalkyl-C₁₋₃ alkylene, phenyl-C₁₋₃ alkylene, and 5-6 membered heteroaryl-C₁₋₃ alkylene; each of which are each optionally substituted with 1, 2, or 3 independently selected R^{x} groups;

each R^{e} is independently selected from H, CN, OH, C₁₋₄ alkyl, C₁₋₄ alkoxy, NO₂, C(O)(C₁₋₄ alkyl), and S(═O)₂(C₁₋₄ alkyl);

each Cy¹ is independently selected from C₃₋₇ monocyclic cycloalkyl, 4-6 membered heterocycloalkyl, phenyl, and 5-6 membered heteroaryl, each of which is optionally substituted by 1, 2, 3, or 4 independently selected R^{x} groups;

each Cy² is independently selected from C₃₋₁₀ monocyclic or bicyclic cycloalkyl, 4-10 membered heterocycloalkyl, 6-10 membered aryl, 5-10 membered heteroaryl, each of which is optionally substituted by 1, 2, 3, or 4 groups independently selected from C₁₋₆ alkyl, C₂₋₆ alkenyl, C₂₋₆ alkynyl, C₁₋₆ haloalkyl, halo, NO₂, CN, OR^{a2}, SR^{a2}, C(═O)R^{b2}, C(═O)NR^{c2}R^{d2}, C(═O)OR^{a2}, OC(═O)R^{b2}, OC(═O)NR^{c2}R^{d2}, C(═NR^{e})NR^{c2}R^{d2}, NR^{c2}C(═NR^{e})NR^{c2}R^{d2}, NR^{c2}R^{d2}, NR^{c2}C(═O)R^{b2}, NR^{c2}C(═O)OR^{b2}, NR^{c2}C(═O)NR^{c2}R^{d2}, NR^{c2}S(═O)R^{b}, NR^{c2}S(═O)₂R^{b2}, NR^{c2}S(═O)₂NR^{c2}R^{d2}, S(═O)R^{b2}, S(═O)NR^{c2}R^{d2}, S(═O)₂R^{b2}, and S(═O)₂NR^{c2}R^{d2}; and

each R^{x} is independently selected from halo, OH, NO₂, CN, C₁₋₆ alkyl, C₂₋₆ alkenyl, C₂₋₆ alkynyl, C₁₋₆ haloalkyl, cyano-C₁₋₆ alkyl, HO—C₁₋₆ alkyl, C₁₋₆ alkoxy-C₁₋₆ alkyl, C₁₋₆ alkoxy, C₁₋₆ haloalkoxy, amino, C₁₋₆ alkylamino, di(C₁₋₆ alkyl)amino, thio, C₁₋₆ alkylthio, C₁₋₆ alkylsulfinyl, C₁₋₆ alkylsulfonyl, carbamyl, C₁₋₆ alkylcarbamyl, di(C₁₋₆ alkyl)carbamyl, carboxy, C₁₋₆ alkylcarbonyl, C₁₋₆ alkoxycarbonyl, C₁₋₆ alkylcarbonyloxy, C₁₋₆ alkylcarbonylamino, C₁₋₆ alkylsulfonylamino, aminosulfonyl, C₁₋₆ alkylaminosulfonyl, di(C₁₋₆ alkyl)aminosulfonyl, aminosulfonylamino, C₁₋₆ alkylaminosulfonylamino, di(C₁₋₆ alkyl)aminosulfonylamino, aminocarbonylamino, C₁₋₆ alkylaminocarbonylamino, and di(C₁₋₆ alkyl)aminocarbonylamino.

The application further provides a composition suitable for administration to an individual, comprising red blood cells, treated with a compound of Formula I, or a pharmaceutically acceptable salt thereof. In some embodiments, the composition is blood. In some embodiments, the composition comprises stored red blood cells. In some embodiments, the composition comprises red blood cells stored at cold temperatures.

Stored human red blood cells lose natural 2,3-diphosphoglycerate (2,3-DPG) within hours of cold storage. As a result, these red blood cells (RBCs) have a higher oxygen-binding affinity, such that the stored RBCs can fail to unload oxygen as efficiently. By adding the compounds described herein before transfusion to an individual, the oxygen-binding affinity of hemoglobin can be shifted to enable better unloading of oxygen during transfusion.

Accordingly, the present application provides a method of treating stored human red blood cells comprising adding a compound of Formula I, or a pharmaceutically acceptable salt thereof, to a composition comprising red blood cells. In some embodiments, the method further comprises transfusing the composition to an individual. In some embodiment, the method further comprises administering to the individual a compound of Formula I, or a pharmaceutically acceptable salt, following the transfusing of the composition to the individual.

The present application also provides a method of enhancing oxygen delivery to a tissue or organ of an individual, comprising administering to said individual a composition described herein, or a compound of Formula I, or a pharmaceutically acceptable salt thereof.

In some embodiments, the composition, compound, or salt is administered perioperatively. In some embodiments, the composition, compound, or salt is administered during an operation, before an operation, or after an operation.

In some embodiments, the composition, compound, or salt is administered during transfusion, after transfusion, angioplasty, during organ transplant, during treatment for traumatic wound or injury, or in conjunction with treatment with a hyperbaric pressure chamber.

The present application also provides a method of treating acute or chronic ischemia, anemia, coronary infarction, chronic pulmonary disease, congestive heart failure, diabetes, diabetic neuropathy, myocardial infarction, stroke, peripheral vascular disease, peripheral vascular insufficiency, intermittent claudication, circulatory shock, hemorrhagic shock, chronic hypoxia, altitude sickness, arteriosclerosis, respiratory alkalemia, metabolic alkalosis, reduced lung capacity, gangrene, anaerobic infections, carbon monoxide poisoning, nitric oxide poisoning, or cyanide poisoning in an individual in need thereof, comprising administering to said individual a composition of claim 1 or a therapeutically effective amount of a compound of Formula I, or a pharmaceutically acceptable salt thereof.

In some embodiments, the ischemia is associated with peripheral vascular disease, coronary occlusion, cerebral vascular accidents, or tissue transplant.

In some embodiments, the present application further provides a method of treating a cancer in an individual in need thereof, comprising administering to said individual a composition of claim 1 or a therapeutically effective amount of compound of Formula I, or a pharmaceutically acceptable salt thereof, in combination with radiation therapy.

In some embodiments, the cancer is selected from pancreatic cancer, bladder cancer, breast cancer, renal cancer, hepatocellular cancer, lung cancer, ovarian cancer, cervical cancer, gastric cancer, esophageal cancer, head and neck cancer, melanoma, cancer of head and neck, throat cancer, larynx cancer, brain cancer, bone cancer, leukemia, and uterine cancer.

In some embodiments, the cancer is selected from skin cancer, lip cancer, prostate cancer, endometrium cancer, Hodgkin's disease, local extranodal lymphoma, seminoma of testis, dysgerminoma of ovary, medulloblastoma, pineal germinoma, ependymoma, retinoblastoma, choroidal melanoma, Wilms tumor, Rhabdomyosarcoma, colorectal cancer, soft tissue carcinoma, and embryonal carcinoma of testis.

The present application further provides a method of improving exercise capacity in an individual, comprising a composition described herein or a compound of Formula I, or a pharmaceutically acceptable salt thereof, to the individual. For example, in mice, reducing oxygen affinity of hemoglobin has been shown to increase exercise capability (Watanabe T, et al. “Reduction in hemoglobin-oxygen affinity results in the improvement of exercise capacity in mice with chronic heart failure,” J. Am. Coll. Cardiol. 52 (9): 779-86). In some embodiments, the individual suffers from chronic heart failure. In some embodiments, the individual suffers from a disorder or disease described herein.

As used herein, the term “contacting” refers to the bringing together of indicated moieties in an ex vivo or in vivo. For example, “contacting” blood or red blood cells with a compound described herein includes the administration of the compound to an individual or patient, such as a human, as well as, for example, introducing a compound of the invention into a blood composition.

In some embodiments, the contacting comprises contacting said red blood cells of said patient with said compound or salt ex vivo and transfusing the treated red blood cells back to said individual.

As used herein, the term “individual” or “patient,” used interchangeably, refers to any animal, including mammals, preferably mice, rats, other rodents, rabbits, dogs, cats, swine, cattle, sheep, horses, or primates, and most preferably humans.

As used herein, the phrase “therapeutically effective amount” refers to the amount of active compound or pharmaceutical agent that elicits the biological or medicinal response that is being sought in a tissue, system, animal, individual or human by a researcher, veterinarian, medical doctor or other clinician. In some embodiments, the therapeutically effective amount is about 5 mg to about 1000 mg, or about 10 mg to about 500 mg.

As used herein, the term “treating” or “treatment” refers to one or more of (1) preventing the disease; for example, preventing a disease, condition or disorder in an individual who may be predisposed to the disease, condition or disorder but does not yet experience or display the pathology or symptomatology of the disease; (2) inhibiting the disease; for example, inhibiting a disease, condition or disorder in an individual who is experiencing or displaying the pathology or symptomatology of the disease, condition or disorder (i.e., arresting further development of the pathology and/or symptomatology); and (3) ameliorating the disease; for example, ameliorating a disease, condition or disorder in an individual who is experiencing or displaying the pathology or symptomatology of the disease, condition or disorder (i.e., reversing the pathology and/or symptomatology) such as decreasing the severity of disease.

It is further appreciated that certain features of the invention, which are, for clarity, described in the context of separate embodiments, can also be provided in combination in a single embodiment (while the embodiments are intended to be combined as if written in multiply dependent form). Conversely, various features of the invention which are, for brevity, described in the context of a single embodiment, can also be provided separately or in any suitable subcombination.

### Compounds

In some embodiments, R¹ is selected from C₁₋₆ alkyl or phenyl, each of which is optionally substituted by 1, 2, 3, or 4 groups independently selected from halo, NO₂, CN, C₁₋₆ alkyl, C₁₋₆ haloalkyl, OR^{a1}, SR^{a1}, C(═O)R^{b1}, C(═O)NR^{c1}R^{d1}, C(═O)OR^{a1}, OC(═O)R^{b1}, OC(═O)NR^{c1}R^{d1}, C(═NR^{e})NR^{c1}R^{d1}, NR^{c1}C(═NR^{e})NR^{c1}R^{d1}, NR^{c1}R^{d1}, NR^{c1}C(═O)R^{b1}, NR^{c1}C(═O)OR^{b1}, NR^{c1}C(═O)NR^{c1}R^{d1}, NR^{c1}S(═O)R^{b}, NR^{c1}S(═O)₂R^{b1}, NR^{c1}S(═O)₂NR^{c1}R^{d1}, S(═O)R^{b1}, S(═O)NR^{c1}R^{d1}, S(═O)₂R^{b1}, and S(═O)₂NR^{c1}R^{d1}.

In some embodiments, R¹ is selected from C₁₋₆ alkyl and phenyl, wherein said phenyl is optionally substituted with 1, 2, 3, or 4 groups independently selected from C₁₋₄ alkyl. In some embodiments, R¹ is phenyl, which is optionally substituted with 1, 2, 3, or 4 methyl groups.

In some embodiments, R² is selected from C₁₋₆ alkyl and —C₁₋₃ alkylene-Cy. In some embodiments, R² is —C₁₋₃ alkylene-Cy. In some embodiments, Cy is selected from 6-10 membered aryl, which is substituted by Cy².

In some embodiments, each Cy² is phenyl, which is optionally substituted by C₁₋₆ alkyl, C₂₋₆ alkenyl, C₂₋₆ alkynyl, C₁₋₆ haloalkyl, halo, NO₂, CN, OR^{a2}, SR^{a2}, C(═O)R^{b2}, C(═O)NR^{c2}R^{d2}, C(═O)OR^{a2}, OC(═O)R^{b2}, OC(═O)NR^{c2}R^{d2}, C(═NR^{e})NR^{c2}R^{d2}, NR^{c2}C(═NR^{e})NR^{c2}R^{d2}, NR^{c2}R^{d2}, NR^{c2}C(═O)R^{b2}, NR^{c2}C(═O)OR^{b2}, NR^{c2}C(═O)NR^{c2}R^{d2}, NR^{c2}S(═O)R^{b}, NR^{c2}S(═O)₂R^{b2}, NR^{c2}S(═O)₂NR^{c2}R^{d2}, S(═O)NR^{c2}R^{d2}, S(═O)₂R^{b2}, and S(═O)₂NR^{c2}R^{d2}. In some embodiments, each Cy² is phenyl.

In some embodiments, R³ is selected from H, C₁₋₆ alkyl, C₁₋₆ haloalkyl, C₃₋₁₀ monocyclic or bicyclic cycloalkyl-C₁₋₃ alkylene, 4-10 membered heterocycloalkyl-C₁₋₃ alkylene, 6-10 membered aryl-C₁₋₃ alkylene, and 5-10 membered heteroaryl-C₁₋₃ alkylene. In some embodiments, R³ is selected from H, C₁₋₆ alkyl, C₁₋₆ haloalkyl.

In some embodiments, Z is O. In some embodiments, R⁴ is H or methyl. In some embodiments, Z—R⁴ is OH.

In some embodiments, R^{A} is H or methyl.

In some embodiments, R^{B} is H.

In some embodiments, the compound is a compound of Formula II:

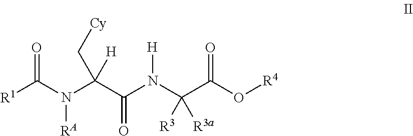

or a pharmaceutically acceptable salt thereof.

In some embodiments, the compound is a compound of Formula III:

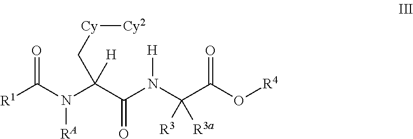

or a pharmaceutically acceptable salt thereof.

In some embodiments:

R¹ is selected from C₁₋₆ alkyl and phenyl, wherein said phenyl is optionally substituted with 1, 2, 3, or 4 groups independently selected from C₁₋₄ alkyl;

R² is selected from C₁₋₆ alkyl and —C₁₋₃ alkylene-Cy;

R³ is selected from H, C₁₋₆ alkyl, C₁₋₆ haloalkyl, C₃₋₁₀ monocyclic or bicyclic cycloalkyl-C₁₋₃ alkylene, 4-10 membered heterocycloalkyl-C₁₋₃ alkylene, 6-10 membered aryl-C₁₋₃ alkylene, and 5-10 membered heteroaryl-C₁₋₃ alkylene;

Z is O;

R⁴ is H or methyl;

R^{A} is H or methyl; and

R^{B} is H.

In some embodiments:

R¹ is selected from C₁₋₆ alkyl and phenyl, wherein said phenyl is optionally substituted with 1, 2, 3, or 4 groups independently selected from C₁₋₄ alkyl;

R² is selected from C₁₋₆ alkyl and —C₁₋₃ alkylene-Cy;

Cy is selected from 6-10 membered aryl, which is substituted by Cy²;

R³ is selected from H, C₁₋₆ alkyl, and C₁₋₆ haloalkyl;

Z is O;

R⁴ is H;

R^{A} is H or methyl; and

R^{B} is H.

In some embodiments:

R¹ is selected from C₁₋₆ alkyl and phenyl, wherein said phenyl is optionally substituted with 1, 2, 3, or 4 groups independently selected from C₁₋₄ alkyl;

R² is selected from C₁₋₆ alkyl and —C₁₋₃ alkylene-Cy;

Cy is selected from 6-10 membered aryl, which is substituted by Cy²;

Cy² is phenyl, which is optionally substituted by C₁₋₆ alkyl, C₂₋₆ alkenyl, C₂₋₆ alkynyl, C₁₋₆ haloalkyl, halo, NO₂, CN, OR^{a2}, SR^{a2}, C(═O)R^{b2}, C(═O)NR^{c2}R^{d2}, C(═O)OR^{a2}, OC(═O)R^{b2}, OC(═O)NR^{c2}R^{d2}, C(═NR^{e})NR^{c2}R^{d2}, NR^{c2}C(═NR^{e})NR^{c2}R^{d2}, NR^{c2}R^{d2}, NR^{c2}C(═O)R^{b2}, NR^{c2}C(═O)OR^{b2}, NR^{c2}C(═O)NR^{c2}R^{d2}, NR^{c2}S(═O)R^{b}, NR^{c2}S(═O)₂R^{b2}, NR^{c2}S(═O)₂NR^{c2}R^{d2}, S(═O)R^{b2}, S(═O)NR^{c2}R^{d2}, S(═O)₂R^{b2}, and S(═O)₂NR^{c2}R^{d2};

R³ is selected from H, C₁₋₆ alkyl, and C₁₋₆ haloalkyl;

Z is O;

R⁴ is H;

R^{A} is H or methyl; and

R^{B} is H.

In some embodiments:

R¹ is selected from C₁₋₆ alkyl and phenyl, wherein said phenyl is optionally substituted with 1, 2, 3, or 4 groups independently selected from C₁₋₄ alkyl;

R² is selected from C₁₋₆ alkyl and —C₁₋₃ alkylene-Cy;

Cy is selected from 6-10 membered aryl, which is substituted by Cy²;

Cy² is phenyl;

R³ is selected from H, C₁₋₆ alkyl, and C₁₋₆ haloalkyl;

Z is O;

R⁴ is H;

R^{A} is H or methyl; and

R^{B} is H.

In some embodiments, the compound is selected from:
(S)-2-((R)-3-([1,1′-biphenyl]-4-yl)-2-(N,3,5-trimethylbenzamido)propanamido)-3-(1H-indol-3-yl)propanoic acid;
(S)-3-(1H-indol-3-yl)-2-((R)-3-phenyl-2-(N,3,5-trimethylbenzamido)propanamido)propanoic acid;
(S)-3-(1H-indol-3-yl)-2-((R)-2-(N,3,5-trimethylbenzamido)butanamido)propanoic acid;
(S)-2-((R)-3-([1,1′-biphenyl]-4-yl)-2-(N-methylacetamido)propanamido)-3-(1H-indol-3-yl)propanoic acid;
(R)-3-(1H-indol-3-yl)-2-((S)-2-(N-methylacetamido)-3-phenylpropanamido)propanoic acid;
(R)-3-(1H-indol-3-yl)-2-((R)-2-(N-methylacetamido)butanamido)propanoic acid;
(S)-methyl 2-((R)-3-([1,1′-biphenyl]-4-yl)-2-(N,3,5-trimethylbenzamido)propanamido)-3-(1H-indol-3-yl)propanoate;
2-(3-([1,1′-biphenyl]-4-yl)-2-(3,5-dimethylbenzamido)propanamido)acetic acid;
2-(3-([1,1′-biphenyl]-4-yl)-2-(N,3,5-trimethylbenzamido)propanamido)acetic acid; and
2-(3-([1,1′-biphenyl]-4-yl)-2-(N,3,5-trimethylbenzamido)propanamido)-2-methylpropanoic acid;
   or a pharmaceutically acceptable salt thereof.

At various places in the present specification, substituents of compounds described herein are disclosed in groups or in ranges. It is specifically intended that the invention include each and every individual subcombination of the members of such groups and ranges. For example, the term “C₁₋₆ alkyl” is specifically intended to individually disclose methyl, ethyl, C₃ alkyl, C₄ alkyl, C₅ alkyl, and C₆ alkyl.

At various places in the present specification, linking substituents are described. Where the structure clearly requires a linking group, the Markush variables listed for that group are understood to be linking groups. For example, if the structure requires a linking group and the Markush group definition for that variable lists “alkyl” or “aryl” then it is to be understood that the “alkyl” or “aryl” represents a linking alkylene group or arylene group, respectively.

At various places in the present specification, rings are described. Unless otherwise specified, these rings can be attached to the rest of the molecule at any ring member as permitted by valency. For example, the term “a 2H-tetrahydropyran ring” may refer to a 2H-tetrahydropyran-2-yl, 2H-tetrahydropyran-3-yl, 2H-tetrahydropyran-4-yl ring, etc.

The term “n-membered” where n is an integer typically describes the number of ring-forming atoms in a moiety where the number of ring-forming atoms is n. For example, 2H-tetrahydropyran is an example of a 6-membered heterocycloalkyl ring, 1H-1,2,4-triazole is an example of a 5-membered heteroaryl ring, pyridine is an example of a 6-membered heteroaryl ring, and 1,2,3,4-tetrahydro-naphthalene is an example of a 10-membered cycloalkyl group.

For compounds in which a variable appears more than once, each variable can be a different moiety independently selected from the group defining the variable. For example, where a structure is described having two R groups that are simultaneously present on the same compound, the two R groups can represent different moieties independently selected from the group defined for R. In another example, when an optionally multiple substituent is designated in the form:

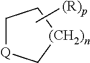

then it is to be understood that substituent R can occur p number of times on the ring, and

R can be a different moiety at each occurrence. It is to be understood that each R group may replace any hydrogen atom attached to a ring atom, including one or both of the (CH₂)ₙ hydrogen atoms. Further, in the above example, should the variable Q be defined to include hydrogens, such as when Q is said to be CH₂, NH, etc., any floating substituent such as R in the above example, can replace a hydrogen of the Q variable as well as a hydrogen in any other non-variable component of the ring.

As used herein, the phrase “optionally substituted” means unsubstituted or substituted. As used herein, the term “substituted” means that a hydrogen atom is removed and replaced by a substituent. It is to be understood that substitution at a given atom is limited by valency.

As used herein, the term “Cₙ₋ₘ alkyl”, employed alone or in combination with other terms, refers to a saturated hydrocarbon group that may be straight-chain or branched, having n to m carbon atoms. In some embodiments, the alkyl group contains 1 to 6, 1 to 4 or 1 to 3 carbon atoms. Examples of alkyl moieties include, but are not limited to, chemical groups such as methyl, ethyl, n-propyl, isopropyl, n-butyl, isobutyl, sec-butyl, tert-butyl, n-pentyl, 2-methyl-1-butyl, 3-pentyl, n-hexyl, 1,2,2-trimethylpropyl, and the like.

As used herein, the term “alkylene”, employed alone or in combination with other terms, refers to a divalent alkyl linking group, which can be branched or straight-chain, where the two substituents may be attached any position of the alkylene linking group. Examples of alkylene groups include, but are not limited to, ethan-1,2-diyl, propan-1,3-diyl, propan-1,2-diyl, butan-1,4-diyl, butan-1,3-diyl, butan-1,2-diyl, 2-methyl-propan-1,3-diyl, and the like.

As used herein, “Cₙ₋ₘ alkenyl” refers to an alkyl group having one or more double carbon-carbon bonds and having n to m carbons. In some embodiments, the alkenyl moiety contains 2 to 6, 2 to 4, or 2 to 3 carbon atoms. Example alkenyl groups include, but are not limited to, ethenyl, n-propenyl, isopropenyl, n-butenyl, sec-butenyl, and the like.

As used herein, “Cₙ₋ₘ alkynyl” refers to an alkyl group having one or more triple carbon-carbon bonds and having n to m carbons. Example alkynyl groups include, but are not limited to, ethynyl, propyn-1-yl, propyn-2-yl, and the like. In some embodiments, the alkynyl moiety contains 2 to 6, 2 to 4, or 2 to 3 carbon atoms.

As used herein, the term “Cₙ₋ₘ alkoxy”, employed alone or in combination with other terms, refers to a group of formula —O-alkyl, wherein the alkyl group has n to m carbons. In some embodiments, the alkyl group contains 1 to 6, 1 to 4 or 1 to 3 carbon atoms. Example alkoxy groups include methoxy, ethoxy, and propoxy (e.g., n-propoxy and isopropoxy).

As used herein, the term “amino” refers to a group of formula —NH₂.

As used herein, the term “Cₙ₋ₘ alkylamino” refers to a group of formula —NH(alkyl), wherein the alkyl group n-m carbon atoms. In some embodiments, the alkyl group contains 1 to 6, 1 to 4 or 1 to 3 carbon atoms.

As used herein, the term “di(Cₙ₋ₘ alkyl)amino” refers to a group of formula —N(alkyl)₂, wherein the two alkyl groups each has, independently, n to m carbon atoms. In some embodiments, each alkyl group independently has 1 to 6, 1 to 4 or 1 to 3 carbon atoms.

As used herein, the term “Cₙ₋ₘ alkoxycarbonyl” refers to a group of formula —C(O)O-alkyl, wherein the alkyl group has n to m carbon atoms. In some embodiments, the alkyl group contains 1 to 6, 1 to 4 or 1 to 3 carbon atoms.

As used herein, the term “Cₙ₋ₘ alkylcarbonyl” refers to a group of formula —C(O)— alkyl, wherein the alkyl group has n to m carbon atoms. In some embodiments, the alkyl group contains 1 to 6, 1 to 4 or 1 to 3 carbon atoms.

As used herein, the term “carboxy” refers to a group of formula —C(O)OH.

As used herein, the term “thio” refers to a group of formula —SH.

As used herein, the term “Cₙ₋ₘ alkylthio” refers to a group of formula —S-(alkyl), wherein the alkyl group has n to m carbon atoms. In some embodiments, the alkyl group contains 1 to 6, 1 to 4 or 1 to 3 carbon atoms.

As used herein, the term “Cₙ₋ₘ alkylsulfinyl” refers to a group of formula —S(O)— alkyl, wherein the alkyl group has n to m carbon atoms. In some embodiments, the alkyl group contains 1 to 6, 1 to 4 or 1 to 3 carbon atoms.

As used herein, the term “Cₙ₋ₘ alkylsulfonyl” refers to a group of formula —S(O)₂-alkyl, wherein the alkyl group has n to m carbon atoms. In some embodiments, the alkyl group contains 1 to 6, 1 to 4 or 1 to 3 carbon atoms.

As used herein, the term “Cₙ₋ₘ alkylcarbonyloxy” refers to a group of formula —OC(O)-alkyl, wherein the alkyl group has n to m carbon atoms. In some embodiments, the alkyl group contains 1 to 6, 1 to 4 or 1 to 3 carbon atoms.

As used herein, the term “Cₙ₋ₘ alkylcarbonylamino” refers to a group of formula —NHC(O)-alkyl, wherein the alkyl group has n to m carbon atoms. In some embodiments, the alkyl group contains 1 to 6, 1 to 4 or 1 to 3 carbon atoms.

As used herein, the term “aminocarbonylamino” refers to a group of formula —NHC(O)NH₂.

As used herein, the term “Cₙ₋ₘ alkylaminocarbonylamino,” refers to a group of formula —NHC(O)NH(alkyl), wherein said alkyl has n to m carbon atoms. In some embodiments, the alkyl group contains 1 to 6, 1 to 4 or 1 to 3 carbon atoms. In some embodiments, each alkyl group independently has 1 to 6, 1 to 4 or 1 to 3 carbon atoms.

As used herein, the term “di(Cₙ₋ₘ alkylaminocarbonylamino” refers to a group of formula —NHC(O)N(alkyl)₂, wherein each alkyl independently has n to m carbon atoms. In some embodiments, each alkyl group independently has 1 to 6, 1 to 4 or 1 to 3 carbon atoms.

As used herein, the term “carbamyl” refers to a group of formula —C(O)—NH₂.

As used herein, the term “Cₙ₋ₘ alkylcarbamyl” refers to a group of formula —C(O)—NH(alkyl), wherein the alkyl group has n to m carbon atoms. In some embodiments, the alkyl group contains 1 to 6, 1 to 4 or 1 to 3 carbon atoms.

As used herein, the term “di(Cₙ₋ₘ-alkyl)carbamyl” refers to a group of formula C(O)N(alkyl)₂, wherein the two alkyl groups each has, independently, n to m carbon atoms. In some embodiments, each alkyl group independently has 1 to 6, 1 to 4 or 1 to 3 carbon atoms.

As used herein, the term “Cₙ₋ₘ alkylsulfonylamino” refers to a group of formula —NHS(O)₂-alkyl, wherein said alkyl has n to m carbon atoms. In some embodiments, the alkyl group contains 1 to 6, 1 to 4 or 1 to 3 carbon atoms.

As used herein, the term “aminosulfonyl” refers to a group of formula —S(O)₂NH₂.

As used herein, the term “Cₙ₋ₘ alkylaminosulfonyl” refers to a group of formula —S(O)₂NH(alkyl), wherein said alkyl has n to m carbon atoms. In some embodiments, the alkyl group contains 1 to 6, 1 to 4 or 1 to 3 carbon atoms.

As used herein, the term “di(Cₙ₋ₘ alkyl)aminosulfonyl” refers to a group of formula —S(O)₂N(alkyl)₂, wherein each alkyl independently has n to m carbon atoms. In some embodiments, each alkyl group independently has 1 to 6, 1 to 4 or 1 to 3 carbon atoms.

As used herein, the term “aminosulfonylamino” refers to a group of formula —NHS(O)₂NH₂.

As used herein, the term “Cₙ₋ₘ alkylaminosulfonylamino,” refers to a group of formula NHS(O)₂NH(alkyl), wherein said alkyl has n to m carbon atoms. In some embodiments, the alkyl group contains 1 to 6, 1 to 4 or 1 to 3 carbon atoms.

As used herein, the term “di(Cₙ₋ₘ alkylaminosulfonylamino” refers to a group of formula —NHS(O)₂N(alkyl)₂, wherein each alkyl independently has n to m carbon atoms. In some embodiments, each alkyl group independently has 1 to 6, 1 to 4 or 1 to 3 carbon atoms.

As used herein, the term “HO—Cₙ₋ₘ-alkyl” refers to a group of formula -alkylene-OH, wherein said alkylene group has n to m carbon atoms. In some embodiments, the alkylene group has 1 to 3 carbon atoms.

As used herein, the term “Cₒ₋ₚ alkoxy-Cₙ₋ₘ-alkyl” refers to a group of formula -alkylene-O-alkyl, wherein said alkylene group has n to m carbon atoms and said alkyl group has o to p carbon atoms. In some embodiments, the alkyl and alkylene groups each independently have 1 to 3 carbon atoms.

As used herein, the term “carbonyl”, employed alone or in combination with other terms, refers to a —C(O)— group.

As used herein, “halo” or “halogen”, employed alone or in combination with other terms, includes fluoro, chloro, bromo, and iodo.

As used herein, the term “Cₙ₋ₘ haloalkyl”, employed alone or in combination with other terms, refers to an Cₙ₋ₘ alkyl group having up to {2(n to m)+1} halogen atoms which may either be the same or different. In some embodiments, the halogen atoms are fluoro atoms. In some embodiments, the alkyl group has 1 to 6 or 1 to 4 carbon atoms. Example haloalkyl groups include CF₃, C₂F₅, CHF₂, CCl₃, CHCl₂, C₂Cl₅, and the like. In some embodiments, the haloalkyl group is a fluoroalkyl group.

As used herein, “Cₙ₋ₘ haloalkoxy” refers to a group of formula —O-haloalkyl having n to m carbon atoms. An example haloalkoxy group is OCF₃. In some embodiments, the haloalkoxy group is fluorinated only. In some embodiments, the alkyl group has 1 to 6 or 1 to 4 carbon atoms.

As used herein, the term “cyano-Cₙ₋ₘ alkyl” refers to a Cₙ₋ₘ alkyl substituted by a cyano group. In some embodiments, the alkyl group has 1 to 3 carbon atoms.

As used herein, the appearance of the term “monocyclic” before the name of a moiety indicates that the moiety has a single ring.

As used herein, the term “cycloalkyl”, employed alone or in combination with other terms, refers to a non-aromatic cyclic hydrocarbon moiety, which may optionally contain one or more alkenylene groups as part of the ring structure. Cycloalkyl groups can include mono- or polycyclic (e.g., having 2, 3 or 4 fused, spirocyclic, or bridged rings) ring systems. Also included in the definition of cycloalkyl are moieties that have one or more aromatic rings fused (i.e., having a bond in common with) to the cycloalkyl ring, for example, benzo derivatives of cyclopentane, cyclopentene, cyclohexane, and the like. One or more ring-forming carbon atoms of a cycloalkyl group can be oxidized to form carbonyl linkages. In some embodiments, cycloalkyl is a 3-10 membered cycloalkyl, which is monocyclic or bicyclic. In some embodiments, cycloalkyl is a 3-6 or 3-7 monocyclic cycloalkyl. Exemplary cycloalkyl groups include 1,2,3,4-tetrahydro-naphthalene, cyclopropyl, cyclobutyl, cyclopentyl, cyclohexyl, cycloheptyl, cyclopentenyl, cyclohexenyl, cyclohexadienyl, cycloheptatrienyl, norbornyl, norpinyl, norcarnyl, adamantyl, and the like. In some embodiments, the cycloalkyl group is cyclopropyl, cyclobutyl, cyclopentyl, or cyclohexyl.

As used herein, the term “aryl”, employed alone or in combination with other terms, refers to a monocyclic or polycyclic (e.g., having 2, 3 or 4 fused rings) aromatic hydrocarbon, such as, but not limited to, phenyl, 1-naphthyl, 2-naphthyl, anthracenyl, phenanthrenyl, and the like. In some embodiments, aryl is C₆₋₁₀ aryl. In some embodiments, the aryl group is a naphthalene ring or phenyl ring. In some embodiments, the aryl group is phenyl.

As used herein, the term “heteroaryl”, employed alone or in combination with other terms, refers to a monocyclic or polycyclic (e.g., having 2, 3 or 4 fused rings) aromatic hydrocarbon moiety, having one or more heteroatom ring members selected from nitrogen, sulfur and oxygen. In some embodiments, heteroaryl is a 5-10 membered heteroaryl, which is monocyclic or bicyclic, comprising 1 to 9 carbon atoms and 1, 2, 3, or 4 heteroatom ring members independently selected from nitrogen, sulfur and oxygen. In some embodiments, heteroaryl is a 5-6 membered heteroaryl, which is monocyclic or bicyclic, comprising 1 to 5 carbon atoms and 1, 2, 3, or 4 heteroatom ring members independently selected from nitrogen, sulfur, and oxygen. When the heteroaryl group contains more than one heteroatom ring member, the heteroatoms may be the same or different. Example heteroaryl groups include, but are not limited to, pyridine, pyrimidine, pyrazine, pyridazine, pyrrole, pyrazole, azolyl, oxazole, thiazole, imidazole, furan, thiophene, quinoline, isoquinoline, indole, benzothiophene, benzofuran, benzisoxazole, imidazo[1,2-b]thiazole, purine, or the like.

A five-membered ring heteroaryl is a heteroaryl with a ring having five ring atoms wherein one or more (e.g., 1, 2, or 3) ring atoms are independently selected from N, O, and S. Exemplary five-membered ring heteroaryls are thienyl, furyl, pyrrolyl, imidazolyl, thiazolyl, oxazolyl, pyrazolyl, isothiazolyl, isoxazolyl, 1,2,3-triazolyl, tetrazolyl, 1,2,3-thiadiazolyl, 1,2,3-oxadiazolyl, 1,2,4-triazolyl, 1,2,4-thiadiazolyl, 1,2,4-oxadiazolyl, 1,3,4-triazolyl, 1,3,4-thiadiazolyl, and 1,3,4-oxadiazolyl.

A six-membered ring heteroaryl is a heteroaryl with a ring having six ring atoms wherein one or more (e.g., 1, 2, or 3) ring atoms are independently selected from N, O, and S. Exemplary six-membered ring heteroaryls are pyridyl, pyrazinyl, pyrimidinyl, triazinyl and pyridazinyl.

As used herein, the term “heteroaryl-C₁₋₃-alkylene” refers to a group of formula alkylene-heteroaryl, wherein the alkylene group has 1-3 carbon atoms. In some embodiments, heteroarylalkyl is C₁₋₉ heteroaryl-C₁₋₃ alkyl, wherein the heteroaryl portion is monocyclic or bicyclic and has 1, 2, 3, or 4 heteroatom ring members independently selected from nitrogen, sulfur and oxygen.

As used herein, the term “aryl-C₁₋₃-alkylene” refers to a group of formula alkylene-aryl, wherein the alkylene group has 1-3 carbon atoms.

As used herein, the term “cycloalkyl-C₁₋₃-alkylene” refers to a group of formula alkylene-cycloalkyl, wherein the alkylene group has 1-3 carbon atoms.

As used herein, the term “heterocycloalkyl”, employed alone or in combination with other terms, refers to non-aromatic ring system, which may optionally contain one or more alkenylene groups as part of the ring structure, and which has at least one heteroatom ring member independently selected from nitrogen, sulfur and oxygen. When the heterocycloalkyl groups contains more than one heteroatom, the heteroatoms may be the same or different. Heterocycloalkyl groups can include mono- or polycyclic (e.g., having 2, 3 or 4 fused, spirocyclic, or bridged rings) ring systems. Also included in the definition of heterocycloalkyl are moieties that have one or more aromatic rings fused (i.e., having a bond in common with) to the non-aromatic ring, for example, 1,2,3,4-tetrahydro-quinoline, 2,3-dihydrobenzo[b][1,4]dioxane, and the like. The carbon atoms or heteroatoms in the ring(s) of the heterocycloalkyl group can be oxidized to form a carbonyl, or sulfonyl group (or other oxidized linkage) or a nitrogen atom can be quaternized. In some embodiments, heterocycloalkyl is 5-10 membered heterocycloalkyl, which is monocyclic or bicyclic, comprising 2 to 9 carbon atoms and 1, 2, 3, or 4 heteroatom ring members independently selected from nitrogen, sulfur, and oxygen. Examples of heterocycloalkyl groups include 2,3-dihydrobenzo[b][1,4]dioxane, 1,2,3,4-tetrahydro-quinoline, azetidine, azepane, pyrrolidine, piperidine, piperazine, morpholine, thiomorpholine, pyran, and a 2-oxo-1,3-oxazolidine ring.

As used herein, the term “heterocycloalkyl-C₁₋₃-alkylene” refers to a group of formula -alkylene-heterocycloalkyl, wherein the alkylene group has 1-3 carbon atoms. In some embodiments, heterocycloalkylalkyl is C₂₋₉ heterocycloalkyl-C₁₋₃ alkyl, wherein the heterocycloalkyl portion is monocyclic or bicyclic and has 1, 2, 3, or 4 heteroatom ring members independently selected from nitrogen, sulfur and oxygen.

The compounds described herein can be asymmetric (e.g., having one or more stereocenters). All stereoisomers, such as enantiomers and diastereomers, are intended unless otherwise indicated. Compounds of the present invention that contain asymmetrically substituted carbon atoms can be isolated in optically active or racemic forms. Methods on how to prepare optically active forms from optically inactive starting materials are known in the art, such as by resolution of racemic mixtures or by stereoselective synthesis. Many geometric isomers of olefins, C═N double bonds, and the like can also be present in the compounds described herein, and all such stable isomers are contemplated in the present invention. Cis and trans geometric isomers of the compounds of the present invention are described and may be isolated as a mixture of isomers or as separated isomeric forms.

Resolution of racemic mixtures of compounds can be carried out by any of numerous methods known in the art. An example method includes fractional recrystallizaion using a chiral resolving acid which is an optically active, salt-forming organic acid. Suitable resolving agents for fractional recrystallization methods are, for example, optically active acids, such as the D and L forms of tartaric acid, diacetyltartaric acid, dibenzoyltartaric acid, mandelic acid, malic acid, lactic acid or the various optically active camphorsulfonic acids such as β-camphorsulfonic acid. Other resolving agents suitable for fractional crystallization methods include stereoisomerically pure forms of α-methylbenzylamine (e.g., S and R forms, or diastereomerically pure forms), 2-phenylglycinol, norephedrine, ephedrine, N-methylephedrine, cyclohexylethylamine, 1,2-diaminocyclohexane, and the like.

Resolution of racemic mixtures can also be carried out by elution on a column packed with an optically active resolving agent (e.g., dinitrobenzoylphenylglycine). Suitable elution solvent composition can be determined by one skilled in the art.

The compounds described herein also include tautomeric forms. Tautomeric forms result from the swapping of a single bond with an adjacent double bond together with the concomitant migration of a proton. Tautomeric forms include prototropic tautomers which are isomeric protonation states having the same empirical formula and total charge. Example prototropic tautomers include ketone-enol pairs, amide-imidic acid pairs, lactam-lactim pairs, enamine-imine pairs, and annular forms where a proton can occupy two or more positions of a heterocyclic system, for example, 1H- and 3H-imidazole, 1H-, 2H- and 4H-1,2,4-triazole, 1H- and 2H-isoindole, and 1H- and 2H-pyrazole. Tautomeric forms can be in equilibrium or sterically locked into one form by appropriate substitution.

The compounds described herein can also include all isotopes of atoms occurring in the intermediates or final compounds. Isotopes include those atoms having the same atomic number but different mass numbers. For example, isotopes of hydrogen include tritium and deuterium.

The term, “compound,” as used herein is meant to include all stereoisomers, geometric iosomers, tautomers, and isotopes of the structures depicted.

All compounds, and pharmaceutically acceptable salts thereof, can be found together with other substances such as water and solvents (e.g., hydrates and solvates) or can be isolated.

In some embodiments, the compounds of the invention, or salts thereof, are substantially isolated. By “substantially isolated” is meant that the compound is at least partially or substantially separated from the environment in which it was formed or detected. Partial separation can include, for example, a composition enriched in the compounds of the invention. Substantial separation can include compositions containing at least about 50%, at least about 60%, at least about 70%, at least about 80%, at least about 90%, at least about 95%, at least about 97%, or at least about 99% by weight of the compounds described herein, or salt thereof. Methods for isolating compounds and their salts are routine in the art.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The expressions, “ambient temperature” and “room temperature,” as used herein, are understood in the art, and refer generally to a temperature, e.g. a reaction temperature, that is about the temperature of the room in which the reaction is carried out, for example, a temperature from about 20° C. to about 30° C.

The present invention also includes pharmaceutically acceptable salts of the compounds described herein. As used herein, “pharmaceutically acceptable salts” refers to derivatives of the disclosed compounds wherein the parent compound is modified by converting an existing acid or base moiety to its salt form. Examples of pharmaceutically acceptable salts include, but are not limited to, mineral or organic acid salts of basic residues such as amines; alkali or organic salts of acidic residues such as carboxylic acids; and the like. The pharmaceutically acceptable salts of the present invention include the non-toxic salts of the parent compound formed, for example, from non-toxic inorganic or organic acids. The pharmaceutically acceptable salts of the present invention can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, non-aqueous media like ether, ethyl acetate, alcohols (e.g., methanol, ethanol, iso-propanol, or butanol) or acetonitrile (ACN) are preferred. Lists of suitable salts are found in Remington's Pharmaceutical Sciences, 17th ed., Mack Publishing Company, Easton, Pa., 1985, p. 1418 and Journal of Pharmaceutical Science, 66, 2 (1977), each of which is incorporated herein by reference in its entirety. In some embodiments, the compounds described herein include the N-oxide forms.

### Synthesis

The compounds described herein, including salts thereof, can be prepared using known organic synthesis techniques and can be synthesized according to any of numerous possible synthetic routes, such as those in the Schemes below. The reactions for preparing the compounds described herein can be carried out in suitable solvents which can be readily selected by one of skill in the art of organic synthesis. Suitable solvents can be substantially non-reactive with the starting materials (reactants), the intermediates, or products at the temperatures at which the reactions are carried out, e.g., temperatures which can range from the solvent's freezing temperature to the solvent's boiling temperature. A given reaction can be carried out in one solvent or a mixture of more than one solvent. Depending on the particular reaction step, suitable solvents for a particular reaction step can be selected by the skilled artisan.

Preparation of the compounds described herein can involve the protection and deprotection of various chemical groups. The need for protection and deprotection, and the selection of appropriate protecting groups, can be readily determined by one skilled in the art. The chemistry of protecting groups can be found, for example, in Wuts and Greene, Protective Groups in Organic Synthesis, 4th ed., John Wiley & Sons: New Jersey, (2007), which is incorporated herein by reference in its entirety.

Reactions can be monitored according to any suitable method known in the art. For example, product formation can be monitored by spectroscopic means, such as nuclear magnetic resonance spectroscopy (e.g., ¹H or ¹³C), infrared spectroscopy, spectrophotometry (e.g., UV-visible), mass spectrometry, or by chromatographic methods such as high performance liquid chromatography (HPLC) or thin layer chromatography (TLC).

Procedures for making compounds described herein are provided below with reference to Schemes 1-2. Specific procedures are provided in the Examples section. Optimum reaction conditions and reaction times may vary depending on the particular reactants used. Unless otherwise specified, solvents, temperatures, pressures and other reaction conditions are readily selected by one of ordinary skill in the art. Compounds are named using the “structure to name” function included in ChemDraw® v.12 (Perkin-Elmer).

Typically, reaction progress may be monitored by thin layer chromatography (TLC) or HPLC-MS if desired. Intermediates and products may be purified by chromatography on silica gel, recrystallization, HPLC and/or reverse phase HPLC. In the reactions described below, it may be necessary to protect reactive functional groups (such as hydroxy, amino, thio, or carboxy groups) to avoid their unwanted participation in the reactions. The incorporation of such groups, and the methods required to introduce and remove them are known to those skilled in the art (for example, see Greene, Wuts, Protective Groups in Organic Synthesis. 2nd Ed. (1999)). One or more deprotection steps in the synthetic schemes may be required. The protecting groups depicted in the schemes are used as examples, and may be replaced by other compatible alternative groups. Starting materials used in the following schemes can be purchased or prepared by methods described in the chemical literature, or by adaptations thereof, using methods known by those skilled in the art. The order in which the steps are performed can vary depending on the protecting or functional groups introduced and the reagents and reaction conditions used, but would be apparent to those skilled in the art.

Compounds of Formula Ia may be prepared according to Scheme 1, shown below. Beginning with intermediate 1i, protection of the amine functionality with 2,4-dimethoxybenzaldehyde at reflux, followed by a standard Pd/C hydrogenation affords intermediate 2i (Step 1). Subsequent reductive amination in the presence of NaBH₃CN/CH₂O affords alkylated R^{B}-amine 3i (Step 2). Acidic cleavage of the 2,4-methoxybenzyl group at elevated temperature then generates the free amine intermediate 4i (Step 3). A standard amine protection (e.g., di-tert-butyl dicarbonate (Boc-anhydride)) affords protected amine 5i (Step 4). A standard peptide coupling procedure (e.g., 1-hydroxybenzotrizole (HOBt), 1-ethyl-3-(3-dimethylaminopropyl)carbodiimide and triethylamine (TEA)) of 5i with the appropriate carboxylic acid or ester afford intermediate 6i (Step 5), from which the deprotection under standard acidic conditions yields intermediate 7i (Step 6). Compound 7A may be prepared from intermediate 7i via a standard peptide coupling procedure, (analogous to Step 5) with a saponification/acidification (Step 8), to afford the desired compound of Formula Ia.

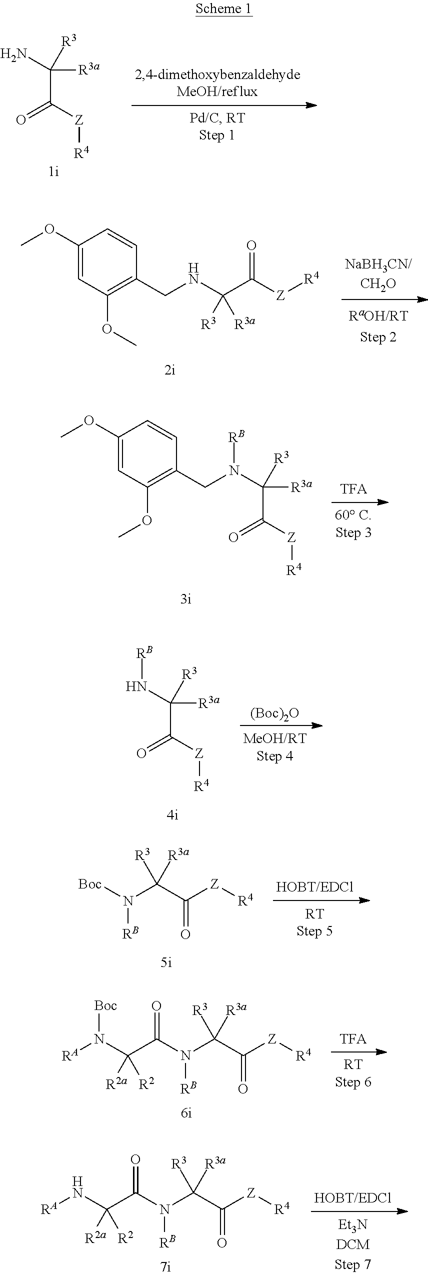

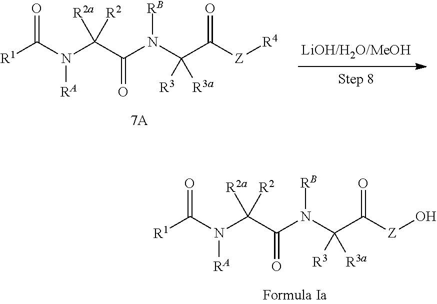

Scheme 2 details the synthesis of compounds of Formula IIa. Beginning with the esterification of (±)-2-amino-3-(4-hydroxyphenyl)propionic acid in the presence of thionyl chloride in methanol to form methyl 2-amino-3-(4-hydroxyphenyl)propanoate (Step 1), the resulting amino-ester may be Boc-protected (Step 2) and reacted with 1,1,1-trifluoro-N-phenyl-N-(trifluoromethylsulfonyl)methanesulfonamide to yield methyl 2-((tert-butoxycarbonyl)amino)-3-(4-(((trifluoromethyl)sulfonyl)oxy)phenyl)propanoate (Step 3). Suzuki coupling with phenylboronic acid in the presence of tetrakis(triphenylphosphine)palladium(0) catalyst (Step 4), followed by removal of the Boc-protecting group under standard acidic conditions (Step 5) affords methyl 3-([1,1′-biphenyl]-4-yl)-2-aminopropanoate, which can be coupled with 3,5-dimethylbenzoic acid (Step 6) to yield methyl 3-([1,1′-biphenyl]-4-yl)-2-(3,5-dimethylbenzamido)propanoate. Subsequent basic hydrolysis and acidification (Step 7), followed by coupling of with the appropriate aminoacetate in the presence of 1-[bis(dimethylamino)methylene]-1H-1,2,3-triazolo[4,5-b]pyridinium 3-oxid hexafluorophosphate (HATU) and N,N-diisopropylethylamine generate the amino-ester intermediate of Step 8. Finally, saponification and acidification may be used to produce the desired compound of Formula IIa (Step 9). The amine functionality of methyl 3-([1,1′-biphenyl]-4-yl)-2-(3,5-dimethylbenzamido)propanoate may also be optionally alkylated using standard alkylation procedures (e.g., reaction with an alkyl halide in the presence of sodium hydride) (Step 6a).

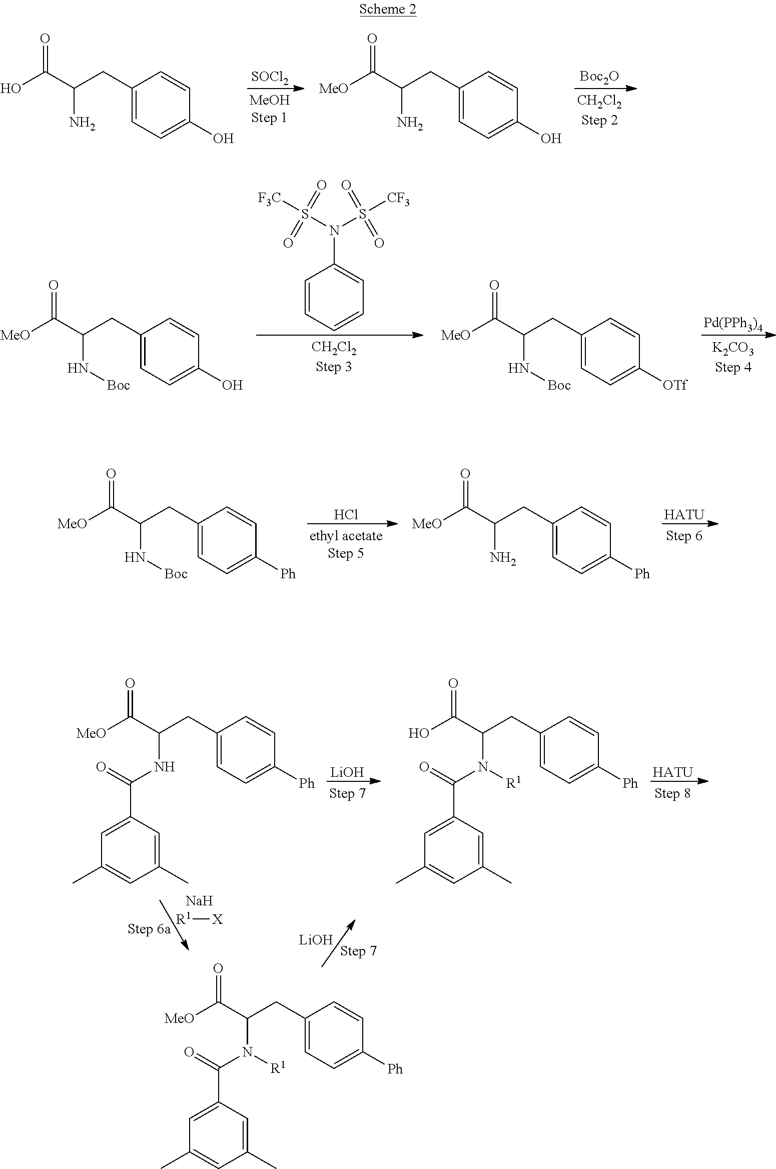

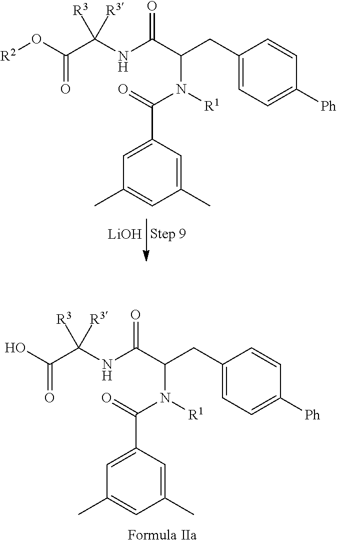

### Pharmaceutical Compositions

When employed as pharmaceuticals, the compounds described herein can be administered in the form of pharmaceutical compositions. These compositions can be prepared in a manner well known in the pharmaceutical art, and can be administered by a variety of routes, depending upon whether local or systemic treatment is desired and upon the area to be treated. Administration may be topical (including transdermal, epidermal, ophthalmic and to mucous membranes including intranasal, vaginal and rectal delivery), pulmonary (e.g., by inhalation or insufflation of powders or aerosols, including by nebulizer; intratracheal or intranasal), sublingual, transcutaneous, intrathecal, oral or parenteral. Parenteral administration includes intravenous, intraarterial, subcutaneous, intraperitoneal intramuscular or injection or infusion; or intracranial, e.g., intrathecal or intraventricular, administration. Parenteral administration can be in the form of a single bolus dose, or may be, for example, by a continuous perfusion pump. Pharmaceutical compositions and formulations for topical administration may include transdermal patches, ointments, lotions, creams, gels, drops, suppositories, sprays, liquids and powders. Conventional pharmaceutical carriers, aqueous, powder or oily bases, thickeners and the like may be necessary or desirable.

This invention also includes pharmaceutical compositions which contain, as the active ingredient, the compound of the invention or a pharmaceutically acceptable salt thereof, in combination with one or more pharmaceutically acceptable carriers (excipients). In some embodiments, the composition is suitable for topical administration. In making the compositions of the invention, the active ingredient is typically mixed with an excipient, diluted by an excipient or enclosed within such a carrier in the form of, for example, a capsule, sachet, paper, or other container. When the excipient serves as a diluent, it can be a solid, semi-solid, or liquid material, which acts as a vehicle, carrier or medium for the active ingredient. Thus, the compositions can be in the form of tablets, pills, powders, lozenges, sachets, cachets, elixirs, suspensions, emulsions, solutions, syrups, aerosols (as a solid or in a liquid medium), ointments containing, for example, up to 10% by weight of the active compound, soft and hard gelatin capsules, suppositories, sterile injectable solutions, and sterile packaged powders.

In preparing a formulation, the active compound can be milled to provide the appropriate particle size prior to combining with the other ingredients. If the active compound is substantially insoluble, it can be milled to a particle size of less than 200 mesh. If the active compound is substantially water soluble, the particle size can be adjusted by milling to provide a substantially uniform distribution in the formulation, e.g. about 40 mesh.

The compounds described herein may be milled using known milling procedures such as wet milling to obtain a particle size appropriate for tablet formation and for other formulation types. Finely divided (nanoparticulate) preparations of the compounds described herein can be prepared by processes known in the art, e.g., see International App. No. WO 2002/000196.

Some examples of suitable excipients include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, syrup, and methyl cellulose. The formulations can additionally include: lubricating agents such as talc, magnesium stearate, and mineral oil; wetting agents; emulsifying and suspending agents; preserving agents such as methyl- and propylhydroxy-benzoates; sweetening agents; and flavoring agents. The compositions of the invention can be formulated so as to provide quick, sustained or delayed release of the active ingredient after administration to the patient by employing procedures known in the art.

In some embodiments, the pharmaceutical composition comprises silicified microcrystalline cellulose (SMCC) and at least one compound described herein, or a pharmaceutically acceptable salt thereof. In some embodiments, the silicified microcrystalline cellulose comprises about 98% microcrystalline cellulose and about 2% silicon dioxide w/w.

In some embodiments, the composition is a sustained release composition comprising at least one compound described herein, or a pharmaceutically acceptable salt thereof, and at least one pharmaceutically acceptable carrier. In some embodiments, the composition comprises at least one compound described herein, or a pharmaceutically acceptable salt thereof, and at least one component selected from microcrystalline cellulose, lactose monohydrate, hydroxypropyl methylcellulose, and polyethylene oxide. In some embodiments, the composition comprises at least one compound described herein, or a pharmaceutically acceptable salt thereof, and microcrystalline cellulose, lactose monohydrate, and hydroxypropyl methylcellulose. In some embodiments, the composition comprises at least one compound described herein, or a pharmaceutically acceptable salt thereof, and microcrystalline cellulose, lactose monohydrate, and polyethylene oxide. In some embodiments, the composition further comprises magnesium stearate or silicon dioxide. In some embodiments, the microcrystalline cellulose is Avicel PH102™. In some embodiments, the lactose monohydrate is Fast-flo 316™. In some embodiments, the hydroxypropyl methylcellulose is hydroxypropyl methylcellulose 2208 K4M (e.g., Methocel K4 M Premier™) and/or hydroxypropyl methylcellulose 2208 K100LV (e.g., Methocel KOOLV™). In some embodiments, the polyethylene oxide is polyethylene oxide WSR 1105 (e.g., Polyox WSR 1105™). In some embodiments, the cyclodextrin is cyclodextrin derivative (e.g., (2-hydroxypropyl)-β-cyclodextrin).

In some embodiments, a wet granulation process is used to produce the composition. In some embodiments, a dry granulation process is used to produce the composition.

The compositions can be formulated in a unit dosage form, each dosage containing from about 5 to about 1,000 mg (1 g), more usually about 100 mg to about 500 mg, of the active ingredient. In some embodiments, each dosage contains about 10 mg of the active ingredient. In some embodiments, each dosage contains about 50 mg of the active ingredient. In some embodiments, each dosage contains about 25 mg of the active ingredient. The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient.

In some embodiments, the compositions of the invention contain from about 5 mg to about 50 mg of the active ingredient. One having ordinary skill in the art will appreciate that this embodies compounds or compositions containing about 5 mg to about 10 mg, about 10 mg to about 15 mg, about 15 mg to about 20 mg, about 20 mg to about 25 mg, about 25 mg to about 30 mg, about 30 mg to about 35 mg, about 35 mg to about 40 mg, about 40 mg to about 45 mg, or about 45 mg to about 50 mg of the active ingredient.

In some embodiments, the compositions of the invention contain from about 50 mg to about 500 mg of the active ingredient. One having ordinary skill in the art will appreciate that this embodies compounds or compositions containing about 50 mg to about 100 mg, about 100 mg to about 150 mg, about 150 mg to about 200 mg, about 200 mg to about 250 mg, about 250 mg to about 300 mg, about 350 mg to about 400 mg, or about 450 mg to about 500 mg of the active ingredient.

In some embodiments, the compositions of the invention contain from about 500 mg to about 1,000 mg of the active ingredient. One having ordinary skill in the art will appreciate that this embodies compounds or compositions containing about 500 mg to about 550 mg, about 550 mg to about 600 mg, about 600 mg to about 650 mg, about 650 mg to about 700 mg, about 700 mg to about 750 mg, about 750 mg to about 800 mg, about 800 mg to about 850 mg, about 850 mg to about 900 mg, about 900 mg to about 950 mg, or about 950 mg to about 1,000 mg of the active ingredient.

The active compound may be effective over a wide dosage range and is generally administered in a pharmaceutically effective amount. It will be understood, however, that the amount of the compound actually administered will usually be determined by a physician, according to the relevant circumstances, including the condition to be treated, the chosen route of administration, the actual compound administered, the age, weight, and response of the individual patient, the severity of the patient's symptoms, and the like.

For preparing solid compositions such as tablets, the principal active ingredient is mixed with a pharmaceutical excipient to form a solid preformulation composition containing a homogeneous mixture of a compound of the present invention. When referring to these preformulation compositions as homogeneous, the active ingredient is typically dispersed evenly throughout the composition so that the composition can be readily subdivided into equally effective unit dosage forms such as tablets, pills and capsules. This solid preformulation is then subdivided into unit dosage forms of the type described above containing from, for example, about 0.1 to about 1000 mg of the active ingredient of the present invention.

The tablets or pills of the present invention can be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer which serves to resist disintegration in the stomach and permit the inner component to pass intact into the duodenum or to be delayed in release. A variety of materials can be used for such enteric layers or coatings, such materials including a number of polymeric acids and mixtures of polymeric acids with such materials as shellac, cetyl alcohol, and cellulose acetate.

The liquid forms in which the compounds and compositions of the present invention can be incorporated for administration orally or by injection include aqueous solutions, suitably flavored syrups, aqueous or oil suspensions, and flavored emulsions with edible oils such as cottonseed oil, sesame oil, coconut oil, or peanut oil, as well as elixirs and similar pharmaceutical vehicles.

Compositions for inhalation or insufflation include solutions and suspensions in pharmaceutically acceptable, aqueous or organic solvents, or mixtures thereof, and powders. The liquid or solid compositions may contain suitable pharmaceutically acceptable excipients as described supra. In some embodiments, the compositions are administered by the oral or nasal respiratory route for local or systemic effect. Compositions in can be nebulized by use of inert gases. Nebulized solutions may be breathed directly from the nebulizing device or the nebulizing device can be attached to a face masks tent, or intermittent positive pressure breathing machine. Solution, suspension, or powder compositions can be administered orally or nasally from devices which deliver the formulation in an appropriate manner.

Topical formulations can contain one or more conventional carriers. In some embodiments, ointments can contain water and one or more hydrophobic carriers selected from, for example, liquid paraffin, polyoxyethylene alkyl ether, propylene glycol, white Vaseline, and the like. Carrier compositions of creams can be based on water in combination with glycerol and one or more other components, e.g. glycerinemonostearate, PEG-glycerinemonostearate and cetylstearyl alcohol. Gels can be formulated using isopropyl alcohol and water, suitably in combination with other components such as, for example, glycerol, hydroxyethyl cellulose, and the like. In some embodiments, topical formulations contain at least about 0.1, at least about 0.25, at least about 0.5, at least about 1, at least about 2, or at least about 5 wt % of the compound of the invention. The topical formulations can be suitably packaged in tubes of, for example, 100 g which are optionally associated with instructions for the treatment of the select indication, e.g., psoriasis or other skin condition.

The amount of compound or composition administered to a patient will vary depending upon what is being administered, the purpose of the administration, such as prophylaxis or therapy, the state of the patient, the manner of administration, and the like. In therapeutic applications, compositions can be administered to a patient already suffering from a disease in an amount sufficient to cure or at least partially arrest the symptoms of the disease and its complications. Effective doses will depend on the disease condition being treated as well as by the judgment of the attending clinician depending upon factors such as the severity of the disease, the age, weight and general condition of the patient, and the like.

The compositions administered to a patient can be in the form of pharmaceutical compositions described above. These compositions can be sterilized by conventional sterilization techniques, or may be sterile filtered. Aqueous solutions can be packaged for use as is, or lyophilized, the lyophilized preparation being combined with a sterile aqueous carrier prior to administration. The pH of the compound preparations typically will be between 3 and 11, more preferably from 5 to 9 and most preferably from 7 to 8. It will be understood that use of certain of the foregoing excipients, carriers, or stabilizers will result in the formation of pharmaceutical salts.

The therapeutic dosage of a compound of the present invention can vary according to, for example, the particular use for which the treatment is made, the manner of administration of the compound, the health and condition of the patient, and the judgment of the prescribing physician. The proportion or concentration of a compound of the invention in a pharmaceutical composition can vary depending upon a number of factors including dosage, chemical characteristics (e.g., hydrophobicity), and the route of administration. For example, the compounds described herein can be provided in an aqueous physiological buffer solution containing about 0.1 to about 10% w/v of the compound for parenteral administration. Some typical dose ranges are from about 1 μg/kg to about 1 g/kg of body weight per day. In some embodiments, the dose range is from about 0.01 mg/kg to about 100 mg/kg of body weight per day. The dosage is likely to depend on such variables as the type and extent of progression of the disease or disorder, the overall health status of the particular patient, the relative biological efficacy of the compound selected, formulation of the excipient, and its route of administration. Effective doses can be extrapolated from dose-response curves derived from in vitro or animal model test systems.

### EXAMPLES

The compounds described herein can be prepared in a number of ways based on the teachings contained herein and synthetic procedures known in the art. In the description of the synthetic methods described below, it is to be understood that all proposed reaction conditions, including choice of solvent, reaction atmosphere, reaction temperature, duration of the experiment and workup procedures, can be chosen to be the conditions standard for that reaction, unless otherwise indicated. It is understood by one skilled in the art of organic synthesis that the functionality present on various portions of the molecule should be compatible with the reagents and reactions proposed. Substituents not compatible with the reaction conditions will be apparent to one skilled in the art, and alternate methods are therefore indicated. The starting materials for the examples are either commercially available or are readily prepared by standard methods from known materials.

¹H- and ¹³C-NMR spectra were acquired at room temperature or 60° C. using a Bruker Avance III (300 MHz for ¹H, 75 MHz for ¹³C). Chemical shifts were referenced to the residual solvent peaks in deuterated DMSO.

High resolution mass-spectrometry (HR-MS) spectra were acquired using a TOF-Agilent 6230 UHPLC/PDA/MS with an ESI source. The difference between the measured ion mass and the expected ion mass was less than 5 ppm.

### Preparation of Synthetic Intermediates

### Intermediate 2i

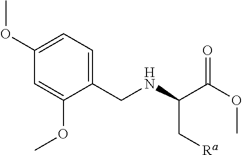

Compound 1i (100 mmol) and 2,4-dimethoxybenzaldehyde (100 mmol) were mixed in MeOH (200 mL) and heated to reflux for 2 h. The resulting mixture was cooled to room temperature followed by addition of Pd/C (5% Pd in C). The suspension was pressurized to 1 bar H₂ and stirred at room temperature for 2 h, at which point the suspension was vented to atmospheric pressure and filtered. The filtrate was then concentrated, resulting in intermediate 2i.

### Intermediate 3i

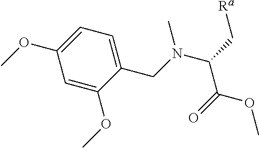

Intermediate 2i (50 mmol) was stirred with NaBH₃CN (50 mmol) dissolved in CH₂O (50 mmol) and MeOH (100 mL) at room temperature and monitored via LC-MS. After 2 h, the reaction mixture was concentrated and the resulting residue was diluted with NaHCO₃ and extracted with dichloromethane (100 mL). The organic layer was collected, dried, and concentrated to yield intermediate 3i.

### Intermediate 4i

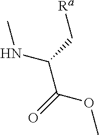

Intermediate 3i (20 mmol) was stirred in trifluoroacetic acid (TFA) (30 mL) and heated to 60° C. for 6 h. The reaction mixture was then cooled and filtered. The resulting filtrate was basified with NaHCO₃ to pH=9 and extracted with ethyl acetate (50 mL×4). The combined organic layers were washed with water (30 mL×2), dried, and concentrated to yield intermediate 4i.

### Intermediate 5i

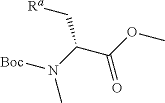

Intermediate 4i (30 mmol) was added to MeOH (100 mL) with (Boc)₂O (30 mmol) and stirred at room temperature. The reaction was monitored via LC-MS. After 2 h, the reaction mixture was concentrated. The residue was purified via chromatography (petroleum ether:ethyl acetate=4:1) to yield intermediate 5i.

### Intermediate 6i

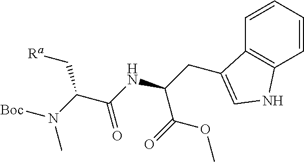

Intermediate 6i (10 mmol) was combined with 1-hydroxybenzotrizole (HOBt) (10 mmol) and 1-ethyl-3-(3-dimethylaminopropyl)carbodiimide (EDCI) (10 mmol) in dichloromethane (100 mL) and stirred at room temperature for 30 min, followed by addition of (S)-methyl 2-amino-3-(1H-indol-3-yl)propanoate (10 mmol) and Et₃N (30 mmol). The mixture was then stirred at room temperature for an additional 3 h. The resulting solution was quenched with NaHCO₃ and the organic layers were separated, dried, concentrated. Intermediate 6i was isolated via chromatography (petroleum ether:ethyl acetate=4:1).

### Intermediate 7i

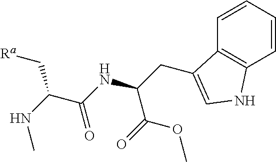

Intermediate 7i (10 mmol) was stirred in trifluoroacetic acid (TFA) (30 mL) at room temperature for 2 h. The resulting mixture was basified with NaHCO₃ to pH=9 and extracted with ethyl acetate (50 mL×4). The combined organic layers were washed with water (30 mL×2), dried, and concentrated to yield intermediate 7i.

### Example Compounds

### Example 1

### Synthesis of a Compound 7A

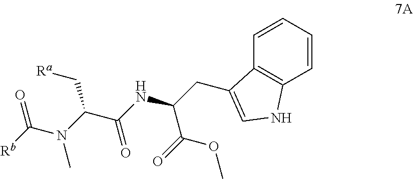

R^{b}COOH (5 mmol), HOBt (5 mmol) and EDCI (5 mmol) were combined in dichloromethane (100 mL) and stirred at room temperature for 30 min, followed by addition of Et₃N and intermediate 7i (5 mmol). The mixture was stirred for an additional 3 h at room temperature. The reaction was then quenched with NaHCO₃ and the organic layers were separated, dried, concentrated. Compounds of 7A were isolated by chromatography (petroleum ether:ethyl acetate=4:1).

### Example 2

### Synthesis of a Compound of Formula Ib

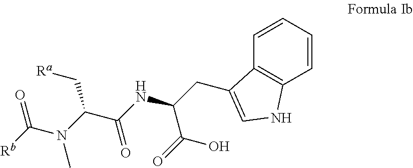

Compound 7A (5 mmol) was added to LiOH (5 mmol) in MeOH/H₂O (30 mL/30 mL) and stirred at room temperature for 6 h. The reaction mixture was acidified with 1M HCl to pH=3 and extracted with ethyl acetate (50 mL×4). The combined organic layers were dried and concentrated to yield a mixture of stereoisomers. The compound of Formula Ib was isolated via HPLC.

### Example 3

### Compounds of Formula Ib

The following compounds (Table 1) were synthesized according to the generic synthesis disclosed in Example 2 using the appropriate starting materials.

TABLE 1
Compounds 1-6
Formula Ib

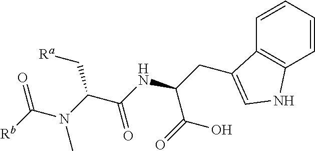

| Compound # | R^{a} | R^{b} | Name | Source |
|---|---|---|---|---|
| 1 | 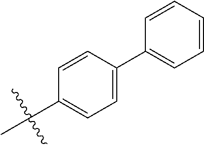 [SMILES: *c1ccc(-c2ccccc2)cc1] | 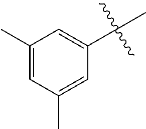 [SMILES: *c1cc(C)cc(C)c1] | (S)-2-((R)-3-([1,1′-biphenyl]-4-yl)-2-(N,3,5- trimethylbenzamido)propanamido)-3-(1H- indol-3-yl)propanoic acid | Santa Cruz Biotechnologies |
| 2 | 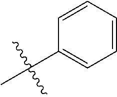 [SMILES: *c1ccccc1] | 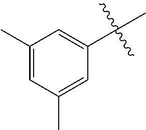 [SMILES: *c1cc(C)cc(C)c1] | (S)-3-(1H-indol-3-yl)-2-((R)-3-phenyl-2- (N,3,5- trimethylbenzamido)propanamido)propanoic acid | Synthesized by Shanghai ChemPartner |
| 3 | Et | 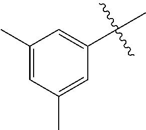 [SMILES: *c1cc(C)cc(C)c1] | (S)-3-(1H-indol-3-yl)-2-((R)-2-(N,3,5- trimethylbenzamido)butanamido)propanoic acid | Synthesized by Shanghai ChemPartner |
| 4 | 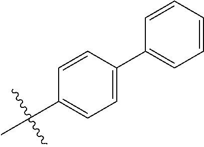 [SMILES: *c1ccc(-c2ccccc2)cc1] | Me | (S)-2-((R)-3-([1,1′-biphenyl]-4-yl)-2-(N- methylacetamido)propanamido)-3-(1H- indol-3-yl)propanoic acid | Synthesized by Shanghai ChemPartner |
| 5 | 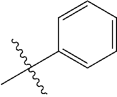 [SMILES: *c1ccccc1] | Me | (R)-3-(1H-indol-3-yl)-2-((S)-2-(N- methylacetamido)-3- phenylpropanamido)propanoic acid | Synthesized by Shanghai ChemPartner |
| 6 | Et | Me | (R)-3-(1H-indol-3-yl)-2-((R)-2-(N- | Synthesized by |
| | | | methylacetamido)butanamido)propanoic | Shanghai |
| | | | acid | ChemPartner |

### Example 4

### (S)-methyl 2-((R)-3-([1,1′-biphenyl]-4-yl)-2-(N,3,5-trimethylbenzamido)propanamido)-3-(1H-indol-3-yl)propanoate (Compound 7)

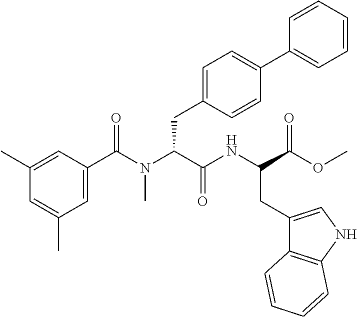

The title compound was prepared according to the procedure of Example 1 using the appropriate starting materials.

### Example 5

### 2-(3-([1,1′-biphenyl]-4-yl)-2-(3,5-dimethylbenzamido)propanamido)acetic acid (Compound 8)

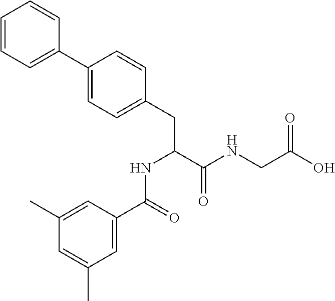 [SMILES: Cc1cc(C)cc(C(=O)NC(Cc2ccc(-c3ccccc3)cc2)C(=O)NCC(=O)O)c1]

### Step 1. Methyl 2-amino-3-(4-hydroxyphenyl)propanoate

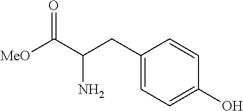 [SMILES: COC(=O)C(N)Cc1ccc(O)cc1]

To a stirred solution of (±)-2-amino-3-(4-hydroxyphenyl)propionic acid (15 g, 0.083 mol) in MeOH (150 mL) was added SOCl₂ (20 g, 0.165 mol) dropwise. The solution was then heated to 60° C. overnight. The resulting solution was then concentrated to give the title compound as a as white solid, which was used without further purification. (16 g, Yield: 100%).

### Step 2. Methyl 2-((tert-butoxycarbonyl)amino)-3-(4-hydroxyphenyl)propanoate

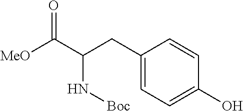 [SMILES: COC(=O)C(Cc1ccc(O)cc1)NC(=O)OC(C)(C)C]

Methyl 2-amino-3-(4-hydroxyphenyl)propanoate (16 g, 0.082 mol) was added to dichloromethane (200 mL) and cooled to 0° C., followed by dropwise addition of Boc₂O (18 g, 0.082 mol). The reaction mixture was warmed to room temperature overnight, washed with brine, and dried over anhydrous magnesium sulfate. The dried mixture was filtered and concentrated to obtain the title compound. (19 g, Yield: 80%).

### Step 3. Methyl 2-((tert-butoxycarbonyl)amino)-3-(4-(((trifluoromethyl)sulfonyl)oxy)phenyl)propanoate

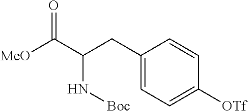 [SMILES: COC(=O)C(Cc1ccc(OS(=O)(=O)C(F)(F)F)cc1)NC(=O)OC(C)(C)C]

To a stirred solution of methyl 2-((tert-butoxycarbonyl)amino)-3-(4-hydroxyphenyl)propanoate (6 g, 0.02 mol) in dichloromethane (100 mL) was added 1,1,1-trifluoro-N-phenyl-N-(trifluoromethylsulfonyl)methanesulfonamide (14.5 g, 0.04 mol) and the mixture was stirred overnight at room temperature. The reaction was then washed with brine and dried over anhydrous magnesium sulfate. The residue was purified by silica gel column chromatography to afford the title compound (6 g, Yield: 70%).

### Step 4. Methyl 3-([1,1′-biphenyl]-4-yl)-2-((tert-butoxycarbonyl)amino)propanoate

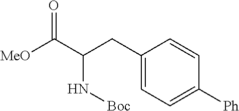 [SMILES: COC(=O)C(Cc1ccc(-c2ccccc2)cc1)NC(=O)OC(C)(C)C]

Nitrogen was passed through a suspension of methyl 2-((tert-butoxycarbonyl)amino)-3-(4-(((trifluoromethypsulfonyl)oxy)phenyl)propanoate (4.27 g, 0.01 mol), phenylboronic acid (2.44 g, 0.02 mol), anhydrous potassium carbonate (2.78 g, 0.02 mol) and toluene (50 mL) for 15 minutes. Tetrakis(triphenylphosphine)palladium(0) (400 mg) was added, and the mixture was heated at 85°-90° for 3 h. The reaction mixture was then cooled to 25°, diluted with ethyl acetate, washed with brine and dried over anhydrous magnesium sulfate. The residue was purified via silica gel column chromatography to obtain the title compound (2.5 g, Yield: 69%).

### Step 5. Methyl 3-([1,1′-biphenyl]-4-yl)-2-aminopropanoate

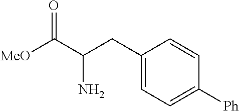 [SMILES: COC(=O)C(N)Cc1ccc(-c2ccccc2)cc1]

Methyl 3-([1,1′-biphenyl]-4-yl)-2-((tert-butoxycarbonyl)amino)propanoate (4 g, 0.011 mol) was dissolved in ethyl acetate/HCl (4N) (30 mL) and the reaction was stirred overnight. The mixture was then filtered to afford the title compound as white solid (3 g, Yield: 100%).

### Step 6. Methyl 3-([1,1′-biphenyl]-4-yl)-2-(3,5-dimethylbenzamido)propanoate

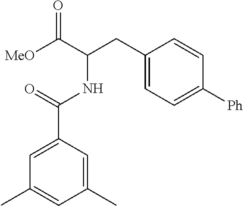 [SMILES: COC(=O)C(Cc1ccc(-c2ccccc2)cc1)NC(=O)c1cc(C)cc(C)c1]

N,N-Diisopropylethylamine (DIPEA) (2.6 g, 0.02 mol), 3,5-dimethylbenzoic acid (3 g, 0.02 mol) and HATU (7.6 g, 0.02 mol) were added to a solution of methyl 3-([1,1′-biphenyl]-4-yl)-2-aminopropanoate (3 g, 0.01 mol) in DMF (20 mL) and allowed to stir overnight at room temperature. The reaction was then diluted with ethyl acetate/H₂O, washed with brine, and the organic phase was dried over anhydrous magnesium sulfate. The residue was filtered and concentrated to obtain the title compound (3.5 g, Yield: 90%).

### Step 7. 3-([1,1′-biphenyl]-4-yl)-2-(3,5-dimethylbenzamido)propanoic acid

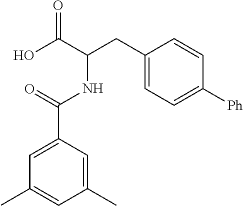 [SMILES: Cc1cc(C)cc(C(=O)NC(Cc2ccc(-c3ccccc3)cc2)C(=O)O)c1]

LiOH (0.21 g, 0.0005 mol) was added to a stirred solution of methyl 3-([1,1′-biphenyl]-4-yl)-2-(3,5-dimethylbenzamido)propanoate (1 g, 0.0025 mol) in MeOH (10 mL), THF (10 mL), and H₂O (10 mL). The mixture was stirred for 2 h, followed by addition of HCl to adjust the pH to 2-3. The mixture was extracted with ethyl acetate, washed with brine, dried, and concentrated to obtain the title compound (0.8 g, Yield: 80%).

### Step 8. Ethyl 2-(3-([1,1′-biphenyl]-4-yl)-2-(3,5-dimethylbenzamido)propanamido)acetate

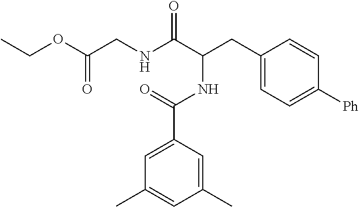 [SMILES: CCOC(=O)CNC(=O)C(Cc1ccc(-c2ccccc2)cc1)NC(=O)c1cc(C)cc(C)c1]

DIPEA (0.52 g, 0.004 mol), ethyl 2-aminoacetate hydrochloride (0.56 g, 0.004 mol) and HATU (1.52 g, 0.004 mol) were added to a stirred solution of 3-([1,1′-biphenyl]-4-yl)-2-(3,5-dimethylbenzamido)propanoic acid (0.8 g, 0.002 mol) in DMF (10 mL), and the mixture was stirred overnight at room temperature. The reaction was then diluted with ethyl acetate/H₂O. The organic phase was washed with brine, dried over anhydrous magnesium sulfate, filtered and concentrated to obtain the title compound (0.6 g, Yield: 60%).

### Step 9. 2-(3-([1,1′-biphenyl]-4-yl)-2-(3,5-dimethylbenzamido)propanamido)acetic acid

LiOH (0.11 g, 0.0026 mol) was added to a stirred solution of ethyl 2-(3-([1,1′-biphenyl]-4-yl)-2-(3,5-dimethylbenzamido)propanamido)acetate (0.6 g, 0.0013 mol) in MeOH (10 mL), THF (10 mL), and water (10 mL) and stirred for 2 h. HCl was then added to adjust to pH=2˜3, and the mixture was extracted with ethyl acetate. The organic phase was washed with brine, dried, and concentrated to obtain the title compound (0.15 g, Yield: 25%).

### Example 6

### 2-(3-([1,1′-biphenyl]-4-yl)-2-(N,3,5-trimethylbenzamido)propanamido)acetic acid (Compound 9)

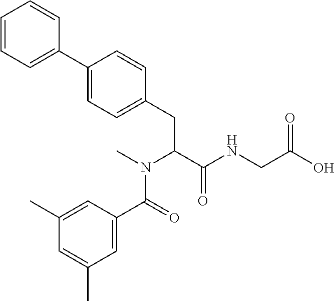 [SMILES: Cc1cc(C)cc(C(=O)N(C)C(Cc2ccc(-c3ccccc3)cc2)C(=O)NCC(=O)O)c1]

### Step 1. methyl 3-([1,1′-biphenyl]-4-yl)-2-(N,3,5-trimethylbenzamido)propanoate

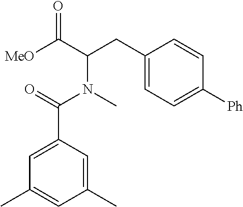 [SMILES: COC(=O)C(Cc1ccc(-c2ccccc2)cc1)N(C)C(=O)c1cc(C)cc(C)c1]

Sodium hydride (0.2 g, 0.005 mol) was added to a stirred solution of methyl 3-([1,1′-biphenyl]-4-yl)-2-(3,5-dimethylbenzamido)propanoate (2 g, 0.005 mol) in DMF 20 (mL), followed by addition of CH₃I (0.71 g, 0.005 mol) and allowed to stir overnight at room temperature. The reaction was diluted with ethyl acetate/H₂O, and the organic phase was washed with brine and dried over anhydrous magnesium sulfate. After drying, the organic phase was filtered and concentrated to obtain the title compound (1.8 g, Yield: 90%).

### Step 2. 3-([1,1′-biphenyl]-4-yl)-2-(N,3,5-trimethylbenzamido)propanoic acid

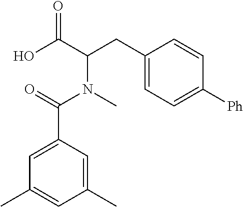 [SMILES: Cc1cc(C)cc(C(=O)N(C)C(Cc2ccc(-c3ccccc3)cc2)C(=O)O)c1]

The title compound was prepared from methyl 3-([1,1′-biphenyl]-4-yl)-2-(N,3,5-trimethylbenzamido)propanoate according to the procedure of Scheme 2, Step 7.

### Step 3. Ethyl 2-(3-([1,1′-biphenyl]-4-yl)-2-(N,3,5-trimethylbenzamido)propanamido)acetate

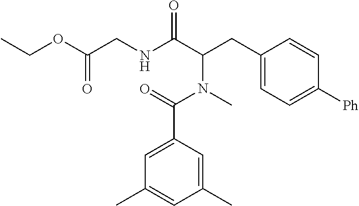 [SMILES: CCOC(=O)CNC(=O)C(Cc1ccc(-c2ccccc2)cc1)N(C)C(=O)c1cc(C)cc(C)c1]

The title compound was prepared via the coupling of 3-([1,1′-biphenyl]-4-yl)-2-(N,3,5-trimethylbenzamido)propanoic acid and ethyl 2-aminoacetate according to the procedure of Scheme 2, Step 8.

### Step 4. 2-(3-([1,1′-biphenyl]-4-yl)-2-(N,3,5-trimethylbenzamido)propanamido)acetic acid

The title compound was prepared from ethyl 2-(3-([1,1′-biphenyl]-4-yl)-2-(N,3,5-trimethylbenzamido)propanamido)acetate according to the procedure of Scheme 2, Step 9.

### Example 7

### 2-(3-([1,1′-biphenyl]-4-yl)-2-(N,3,5-trimethylbenzamido)propanamido)-2-methylpropanoic acid (Compound 10)

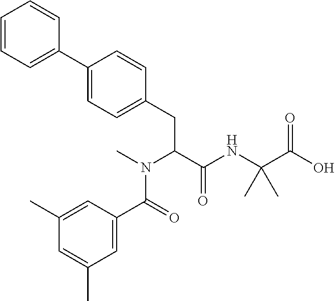 [SMILES: Cc1cc(C)cc(C(=O)N(C)C(Cc2ccc(-c3ccccc3)cc2)C(=O)NC(C)(C)C(=O)O)c1]

### Step 1. methyl 2-(3-([1,1′-biphenyl]-4-yl)-2-(N,3,5-trimethylbenzamido)propanamido)-2-methylpropanoate

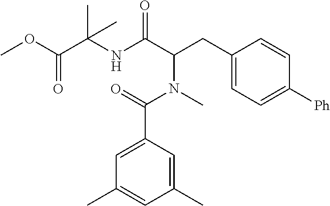 [SMILES: COC(=O)C(C)(C)NC(=O)C(Cc1ccc(-c2ccccc2)cc1)N(C)C(=O)c1cc(C)cc(C)c1]

The title compound was prepared via the coupling of 3-([1,1′-biphenyl]-4-yl)-2-(N,3,5-trimethylbenzamido)propanoic acid and methyl 2-amino-2-methylpropanoate according to the procedure of Scheme 2, Step 8.

### Step 2. 2-(3-([1,1′-biphenyl]-4-yl)-2-(N,3,5-trimethylbenzamido)propanamido)-2-methylpropanoic acid

The title compound was prepared from methyl 2-(3-([1,1′-biphenyl]-4-yl)-2-(N,3,5-trimethylbenzamido)propanamido)-2-methylpropanoate according to the procedure of Scheme 2, Step 9.

### Example 8

### ¹H-NMR Data

TABLE 2
¹H-NMR data for compounds 8-10

| Compound # | ¹H-NMR |
|---|---|
| 8 | DMSO-d₆: δ 12.61 (s, 1H), 8.52, (d, 1H), 8.43 (d, 1H), |
| | 7.60 (dd, 4H), 7.44 (m, 6H), 7.32 (m, 1H), 7.13 (s, 1H), |
| | 4.80 (p, 1H), 3.82 (t, 2H), 3.16 (dd, 1H), 3.04 (t, 1H), |
| | 2.29 (s, 6H). |
| 9 | DMSO-d₆: δ 12.64 (s, 1H), 8.40 (d, 1H), 7.60 (m, 4H), |
| | 7.40 (m, 4H), 7.09 (d, 1H), 6.95 (dd, 1H), 6.54 (s, 1H), |
| | 6.38 (s, 1H), 4.40 (d, 1H), 3.81 (m, 2H), 3.33 (m, 3H), |
| | 2.99 (m, 3H), 2.73 (s, 1H), 2.14 (d, 6H). |
| 10 | DMSO-d₆: δ 12.33 (s, 1H), 8.18 (s, 1H), 7.64 (m, 4H), |
| | 7.42 (m, 4H), 7.16 (d, 1H), 7.00 (s, 1H), 6.51 (d, 2H), 5.45 |
| | (m, 1H), 4.41 (m, 1H), 3.04 (m, 4H), 2.75 (d, 2H), 2.19 |
| | (s, 6H), 1.4 (s, 6H). |

### Example 9

### Preparation of Hemoglobin

Outdated donated human red blood cells (RBCs) were obtained from the Blood Bank of Massachusetts General Hospital. The RBCs were centrifuged at 3,000 rpm for 15 min at 4° C. The supernatant was discarded, and the remaining RBCs were washed three times with an equal volume of 0.9% sodium chloride. Three equivalents of distilled water was added to the washed RBCs, and the mixture was centrifuged at 20,000 rpm for 1 h at 4° C. The supernatant was collected and centrifuged again under the same conditions. The resulting supernatant was collected and dialyzed against Dulbecco's phosphate buffered saline (DPBS, pH 7.4) at 4° C. After dialysis, the sample was sterilized by twice passing it through 0.2 μm filters to confirm that the solution would pass through smoothly.

The absorption spectrum of the hemoglobin solution was measured from 500-700 nm, and the absorption spectra were fitted to a linear combination of pure oxyHb, deoxyHb and metHb plus a baseline (least-squared fitting) using a program (Solver in Excel 2007, Microsoft) to determine the total concentration of hemoglobin.

### Example 10

### Identification of Small Molecules Binding to Hemoglobin Using SMMs

Test compounds were affixed to SMM slides with isocyanate-functionalized surfaces as described in Casalena et al., Methods in molecular biology, 2012, 803, 249-263. Pre-incubating the SMMs with albumin blocked non-specific binding of the compounds to SMMs. Compounds binding to hemoglobin were detected on the SMMs using a fluorescent dye-conjugated antibodies specific for hemoglobin. The assay was validated by confirming the binding of hemoglobin to 2,3-DPG affixed on SMMs.

Each slide contained 48 “blocks”, and each block contained approximately 100 unique surface-immobilized small molecules. Compounds were printed in duplicate, and each slide was screened in duplicate, resulting in four replicates for every test compound. SMM slides were blocked by incubating them in a TBS-T solution (Tween 20, 0.01 vol %) containing bovine serum albumin (BSA, 0.1 wt %) for 30 min. The slides were rinsed with TBS-T buffer for 2 min and incubated in a TBS-T solution containing purified hemoglobin (1 μg/mL). The hemoglobin-treated slides were rinsed with TBS-T buffer for 2 min and incubated with a TBS-T solution containing an antibody directed against human hemoglobin (mouse-IgG, 0.25 μg/mL Catalog No. ab55081, Abcam) for 30 min. Antibody-treated slides were washed for 2 min with TBS-T buffer and then incubated with a secondary detection anti-mouse IgG antibody labeled with Cy5 fluorescent dye (0.2 μg/mL, Catalog No. A10524, Invitrogen) for 30 min, followed by washing three times with TBS-T and once with distilled water, and 2 min on a shaker. All incubations were performed at room temperature and on a shaker set at the lowest setting. Slides were scanned using GenePix 4200A (Molecular Devices) with excitation wavelengths of 635 nm.

Using SMM Hits-Analyzer software (Broad Institute), the measured fluorescent signal of the foreground of a given spot (F) was divided by its local background (B) resulting in a calculated F/B ratio for each spot on which the test compounds were affixed. The F/B ratios were fitted to a Cauchy distribution plot for every block on the slide, and “positive” hits were determined using a p-value threshold of 7%. If all four “positive” replicates had p-values below 7%, they were selected as “hit” compounds binding to hemoglobin. An antibody counter screen was performed to exclude false positives that could be binding antibodies used in the testing, rather than the hemoglobin target.

### Example 11

### Screening of Small Molecules Altering Oxygen Binding Affinity of Hemoglobin

### Sample Preparation for Hemoglobin Oxygen Binding Assay

Test compounds (0.75 μL, 10 mM in DMSO) were added to the central area of two 384 deep well plates forming compound plates. DPBS was used as the solvent for myo-inositol hexaphosphate (IHP) and N-ethylmaleimide (NEM) (10 mM). To prevent oxidation of hemoglobin during the experiment, glucose-6-phosphate (0.4 mg/mL), glucose-6-phosphate dehydrogenase (1.0 U/mL), nicotinamide adenine dinucleotide phosphate (NADP, 0.05 mg/mL), ferredoxin (0.01 mg/mL), ferredoxin-NADP reductase (0.01 U/mL) and catalase (2×10³ U/mL) were added to hemoglobin to produce the Hayashi reducing system, which is described in detail in Hayashi et al., Biochemica et Biophysica Acta, 1973, 310, 309-316.

The DPBS solution containing hemoglobin (10 μM) with the Hayashi reducing system (125 μL/well) was dispensed into the test compound plates, and the plates were shaken at 800 rpm for 3 min and centrifuged at 1,000 rpm for 1 min. The mixture of hemoglobin with each compound (50 μL/well) in the compound plates was transferred to four 384 well plates serving as the assay plates. The final concentration of hemoglobin, test compound and DMSO was 10 μM, 60 μM and 5 vol %, respectively.

### Design of Hemoglobin Oxygen-Binding Assay

The alteration of hemoglobin's oxygen-binding affinity by the test compounds binding to hemoglobin was evaluated in an environment where the fractional oxygen pressure (FO₂) could be precisely measured and controlled. The assay was validated by measuring oxyHb % with and without two known allosteric effectors, myo-inositol hexaphosphate (IHP) and N-ethylmaleimide (NEM), at an allosteric effector to hemoglobin tetramer concentration of 6 to 1 (mol/mol). The IHP-induced decrease and NEM-induced increase of oxyHb % were consistent with their known ability to shift oxygen dissociation curves (ODCs) of hemoglobin. To confirm the allosteric effect of the test compounds, ODC of hemoglobin was measured in the presence and absence of each compound. The value of P₅₀ (the oxygen pressure at which the oxyHb level is 50%) was evaluated as a parameter of oxygen binding affinity.

The ability of small molecules to alter the oxygen affinity of hemoglobin was determined by measuring the fraction of oxygenated hemoglobin (oxyHb %) and oxidized hemoglobin (metHb %) using spectrophotometry under three (high, medium, and low oxyHb saturation) conditions. Hemoglobin was mixed with test compounds, and the mixture was added to 240 central wells of 384-well plates (50 μL/well). The concentrations of hemoglobin, compound, and dimethyl sulfoxide (DMSO) were 10 μM, 60 μM, and 5 vol % in Dulbecco's phosphate buffered saline (DPBS), respectively.

To control the environment, a plate reader (MultiSkan GO, Thermo Fisher Scientific), a plate shaker, thermometer, and a calibrated oxygen pressure meter were placed in a chamber (AtmosBag, Sigma). The FO₂ in the chamber was controlled by purging and then continuously providing a precise mixture of nitrogen and air into the chamber and was monitored via the oxygen pressure meter. Gas cylinders of nitrogen gas and air were connected to a gas proportioner to adjust the flow rate of nitrogen gas and air to the chamber. The gas proportioner was connected to a humidifier to maintain 70-85% humidity within the chamber, and the humidifier was connected to the gas inlet of the chamber. A gas outlet allowed gas release at atmospheric pressure.

The developed oxygen binding assay allows for a large number of assays to be conducted simultaneously with only small quantities of test molecules (12 nmoles per well).

### Measurement of the Alteration of Oxygen-Binding Affinity Using the Hemoglobin-Oxygen Binding Assay

Assay plates were transferred into the aforementioned chamber and equilibrated at three stepwise descending oxygen pressures at 25-27° C. For the highest saturation condition of oxygenated hemoglobin, the assay plates were incubated without shaking at FO_{2=1.2}% for 12 h, and visible absorption spectra of the samples were measured from 500 nm to 700 nm at 2 nm steps using the plate reader. After the measurement of the absorption spectra, the test plates were shaken for 1 h at FO_{2=1.2}% and the spectra were re-measured to obtain the medium oxygen saturation condition. The FO₂ was then reduced to 0.5% and the plates were shaken for 1 h. The spectra were then measured to obtain the lowest saturation condition. To determine the fraction of oxyhemoglobin saturation (oxyHb %) of each sample, the measured absorption spectra were analyzed by least-squared fitting to determine oxyHb % and metHb % using Solver (Microsoft Excel 2007).

### Statistical Analysis

In the hemoglobin-oxygen binding assay, an uneven distribution of oxyHb % between the wells of each plate was observed when oxyHb % without compounds was measured. To account for the uneven oxygenation distribution, a calibration factor was introduced for each well. To determine the calibration factor, hemoglobin was added to the central 240 wells, and the plate was replicated to four plates. Absorption spectra were measured at the assay saturation conditions and oxyHb % was determined by the least-squared fitting. The calibration factor was determined for each of the 240 wells at all three saturation conditions (highest, medium and lowest oxyHb %). For the selection of compounds altering oxyHb saturation, the value of oxyHb % was defined as the sum of oxyHb % calculated by the spectral deconvolution and the calibration factor.

The Z score for oxyHb % containing a unique compound (Z_{oxy}) was calculated according to Equation 1. In Equation 1, oxyHb %_{cpd} is oxyHb % containing a unique compound, and oxyHb %_{ctrl} is oxyHb % without any compound.

Equation 1. Z-Score for oxyHb % containing a unique compound

Z_{oxy}=(Mean value of oxyHb%_{cpd}−Mean value of oxyHb%_{ctrl})/Standard deviation of oxyHb%_{ctrl}

The Z score was also calculated for metHb (Zₘₑₜ) according to Equation 2. In Equation 2, metHb %_{cpd} is metHb % containing a unique compound, and metHb %_{cpd} is metHb % without any added compound. Compounds with (1) both Z_{oxy}>2.5 (increase of oxyHb %) and Zₘₑₜ<2.5 or (2) both Z_{oxy}<−2.5 (decrease of oxyHb %) and Zₘₑₜ<2.5 were selected as hit compounds that alter oxyHb % without increasing metHb formation.

Equation 2. Z-Score for metHb(%)

Zₘₑₜ=(Mean value of metHb(%)_{cpd}−Mean value of metHb(%)_{ctrl})/Standard deviation of MetHb(%)_{ctrl}

### Example 12

### Measurement of Oxygen Dissociation Curve (ODC)

The ODC of hemoglobin was measured with a Hemox analyzer (TCS Scientific Corporation) at 37° C. For experiments with purified hemoglobin, hemoglobin (20 μM as tetramer) was dissolved in DPBS and antifoam (0.2 vol %, Catalog No. AFA-25, TCS Scientific Corporation) was added to prevent foaming of the sample. Compounds were prepared in DMSO stock solutions then added to hemoglobin. The final concentration of DMSO was adjusted to 5 vol %. For experiments with RBCs, blood was drawn from volunteers into EDTA tubes. The blood was centrifuged for 15 min at 3,000 rpm at 4° C. The supernatant was discarded, and RBCs were washed three times with DPBS, pH 7.4 by dilution to 50% hematocrit. The washed RBCs were then diluted with Hemox solution (mixture of N-[Tris(hydroxymethyl)methyl]-2-aminoethanesulfonic acid (TES, 30 mM), sodium chloride (135 mM), and potassium chloride (5 mM) in water (pH 7.4), TCS Scientific Corporation), and mixed with the test compound stock solutions. When known allosteric effectors NEM (400 μM), 5HMF (2 mM) or formamidine disulfide (400 μM) were mixed with RBCs (20 μM as hemoglobin tetramer) for 1 h at 37° C., P₅₀ was reduced to 8 Torr, 9 Torr, and 9 Torr respectively, which correlated with previous reports (see Garel et al., European Journal of Biochemistry, 1982, 123, 513-519). Data was analyzed by two-tailed, unpaired Student's t test using Microsoft Excel 2007.

### Example 13

### Evaluation of Synergistic Reduction of Oxygen Binding Affinity by the Combination of Compound 1 with DPG or IHP

The synergistic reduction of the oxygen binding affinity by Compound 1, DPG, and IHP was evaluated by measuring the ODC in DPBS containing DMSO (5 vol %) at 37° C. using the HEMOX analyzer. A saturating effect on P₅₀ was first determined by adding an increasing concentration of 2,3-DPG and IHP. No increase of P₅₀ of hemoglobin was observed after an addition of a 30-fold molar excess of 2,3-DPG or IHP to hemoglobin. Hemoglobin (20 μM as tetramer) was pre-incubated with either 2,3-DPG (600 μM) or IHP (600 μM) followed by addition of Compound 1 (0, 50, 100, 150 and 200 μM) to the hemoglobin.

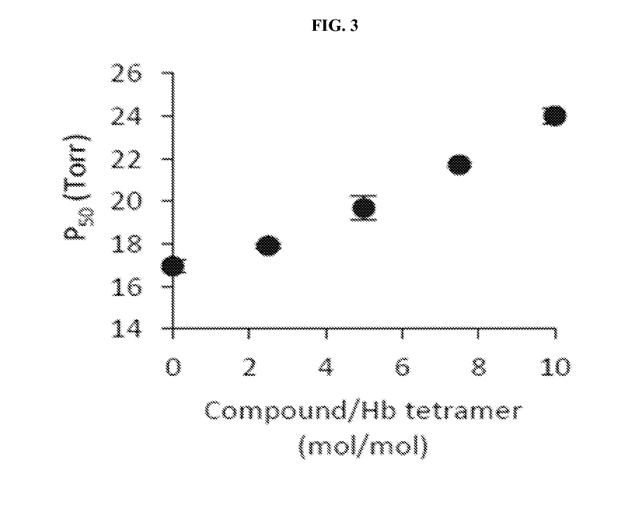

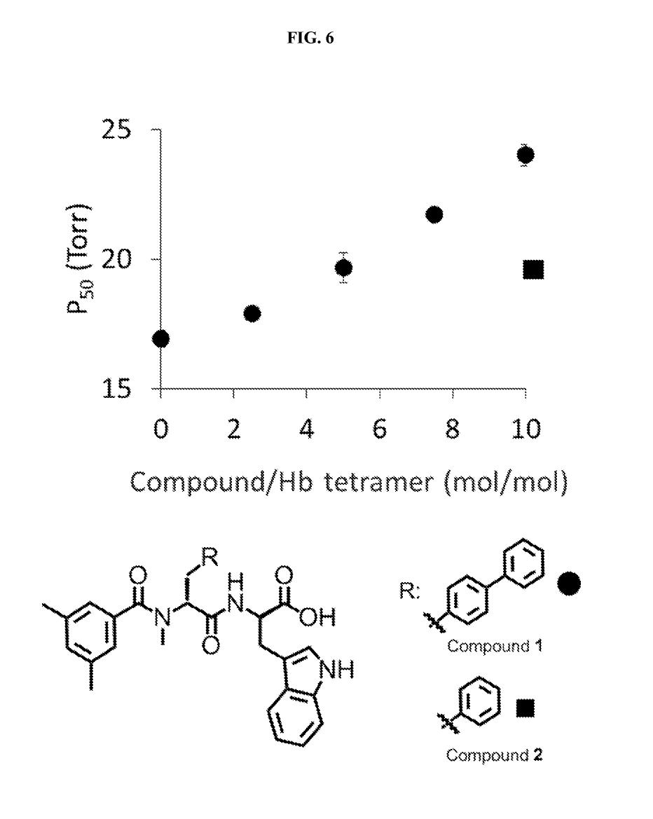

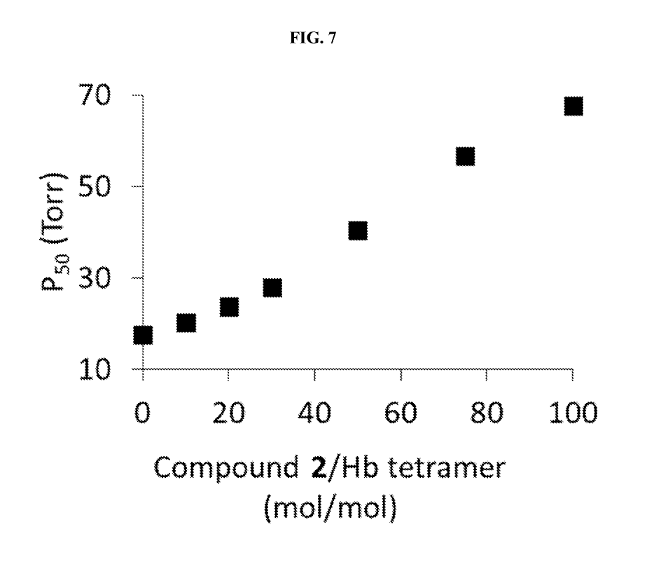

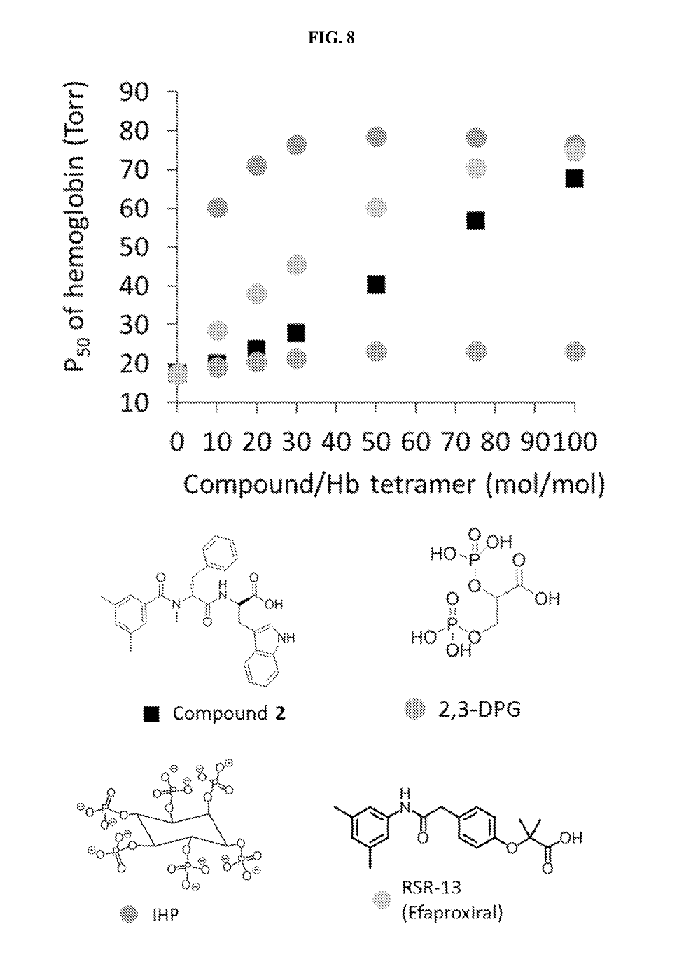

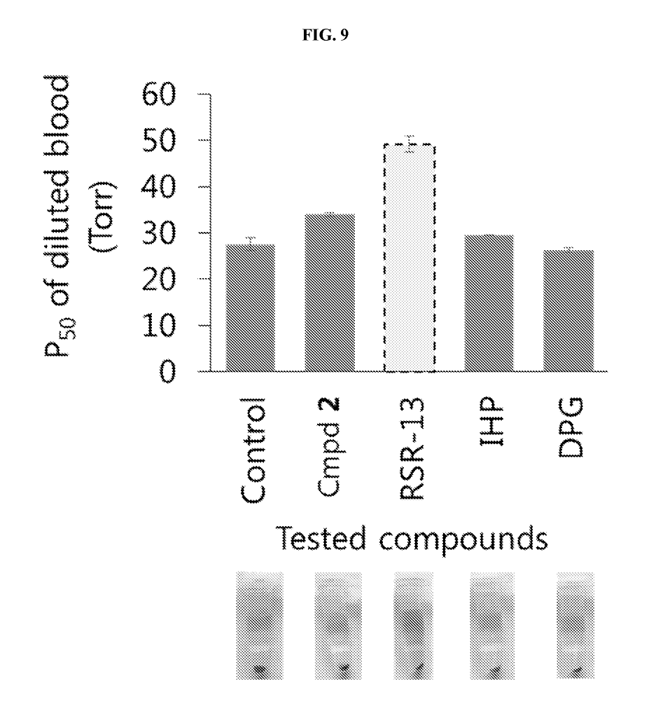

Addition of Compound 1 (200 μM) increased the P₅₀ of hemoglobin (20 μM) from 17±0.3 Torr to 24±0.4 Torr (FIG. 3). This allosteric effect was greater than that of 2,3-DPG (200 μM, P50: 19±0.1 Torr, p<0.001). The increase of P₅₀ induced by Compounds 1 and 2 was also concentration-dependent (FIG. 3 and FIG. 6-7). The effect of dose dependency of Compound 2 on P₅₀ was also compared to three known “right-shifters” (i.e., compounds that enhance oxygen release), in purified human hemoglobin (FIG. 8) and human blood (FIG. 9). These data show that Compound 2 increased the oxygen affinity of red blood cells (RBCs) without hemolysis

Furthermore, a synergistic reduction of oxygen binding affinity when Compound 1 and 2,3-DPG were both added to hemoglobin was observed. Addition of Compound 1 (200 μM) to hemoglobin (20 μM) that was pre-saturated by binding of 2,3-DPG (600 μM) further increased the P50 from 23±0.7 (hemoglobin with 2,3-DPG) to 29±0.5 Torr (hemoglobin with 2,3-DPG and Compound 2, p<0.001). The combination of IHP and Compound 1 did not induce a synergistic reduction of oxygen binding affinity.

Incubation of RBCs with Compound 1 (20 μM) in DPBS with 5% DMSO resulted in hemolysis of RBCs. P₅₀ data for ten test compounds (with purified hemoglobin and whole blood) are shown below in Table 3.

TABLE 3
P₅₀ Data of Compounds 1-10 with free Hb and whole blood

| | P₅₀ with | P₅₀ of |
|---|---|---|
| | purified | whole |
| Compound # | hemoglobin | blood |
| 1 | 24 | Hemolysis |
| 2 | 20 | N/T^{a,b} |
| 3 | 19 | N/T^{b} |
| 4 | 20 | N/T^{b} |
| 5 | 18 | N/T^{b} |
| 6 | 18 | N/T |
| 7 | N/T | N/T |
| 8 | 48 | 32^{c} |
| 9 | 28 | 33^{c} |
| 10 | 23 | 31^{c} |

^{a}N/T = not tested
^{b}P₅₀ of the purified hemoglobin alone and whole blood alone was 17 and 26 Torr respectively..
^{c}does not hemolyze.
The concentration of hemoglobin and tested compounds in the sample was 20 μM and 120 μM respectively.

### Example 14

### Crystallization of Hemoglobin with Compound 1

To obtain unliganded hemoglobin structure in complex with 1, freshly prepared solution of 1 in DMSO was incubated with deoxygenated hemoglobin (deoxyHb) (30-50 mg/mL) for 1 h at a hemoglobin tetramer-1 molar ratio of 1:5, followed by crystallization with a low-salt precipitant (0.2 M sodium acetate trihydrate, 0.1 M sodium cacodylate trihydrate, pH 6.6 and 30% PEG 8000). The crystals were washed in a cryo-protectant solution containing mother liquor and glycerol (3:1 ratio) prior to data collection. The complex crystallized in the orthorhombic space group P2₁2₁2 with typical cell dimensions a=96 Å, b=98 Å and c=65 Å with one functional tetramer per asymmetric unit.

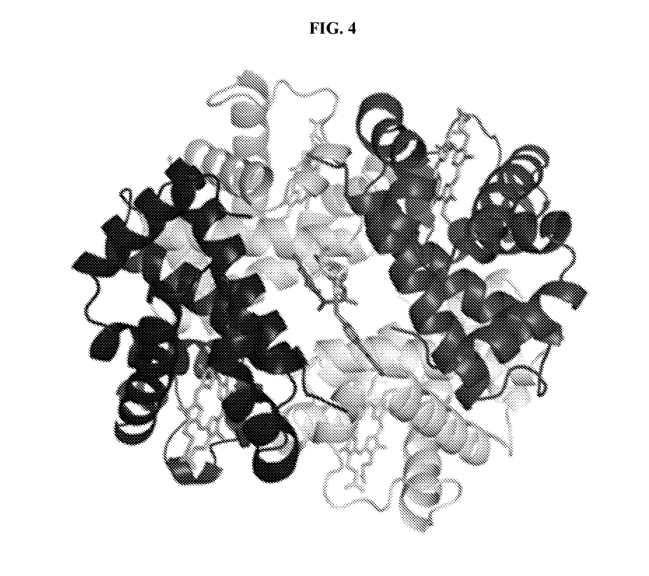

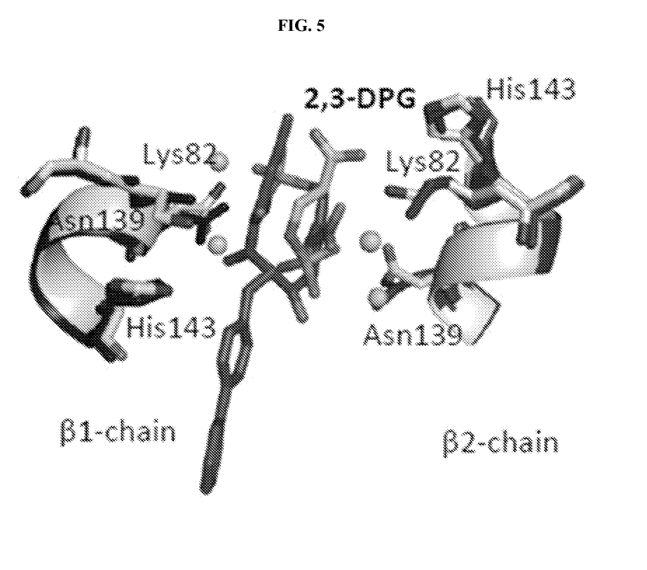

X-ray crystallographic analysis of deoxyHb complexed with Compound 1 revealed that Compound 1 non-covalently binds at the β-cleft on the dyad axis, within the 2,3-DPG binding site (FIG. 4-5). The refined model of the complex contains one hemoglobin tetramer, one 2,3-DPG molecule at the β-cleft in two alternate conformations, and one Compound 1 molecule, also at the β-cleft in one conformation (FIG. 4-5). When compared to the crystal structure of deoxyHb (PDB ID: 2DN2 or 2HHB), the geometry of the β-cleft is not changed by binding of 2,3-DPG and Compound 1 except for slight movement of the side-chains of β-His143, β-Asn139, and β-Lys82 (FIG. 5). The indole carboxyl group of Compound 1 has both water-mediated and direct hydrogen-bond interactions with the amine of β-Lys82, while the indole nitrogen and indole amide nitrogen make direct and water-mediated interactions with the side-chain of β-Asn139, respectively. In effect, Compound 2 and 2,3-DPG are bound to deoxyHb by interactions with β-Lys82, β-Asn139, and β-His143 in a symmetry-related fashion, tying together the two β-subunits and leading to stabilization of the T state.

Diffraction data of all crystals were collected at 100 K with a Rigaku IV ++ image plate detector using a CuKα X-rays (k=1.54 Å) from a MicroMax-007 source fitted with Varimax Confocal optics (Rigaku, The Woodlands, Tex.). The datasets were processed with the d*trek software (Rigaku) and the CCP4 suite of programs.

### Example 15

### Structure Determination of deoxyHb in Complex with Compound 1

Initial phase for the deoxyHb-1 complex structure was obtained by a molecular replacement method with the program CNS, using human deoxyHb α1α1β2β2 tetramer structure (PDB code 2DN2) as a search model. Subsequent structure refinements were performed with the CNS program. Model building and correction were carried out using the graphic program COOT. The initial electron density map of the deoxyHb-1 complex without any ligand and water molecules showed a strong positive difference density on the dyad axis at the β-cleft and 1 was modeled into the density. Subsequent refinement of the structure still resulted in significant presence of positive difference density around the 1 position. It became apparent that the bound entity at the β-cleft is a mixture of 2,3-DPG and 1, which then modeled and were refined at occupancies of 0.66 and 0.37, respectively. The deoxyHb-1 complex was refined to R-factor and R-free of 20.3/22.8 to 1.80 Å.

The atomic coordinates and structure factor files have been deposited in the RCSB Protein Data Bank with accession code 4L7Y for deoxyHb-1. All the protein structures were drawn using PyMOL (Delano Scientific, 2007; http://www.pymol.org).

A number of embodiments of the invention have been described. Nevertheless, it will be understood that various modifications may be made without departing from the spirit and scope of the invention. The references, patents, and patent applications supra are incorporated herein by reference in its entirety.

1. A composition suitable for administration to an individual, comprising red blood cells, treated with a compound of Formula I: 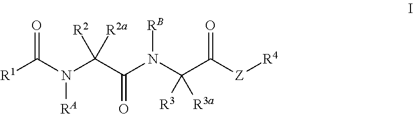
or a pharmaceutically acceptable salt thereof, wherein:
R^{A} is selected from H and C₁₋₃ alkyl;
R^{B} is selected from H and C₁₋₃ alkyl;
R¹ is selected from C₁₋₆ alkyl, C₂₋₆ alkenyl, C₂₋₆ alkynyl, C₁₋₆ haloalkyl, C₃₋₁₀ monocyclic or bicyclic cycloalkyl, 4-10 membered heterocycloalkyl, 6-10 membered aryl, 5-10 membered heteroaryl, C₃₋₁₀ monocyclic or bicyclic cycloalkyl-C₁₋₃ alkylene, 4-10 membered heterocycloalkyl-C₁₋₃ alkylene, 6-10 membered aryl-C₁₋₃ alkylene, and 5-10 membered heteroaryl-C₁₋₃ alkylene, each of which is optionally substituted by 1, 2, 3, or 4 groups independently selected from halo, NO₂, CN, C₁₋₆ alkyl, C₂₋₆ alkenyl, C₂₋₆ alkynyl, C₁₋₆ haloalkyl, Cy¹, —C₁₋₃ alkylene-Cy¹, OR^{a1}, SR^{a1}, C(═O)R^{b1}, C(═O)NR^{c1}R^{d1}, C(═O)OR^{a1}, OC(═O)R^{b1}, OC(═O)NR^{c1}R^{d1}, C(═NR^{e})NR^{c1}R^{d1}, NR^{c1}C(═NR^{e})NR^{c1}R^{d1}, NR^{c1}R^{d1}, NR^{c1}C(═O)R^{b1}, NR^{c1}C(═O)OR^{b1}, NR^{c1}C(═O)NR^{c1}R^{d1}, NR^{c1}S(═O)R^{b}, NR^{c1}S(═O)₂R^{b1}, NR^{c1}S(═O)₂NR^{c1}R^{d1}, S(═O)R^{b1}, S(═O)NR^{c1}R^{d1}, S(═O)₂R^{b1}, and S(═O)₂NR^{c1}R^{d1};
R² is selected from C₁₋₆ alkyl, C₂₋₆ alkenyl, C₂₋₆ alkynyl, C₁₋₆ haloalkyl, Cy, and —C₁₋₃ alkylene-Cy, wherein C₁₋₆ alkyl, C₂₋₆ alkenyl, C₂₋₆ alkynyl, and C₁₋₆ haloalkyl are each optionally substituted by 1, 2, 3, or 4 groups independently selected from CN, OR^{a2}, SR^{a2}, C(═O)R^{b2}, C(═O)NR^{c2}R^{d2}, C(═O)OR^{a2}, OC(═O)R^{b2}, OC(═O)NR^{c2}R^{d2}, C(═NR^{e})NR^{c2}R^{d2}, NR^{c2}C(═NR^{e})NR^{c2}R^{d2}, NR^{c2}R^{d2}, NR^{c2}C(═O)R^{b2}, NR^{c2}C(═O)OR^{b2}, NR^{c2}C(═O)NR^{c2}R^{d2}, NR^{c2}S(═O)R^{b}, NR^{c2}S(═O)₂R^{b2}, NR^{c2}S(═O)₂NR^{c2}R^{d2}, S(═O)R^{b2}, S(═O)NR^{c2}R^{d2}, S(═O)₂R^{b2}, and S(═O)₂NR^{c2}R^{d2};
R^{2a} is selected from H and C₁₋₃ alkyl;
R³ is selected from H, C₁₋₆ alkyl, C₂₋₆ alkenyl, C₂₋₆ alkynyl, C₁₋₆ haloalkyl, C₃₋₁₀ monocyclic or bicyclic cycloalkyl, 4-10 membered heterocycloalkyl, 6-10 membered aryl, 5-10 membered heteroaryl, C₃₋₁₀ monocyclic or bicyclic cycloalkyl-C₁₋₃ alkylene, 4-10 membered heterocycloalkyl-C₁₋₃ alkylene, 6-10 membered aryl-C₁₋₃ alkylene, and 5-10 membered heteroaryl-C₁₋₃ alkylene, wherein said C₁₋₆ alkyl, C₂₋₆ alkenyl, C₂₋₆ alkynyl, C₁₋₆ haloalkyl, C₃₋₁₀ monocyclic or bicyclic cycloalkyl, 4-10 membered heterocycloalkyl, 6-10 membered aryl, 5-10 membered heteroaryl, C₃₋₁₀ monocyclic or bicyclic cycloalkyl-C₁₋₃ alkylene, 4-10 membered heterocycloalkyl-C₁₋₃ alkylene, 6-10 membered aryl-C₁₋₃ alkylene, and 5-10 membered heteroaryl-C₁₋₃ alkylene are each optionally substituted by 1, 2, 3, or 4 independently selected R^{x} groups;
R^{3a} is selected from H and C₁₋₃ alkyl;
Z is selected from O or NR^{C};
R^{C} is H or C₁₋₃ alkyl;
R⁴ is H or C₁₋₃ alkyl;
each Cy is independently selected from C₃₋₁₀ monocyclic or bicyclic cycloalkyl, 4-10 membered heterocycloalkyl, 6-10 membered aryl, and 5-10 membered heteroaryl, each of which is optionally substituted by 1, 2, 3, or 4 substituents independently selected from C₁₋₆ alkyl, C₂₋₆ alkenyl, C₂₋₆ alkynyl, C₁₋₆ haloalkyl, Cy², —C₁₋₃ alkylene-Cy², halo, NO₂, CN, OR^{a2}, SR^{a2}, C(═O)R^{b2}, C(═O)NR^{c2}R^{d2}, C(═O)OR^{a2}, OC(═O)R^{b2}, OC(═O)NR^{c2}R^{d2}, C(═NR^{e})NR^{c2}R^{d2}, NR^{c2}C(═NR^{e})NR^{c2}R^{d2}, NR^{c2}R^{d2}, NR^{c2}C(═O)R^{b2}, NR^{c2}C(═O)OR^{b2}, NR^{c2}C(═O)NR^{c2}R^{d2}, NR^{c2}S(═O)R^{b}, NR^{c2}S(═O)₂R^{b2}, NR^{c2}S(═O)₂NR^{c2}R^{d2}, S(═O)R^{b2}, S(═O)NR^{c2}R^{d2}, S(═O)₂R^{b2}, and S(═O)₂NR^{c2}R^{d2};
each R^{a1}, R^{c1}, and R^{d1} are independently selected from H, C₁₋₆ alkyl, C₂₋₆ alkenyl, C₂₋₆ alkynyl, C₁₋₆ haloalkyl, C₃₋₇ monocyclic cycloalkyl, 4-6 membered heterocycloalkyl, phenyl, 5-6 membered heteroaryl, C₃₋₇ monocyclic cycloalkyl-C₁₋₃ alkylene, 4-6 membered heterocycloalkyl-C₁₋₃ alkylene, phenyl-C₁₋₃ alkylene, and 5-6 membered heteroaryl-C₁₋₃ alkylene; wherein said C₁₋₆ alkyl, C₂₋₆ alkenyl, C₂₋₆ alkynyl, C₁₋₆ haloalkyl, C₃₋₇ monocyclic cycloalkyl, 4-6 membered heterocycloalkyl, phenyl, 5-6 membered heteroaryl, C₃₋₇ monocyclic cycloalkyl-C₁₋₃ alkylene, 4-6 membered heterocycloalkyl-C₁₋₃ alkylene, phenyl-C₁₋₃ alkylene, and 5-6 membered heteroaryl-C₁₋₃ alkylene are each optionally substituted with 1, 2, or 3 independently selected R^{x} groups;
each R^{b1} is independently selected from C₁₋₆ alkyl, C₂₋₆ alkenyl, C₂₋₆ alkynyl, C₁₋₆ haloalkyl, C₃₋₇ monocyclic cycloalkyl, 4-6 membered heterocycloalkyl, phenyl, 5-6 membered heteroaryl, C₃₋₇ monocyclic cycloalkyl-C₁₋₃ alkylene, 4-6 membered heterocycloalkyl-C₁₋₃ alkylene, phenyl-C₁₋₃ alkylene, and 5-6 membered heteroaryl-C₁₋₃ alkylene; each of which are each optionally substituted with 1, 2, or 3 independently selected R^{x} groups;
each R^{a2}, R^{c2}, and R^{d2} are independently selected from H, C₁₋₆ alkyl, C₂₋₆ alkenyl, C₂₋₆ alkynyl, C₁₋₆ haloalkyl, C₃₋₇ monocyclic cycloalkyl, 4-6 membered heterocycloalkyl, phenyl, 5-6 membered heteroaryl, C₃₋₇ monocyclic cycloalkyl-C₁₋₃ alkylene, 4-6 membered heterocycloalkyl-C₁₋₃ alkylene, phenyl-C₁₋₃ alkylene, and 5-6 membered heteroaryl-C₁₋₃ alkylene; wherein said C₁₋₆ alkyl, C₂₋₆ alkenyl, C₂₋₆ alkynyl, C₁₋₆ haloalkyl, C₃₋₇ monocyclic cycloalkyl, 4-6 membered heterocycloalkyl, phenyl, 5-6 membered heteroaryl, C₃₋₇ monocyclic cycloalkyl-C₁₋₃ alkylene, 4-6 membered heterocycloalkyl-C₁₋₃ alkylene, phenyl-C₁₋₃ alkylene, and 5-6 membered heteroaryl-C₁₋₃ alkylene are each optionally substituted with 1, 2, or 3 independently selected R^{x} groups;
each R^{b2} is independently selected from C₁₋₆ alkyl, C₂₋₆ alkenyl, C₂₋₆ alkynyl, C₁₋₆ haloalkyl, C₃₋₇ monocyclic cycloalkyl, 4-6 membered heterocycloalkyl, phenyl, 5-6 membered heteroaryl, C₃₋₇ monocyclic cycloalkyl-C₁₋₃ alkylene, 4-6 membered heterocycloalkyl-C₁₋₃ alkylene, phenyl-C₁₋₃ alkylene, and 5-6 membered heteroaryl-C₁₋₃ alkylene; each of which are each optionally substituted with 1, 2, or 3 independently selected R^{x} groups;
each R^{e} is independently selected from H, CN, OH, C₁₋₄ alkyl, C₁₋₄ alkoxy, NO₂, C(O)(C₁₋₄ alkyl), and S(═O)₂(C₁₋₄ alkyl);
each Cy¹ is independently selected from C₃₋₇ monocyclic cycloalkyl, 4-6 membered heterocycloalkyl, phenyl, and 5-6 membered heteroaryl, each of which is optionally substituted by 1, 2, 3, or 4 independently selected R^{x} groups;
each Cy² is independently selected from C₃₋₁₀ monocyclic or bicyclic cycloalkyl, 4-10 membered heterocycloalkyl, 6-10 membered aryl, 5-10 membered heteroaryl, each of which is optionally substituted by 1, 2, 3, or 4 groups independently selected from C₁₋₆ alkyl, C₂₋₆ alkenyl, C₂₋₆ alkynyl, C₁₋₆ haloalkyl, halo, NO₂, CN, OR^{a2}, SR^{a2}, C(═O)R^{b2}, C(═O)NR^{c2}R^{d2}, C(═O)OR^{a2}, OC(═O)R^{b2}, OC(═O)NR^{c2}R^{d2}, C(═NR^{e})NR^{c2}R^{d2}, NR^{c2}C(═NR^{e})NR^{c2}R^{d2}, NR^{c2}R^{d2}, NR^{c2}C(═O)R^{b2}, NR^{c2}C(═O)OR^{b2}, NR^{c2}C(═O)NR^{c2}R^{d2}, NR^{c2}S(═O)R^{b}, NR^{c2}S(═O)₂R^{b2}, NR^{c2}S(═O)₂NR^{c2}R^{d2}, S(═O)R^{b2}, S(═O)NR^{c2}R^{d2}, S(═O)₂R^{b2}, and S(═O)₂NR^{c2}R^{d2}; and
each R^{x} is independently selected from halo, OH, NO₂, CN, C₁₋₆ alkyl, C₂₋₆ alkenyl, C₂₋₆ alkynyl, C₁₋₆ haloalkyl, cyano-C₁₋₆ alkyl, HO—C₁₋₆ alkyl, C₁₋₆ alkoxy-C₁₋₆ alkyl, C₁₋₆ alkoxy, C₁₋₆ haloalkoxy, amino, C₁₋₆ alkylamino, di(C₁₋₆ alkyl)amino, thio, C₁₋₆ alkylthio, C₁₋₆ alkylsulfinyl, C₁₋₆ alkylsulfonyl, carbamyl, C₁₋₆ alkylcarbamyl, di(C₁₋₆ alkyl)carbamyl, carboxy, C₁₋₆ alkylcarbonyl, C₁₋₆ alkoxycarbonyl, C₁₋₆ alkylcarbonyloxy, C₁₋₆ alkylcarbonylamino, C₁₋₆ alkylsulfonylamino, aminosulfonyl, C₁₋₆ alkylaminosulfonyl, di(C₁₋₆ alkyl)aminosulfonyl, aminosulfonylamino, C₁₋₆ alkylaminosulfonylamino, di(C₁₋₆ alkyl)aminosulfonylamino, aminocarbonylamino, C₁₋₆ alkylaminocarbonylamino, and di(C₁₋₆ alkyl)aminocarbonylamino.

2. A method of treating acute or chronic ischemia, anemia, coronary infarction, chronic pulmonary disease, congestive heart failure, diabetes, diabetic neuropathy, myocardial infarction, stroke, peripheral vascular disease, peripheral vascular insufficiency, intermittent claudication, circulatory shock, hemorrhagic shock, chronic hypoxia, altitude sickness, arteriosclerosis, respiratory alkalemia, metabolic alkalosis, reduced lung capacity, gangrene, anaerobic infections, carbon monoxide poisoning, nitric oxide poisoning, or cyanide poisoning in an individual in need thereof, comprising administering to said individual a composition of claim 1 or a therapeutically effective amount of a compound of Formula I: 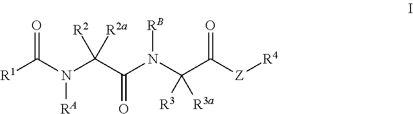
or a pharmaceutically acceptable salt thereof, wherein:
R^{A} is selected from H and C₁₋₃ alkyl;
R^{B} is selected from H and C₁₋₃ alkyl;
R¹ is selected from C₁₋₆ alkyl, C₂₋₆ alkenyl, C₂₋₆ alkynyl, C₁₋₆ haloalkyl, C₃₋₁₀ monocyclic or bicyclic cycloalkyl, 4-10 membered heterocycloalkyl, 6-10 membered aryl, 5-10 membered heteroaryl, C₃₋₁₀ monocyclic or bicyclic cycloalkyl-C₁₋₃ alkylene, 4-10 membered heterocycloalkyl-C₁₋₃ alkylene, 6-10 membered aryl-C₁₋₃ alkylene, and 5-10 membered heteroaryl-C₁₋₃ alkylene, each of which is optionally substituted by 1, 2, 3, or 4 groups independently selected from halo, NO₂, CN, C₁₋₆ alkyl, C₂₋₆ alkenyl, C₂₋₆ alkynyl, C₁₋₆ haloalkyl, Cy¹, —C₁₋₃ alkylene-Cy¹, OR^{a1}, SR^{a1}, C(═O)R^{b1}, C(═O)NR^{c1}R^{d1}, C(═O)OR^{a1}, OC(═O)R^{b1}, OC(═O)NR^{c1}R^{d1}, C(═NR^{e})NR^{c1}R^{d1}, NR^{c1}C(═NR^{e})NR^{c1}R^{d1} NR^{c1}R^{d1}, NR^{c1}C(═O)R^{b1}, NR^{c1}C(═O)OR^{b1}, NR^{c1}C(═O)NR^{c1}R^{d1}, NR^{c1}S(═O)R^{b}, NR^{c1} S(═O)₂R^{b1}, NR^{c1}S(═O)₂NR^{c1}R^{d1}, S(═O)R^{b1}, S(═O)NR^{c1}R^{d1}, S(═O)₂R^{b1}, and S(═O)₂NR^{c1}R^{d1};
R² is selected from C₁₋₆ alkyl, C₂₋₆ alkenyl, C₂₋₆ alkynyl, C₁₋₆ haloalkyl, Cy, and —C₁₋₃ alkylene-Cy, wherein C₁₋₆ alkyl, C₂₋₆ alkenyl, C₂₋₆ alkynyl, and C₁₋₆ haloalkyl are each optionally substituted by 1, 2, 3, or 4 groups independently selected from CN, OR^{a2}, SR^{a2}, C(═O)R^{b2}, C(═O)NR^{c2}R^{d2}, C(═O)OR^{a2}, OC(═O)R^{b2}, OC(═O)NR^{c2}R^{d2}, C(═NR^{e})NR^{c2}R^{d2}, NR^{c2}C(═NR^{e})NR^{c2}R^{d2}, NR^{c2}R^{d2}, NR^{c2}C(═O)R^{b2}, NR^{c2}C(═O)OR^{b2}, NR^{c2}C(═O)NR^{c2}R^{d2}, NR^{c2}S(═O)R^{b}, NR^{c2}S(═O)₂R^{b2}, NR^{c2}S(═O)₂NR^{c2}R^{d2}, S(═O)R^{b2}, S(═O)NR^{c2}R^{d2}, S(═O)₂R^{b2}, and S(═O)₂NR^{c2}R^{d2};
R^{2a} is selected from H and C₁₋₃ alkyl;
R³ is selected from H, C₁₋₆ alkyl, C₂₋₆ alkenyl, C₂₋₆ alkynyl, C₁₋₆ haloalkyl, C₃₋₁₀ monocyclic or bicyclic cycloalkyl, 4-10 membered heterocycloalkyl, 6-10 membered aryl, 5-10 membered heteroaryl, C₃₋₁₀ monocyclic or bicyclic cycloalkyl-C₁₋₃ alkylene, 4-10 membered heterocycloalkyl-C₁₋₃ alkylene, 6-10 membered aryl-C₁₋₃ alkylene, and 5-10 membered heteroaryl-C₁₋₃ alkylene, wherein said C₁₋₆ alkyl, C₂₋₆ alkenyl, C₂₋₆ alkynyl, C₁₋₆ haloalkyl, C₃₋₁₀ monocyclic or bicyclic cycloalkyl, 4-10 membered heterocycloalkyl, 6-10 membered aryl, 5-10 membered heteroaryl, C₃₋₁₀ monocyclic or bicyclic cycloalkyl-C₁₋₃ alkylene, 4-10 membered heterocycloalkyl-C₁₋₃ alkylene, 6-10 membered aryl-C₁₋₃ alkylene, and 5-10 membered heteroaryl-C₁₋₃ alkylene are each optionally substituted by 1, 2, 3, or 4 independently selected R^{x} groups;
R^{3a} is selected from H and C₁₋₃ alkyl;
Z is selected from O or NR^{C};
R^{C} is H or C₁₋₃ alkyl;
R⁴ is H or C₁₋₃ alkyl;
each Cy is independently selected from C₃₋₁₀ monocyclic or bicyclic cycloalkyl, 4-10 membered heterocycloalkyl, 6-10 membered aryl, and 5-10 membered heteroaryl, each of which is optionally substituted by 1, 2, 3, or 4 substituents independently selected from C₁₋₆ alkyl, C₂₋₆ alkenyl, C₂₋₆ alkynyl, C₁₋₆ haloalkyl, Cy², —C₁₋₃ alkylene-Cy², halo, NO₂, CN, OR^{a2}, SR^{a2}, C(═O)R^{b2}, C(═O)NR^{c2}R^{d2}, C(═O)OR^{a2}, OC(═O)R^{b2}, OC(═O)NR^{c2}R^{d2}, C(═NR^{e})NR^{c2}R^{d2}, NR^{c2}C(═NR^{e})NR^{c2}R^{d2}, NR^{c2}R^{d2}, NR^{c2}C(═O)R^{b2}, NR^{c2}C(═O)OR^{b2}, NR^{c2}C(═O)NR^{c2}R^{d2}, NR^{c2}S(═O)R^{b}, NR^{c2}S(═O)₂R^{b2}, NR^{c2}S(═O)₂NR^{c2}R^{d2}, S(═O)R^{b2}, S(═O)NR^{c2}, R^{d2}, S(═O)₂R^{b2}, and S(═O)₂NR^{c2}R^{d2};
each R^{a1}, R^{c1}, and R^{d1} are independently selected from H, C₁₋₆ alkyl, C₂₋₆ alkenyl, C₂₋₆ alkynyl, C₁₋₆ haloalkyl, C₃₋₇ monocyclic cycloalkyl, 4-6 membered heterocycloalkyl, phenyl, 5-6 membered heteroaryl, C₃₋₇ monocyclic cycloalkyl-C₁₋₃ alkylene, 4-6 membered heterocycloalkyl-C₁₋₃ alkylene, phenyl-C₁₋₃ alkylene, and 5-6 membered heteroaryl-C₁₋₃ alkylene; wherein said C₁₋₆ alkyl, C₂₋₆ alkenyl, C₂₋₆ alkynyl, C₁₋₆ haloalkyl, C₃₋₇ monocyclic cycloalkyl, 4-6 membered heterocycloalkyl, phenyl, 5-6 membered heteroaryl, C₃₋₇ monocyclic cycloalkyl-C₁₋₃ alkylene, 4-6 membered heterocycloalkyl-C₁₋₃ alkylene, phenyl-C₁₋₃ alkylene, and 5-6 membered heteroaryl-C₁₋₃ alkylene are each optionally substituted with 1, 2, or 3 independently selected R^{x} groups;
each R^{b1} is independently selected from C₁₋₆ alkyl, C₂₋₆ alkenyl, C₂₋₆ alkynyl, C₁₋₆ haloalkyl, C₃₋₇ monocyclic cycloalkyl, 4-6 membered heterocycloalkyl, phenyl, 5-6 membered heteroaryl, C₃₋₇ monocyclic cycloalkyl-C₁₋₃ alkylene, 4-6 membered heterocycloalkyl-C₁₋₃ alkylene, phenyl-C₁₋₃ alkylene, and 5-6 membered heteroaryl-C₁₋₃ alkylene; each of which are each optionally substituted with 1, 2, or 3 independently selected R^{x} groups;
each R^{a2}, R^{c2}, and R^{d2} are independently selected from H, C₁₋₆ alkyl, C₂₋₆ alkenyl, C₂₋₆ alkynyl, C₁₋₆ haloalkyl, C₃₋₇ monocyclic cycloalkyl, 4-6 membered heterocycloalkyl, phenyl, 5-6 membered heteroaryl, C₃₋₇ monocyclic cycloalkyl-C₁₋₃ alkylene, 4-6 membered heterocycloalkyl-C₁₋₃ alkylene, phenyl-C₁₋₃ alkylene, and 5-6 membered heteroaryl-C₁₋₃ alkylene; wherein said C₁₋₆ alkyl, C₂₋₆ alkenyl, C₂₋₆ alkynyl, C₁₋₆ haloalkyl, C₃₋₇ monocyclic cycloalkyl, 4-6 membered heterocycloalkyl, phenyl, 5-6 membered heteroaryl, C₃₋₇ monocyclic cycloalkyl-C₁₋₃ alkylene, 4-6 membered heterocycloalkyl-C₁₋₃ alkylene, phenyl-C₁₋₃ alkylene, and 5-6 membered heteroaryl-C₁₋₃ alkylene are each optionally substituted with 1, 2, or 3 independently selected R^{x} groups;
each R^{b2} is independently selected from C₁₋₆ alkyl, C₂₋₆ alkenyl, C₂₋₆ alkynyl, C₁₋₆ haloalkyl, C₃₋₇ monocyclic cycloalkyl, 4-6 membered heterocycloalkyl, phenyl, 5-6 membered heteroaryl, C₃₋₇ monocyclic cycloalkyl-C₁₋₃ alkylene, 4-6 membered heterocycloalkyl-C₁₋₃ alkylene, phenyl-C₁₋₃ alkylene, and 5-6 membered heteroaryl-C₁₋₃ alkylene; each of which are each optionally substituted with 1, 2, or 3 independently selected R^{x} groups;
each R^{e} is independently selected from H, CN, OH, C₁₋₄ alkyl, C₁₋₄ alkoxy, NO₂, C(O)(C₁₋₄ alkyl), and S(═O)₂(C₁₋₄ alkyl);
each Cy¹ is independently selected from C₃₋₇ monocyclic cycloalkyl, 4-6 membered heterocycloalkyl, phenyl, and 5-6 membered heteroaryl, each of which is optionally substituted by 1, 2, 3, or 4 independently selected R^{x} groups;
each Cy² is independently selected from C₃₋₁₀ monocyclic or bicyclic cycloalkyl, 4-10 membered heterocycloalkyl, 6-10 membered aryl, 5-10 membered heteroaryl, each of which is optionally substituted by 1, 2, 3, or 4 groups independently selected from C₁₋₆ alkyl, C₂₋₆ alkenyl, C₂₋₆ alkynyl, C₁₋₆ haloalkyl, halo, NO₂, CN, OR^{a2}, SR^{a2}, C(═O)R^{b2}, C(═O)NR^{c2}R^{d2}, C(═O)OR^{a2}, OC(═O)R^{b2}, OC(═O)NR^{c2}R^{d2}, C(═NR^{e})NR^{c2}R^{d2}, NR^{c2}C(═NR^{e})NR^{c2}R^{d2}, NR^{c2}R^{d2}, NR^{c2}C(═O)R^{b2}, NR^{c2}C(═O)OR^{b2}, NR^{c2}C(═O)NR^{c2}R^{d2}, NR^{c2}S(═O)R^{b}, NR^{c2}S(═O)₂R^{b2}, NR^{c2}S(═O)₂NR^{c2}R^{d2}, S(═O)R^{b2}, S(═O)NR^{c2}R^{d2}, S(═O)₂R^{b2}, and S(═O)₂NR^{c2}R^{d2}; and
each R^{x} is independently selected from halo, OH, NO₂, CN, C₁₋₆ alkyl, C₂₋₆ alkenyl, C₂₋₆ alkynyl, C₁₋₆ haloalkyl, cyano-C₁₋₆ alkyl, HO—C₁₋₆ alkyl, C₁₋₆ alkoxy-C₁₋₆ alkyl, C₁₋₆ alkoxy, C₁₋₆ haloalkoxy, amino, C₁₋₆ alkylamino, di(C₁₋₆ alkyl)amino, thio, C₁₋₆ alkylthio, C₁₋₆ alkylsulfinyl, C₁₋₆ alkylsulfonyl, carbamyl, C₁₋₆ alkylcarbamyl, di(C₁₋₆ alkyl)carbamyl, carboxy, C₁₋₆ alkylcarbonyl, C₁₋₆ alkoxycarbonyl, C₁₋₆ alkylcarbonyloxy, C₁₋₆ alkylcarbonylamino, C₁₋₆ alkylsulfonylamino, aminosulfonyl, C₁₋₆ alkylaminosulfonyl, di(C₁₋₆ alkyl)aminosulfonyl, aminosulfonylamino, C₁₋₆ alkylaminosulfonylamino, di(C₁₋₆ alkyl)aminosulfonylamino, aminocarbonylamino, C₁₋₆ alkylaminocarbonylamino, and di(C₁₋₆ alkyl)aminocarbonylamino.

3. The method of claim 1, wherein the ischemia is associated with peripheral vascular disease, coronary occlusion, cerebral vascular accidents, or tissue transplant.

4. A method of treating a cancer in an individual in need thereof, comprising administering to said individual a composition of claim 1 or a therapeutically effective amount of compound of Formula I: 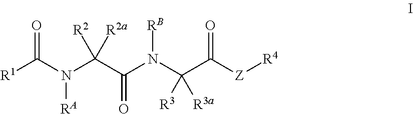
or a pharmaceutically acceptable salt thereof, in combination with radiation therapy; wherein:
R^{A} is selected from H and C₁₋₃ alkyl;
R^{B} is selected from H and C₁₋₃ alkyl;
R¹ is selected from C₁₋₆ alkyl, C₂₋₆ alkenyl, C₂₋₆ alkynyl, C₁₋₆ haloalkyl, C₃₋₁₀ monocyclic or bicyclic cycloalkyl, 4-10 membered heterocycloalkyl, 6-10 membered aryl, 5-10 membered heteroaryl, C₃₋₁₀ monocyclic or bicyclic cycloalkyl-C₁₋₃ alkylene, 4-10 membered heterocycloalkyl-C₁₋₃ alkylene, 6-10 membered aryl-C₁₋₃ alkylene, and 5-10 membered heteroaryl-C₁₋₃ alkylene, each of which is optionally substituted by 1, 2, 3, or 4 groups independently selected from halo, NO₂, CN, C₁₋₆ alkyl, C₂₋₆ alkenyl, C₂₋₆ alkynyl, C₁₋₆ haloalkyl, Cy¹, —C₁₋₃ alkylene-Cy¹, OR^{a1}, SR^{a1}, C(═O)R^{b1}, C(═O)NR^{c1}R^{d1}, C(═O)OR^{a1}, OC(═O)R^{b1}, OC(═O)NR^{c1}R^{d1}, C(═NR^{e})NR^{c1}R^{d1}, NR^{c1}C(═NR^{e})NR^{c1}R^{d1}, NR^{c1}R^{d1}, NR^{c1}C(═O)R^{b1}, NR^{c1}C(═O)OR^{b1}, NR^{c1}C(═O)NR^{c1}R^{d1}, NR^{c1}S(═O)R^{b}, NR^{c1}S(═O)₂R^{b1}, NR^{c1}S(═O)₂NR^{c1}R^{d1}, S(═O)R^{b1}, S(═O)NR^{c1}R^{d1}, S(═O)₂R^{b1}, and S(═O)₂NR^{c1}R^{d1};
R² is selected from C₁₋₆ alkyl, C₂₋₆ alkenyl, C₂₋₆ alkynyl, C₁₋₆ haloalkyl, Cy, and —C₁₋₃ alkylene-Cy, wherein C₁₋₆ alkyl, C₂₋₆ alkenyl, C₂₋₆ alkynyl, and C₁₋₆ haloalkyl are each optionally substituted by 1, 2, 3, or 4 groups independently selected from CN, OR^{a2}, SR^{a2}, C(═O)R^{b2}, C(═O)NR^{c2}R^{d2}, C(═O)OR^{a2}, OC(═O)R^{b2}, OC(═O)NR^{c2}R^{d2}, (═NR^{e})NR^{c2}R^{d2}, NR^{c2}C(═NR^{e})NR^{c2}R^{d2}, NR^{c2}R^{d2}, NR^{c2}C(═O)R^{b2}, NR^{c2}C(═O)OR^{b2}, NR^{c2}C(═O)NR^{c2}R^{d2}, NR^{c2}S(═O)R^{b}, NR^{c2}S(═O)₂R^{b2}, NR^{c2}S(═O)₂NR^{c2}R^{d2}, S(═O)R^{b2}, S(═O)NR^{c2}R^{d2}, S(═O)₂R^{b2}, and S(═O)₂NR^{c2}R^{d2};
R^{2a} is selected from H and C₁₋₃ alkyl;
R³ is selected from H, C₁₋₆ alkyl, C₂₋₆ alkenyl, C₂₋₆ alkynyl, C₁₋₆ haloalkyl, C₃₋₁₀ monocyclic or bicyclic cycloalkyl, 4-10 membered heterocycloalkyl, 6-10 membered aryl, 5-10 membered heteroaryl, C₃₋₁₀ monocyclic or bicyclic cycloalkyl-C₁₋₃ alkylene, 4-10 membered heterocycloalkyl-C₁₋₃ alkylene, 6-10 membered aryl-C₁₋₃ alkylene, and 5-10 membered heteroaryl-C₁₋₃ alkylene, wherein said C₁₋₆ alkyl, C₂₋₆ alkenyl, C₂₋₆ alkynyl, C₁₋₆ haloalkyl, C₃₋₁₀ monocyclic or bicyclic cycloalkyl, 4-10 membered heterocycloalkyl, 6-10 membered aryl, 5-10 membered heteroaryl, C₃₋₁₀ monocyclic or bicyclic cycloalkyl-C₁₋₃ alkylene, 4-10 membered heterocycloalkyl-C₁₋₃ alkylene, 6-10 membered aryl-C₁₋₃ alkylene, and 5-10 membered heteroaryl-C₁₋₃ alkylene are each optionally substituted by 1, 2, 3, or 4 independently selected R^{x} groups;
R^{3a} is selected from H and C₁₋₃ alkyl;
Z is selected from O or NR^{C};
R^{C} is H or C₁₋₃ alkyl;
R⁴ is H or C₁₋₃ alkyl;
each Cy is independently selected from C₃₋₁₀ monocyclic or bicyclic cycloalkyl, 4-10 membered heterocycloalkyl, 6-10 membered aryl, and 5-10 membered heteroaryl, each of which is optionally substituted by 1, 2, 3, or 4 substituents independently selected from C₁₋₆ alkyl, C₂₋₆ alkenyl, C₂₋₆ alkynyl, C₁₋₆ haloalkyl, Cy², —C₁₋₃ alkylene-Cy², halo, NO₂, CN, OR^{a2}, SR^{a2}, C(═O)R^{b2}, C(═O)NR^{c2}R^{d2}, C(═O)OR^{a2}, OC(═O)R^{b2}, OC(═O)NR^{c2}R^{d2}; C(═NR^{e})NR^{c2}R^{d2}, NR^{c2}C(═NR^{e})NR^{c2}R^{d2}, NR^{c2}R^{d2}, NR^{c2}C(═O)R^{b2}, NR^{c2}C(═O)OR^{b2}, NR^{c2}C(═O)NR^{c2}R^{d2}, NR^{c2}S(═O)R^{b}, NR^{c2}S(═O)₂R^{b2}, NR^{c2}S(═O)₂NR^{c2}R^{d2}, S(═O)R^{b2}, S(═O)NR^{c2}R^{d2}, S(═O)₂R^{b2}, and S(═O)₂NR^{c2}R^{d2};
each R^{a1}, R^{c1}, and R^{d1} are independently selected from H, C₁₋₆ alkyl, C₂₋₆ alkenyl, C₂₋₆ alkynyl, C₁₋₆ haloalkyl, C₃₋₇ monocyclic cycloalkyl, 4-6 membered heterocycloalkyl, phenyl, 5-6 membered heteroaryl, C₃₋₇ monocyclic cycloalkyl-C₁₋₃ alkylene, 4-6 membered heterocycloalkyl-C₁₋₃ alkylene, phenyl-C₁₋₃ alkylene, and 5-6 membered heteroaryl-C₁₋₃ alkylene; wherein said C₁₋₆ alkyl, C₂₋₆ alkenyl, C₂₋₆ alkynyl, C₁₋₆ haloalkyl, C₃₋₇ monocyclic cycloalkyl, 4-6 membered heterocycloalkyl, phenyl, 5-6 membered heteroaryl, C₃₋₇ monocyclic cycloalkyl-C₁₋₃ alkylene, 4-6 membered heterocycloalkyl-C₁₋₃ alkylene, phenyl-C₁₋₃ alkylene, and 5-6 membered heteroaryl-C₁₋₃ alkylene are each optionally substituted with 1, 2, or 3 independently selected R^{x} groups;
each R^{b1} is independently selected from C₁₋₆ alkyl, C₂₋₆ alkenyl, C₂₋₆ alkynyl, C₁₋₆ haloalkyl, C₃₋₇ monocyclic cycloalkyl, 4-6 membered heterocycloalkyl, phenyl, 5-6 membered heteroaryl, C₃₋₇ monocyclic cycloalkyl-C₁₋₃ alkylene, 4-6 membered heterocycloalkyl-C₁₋₃ alkylene, phenyl-C₁₋₃ alkylene, and 5-6 membered heteroaryl-C₁₋₃ alkylene; each of which are each optionally substituted with 1, 2, or 3 independently selected R^{x} groups;
each R^{a2}, R^{c2}, and R^{d2} are independently selected from H, C₁₋₆ alkyl, C₂₋₆ alkenyl, C₂₋₆ alkynyl, C₁₋₆ haloalkyl, C₃₋₇ monocyclic cycloalkyl, 4-6 membered heterocycloalkyl, phenyl, 5-6 membered heteroaryl, C₃₋₇ monocyclic cycloalkyl-C₁₋₃ alkylene, 4-6 membered heterocycloalkyl-C₁₋₃ alkylene, phenyl-C₁₋₃ alkylene, and 5-6 membered heteroaryl-C₁₋₃ alkylene; wherein said C₁₋₆ alkyl, C₂₋₆ alkenyl, C₂₋₆ alkynyl, C₁₋₆ haloalkyl, C₃₋₇ monocyclic cycloalkyl, 4-6 membered heterocycloalkyl, phenyl, 5-6 membered heteroaryl, C₃₋₇ monocyclic cycloalkyl-C₁₋₃ alkylene, 4-6 membered heterocycloalkyl-C₁₋₃ alkylene, phenyl-C₁₋₃ alkylene, and 5-6 membered heteroaryl-C₁₋₃ alkylene are each optionally substituted with 1, 2, or 3 independently selected R^{x} groups;
each R^{b2} is independently selected from C₁₋₆ alkyl, C₂₋₆ alkenyl, C₂₋₆ alkynyl, C₁₋₆ haloalkyl, C₃₋₇ monocyclic cycloalkyl, 4-6 membered heterocycloalkyl, phenyl, 5-6 membered heteroaryl, C₃₋₇ monocyclic cycloalkyl-C₁₋₃ alkylene, 4-6 membered heterocycloalkyl-C₁₋₃ alkylene, phenyl-C₁₋₃ alkylene, and 5-6 membered heteroaryl-C₁₋₃ alkylene; each of which are each optionally substituted with 1, 2, or 3 independently selected R^{x} groups;
each R^{e} is independently selected from H, CN, OH, C₁₋₄ alkyl, C₁₋₄ alkoxy, NO₂, C(O)(C₁₋₄ alkyl), and S(═O)₂(C₁₋₄ alkyl);
each Cy¹ is independently selected from C₃₋₇ monocyclic cycloalkyl, 4-6 membered heterocycloalkyl, phenyl, and 5-6 membered heteroaryl, each of which is optionally substituted by 1, 2, 3, or 4 independently selected R^{x} groups;
each Cy² is independently selected from C₃₋₁₀ monocyclic or bicyclic cycloalkyl, 4-10 membered heterocycloalkyl, 6-10 membered aryl, 5-10 membered heteroaryl, each of which is optionally substituted by 1, 2, 3, or 4 groups independently selected from C₁₋₆ alkyl, C₂₋₆ alkenyl, C₂₋₆ alkynyl, C₁₋₆ haloalkyl, halo, NO₂, CN, OR^{a2}, SR^{a2}, C(═O)R^{b2}, C(═O)NR^{c2}R^{d2}, C(═O)OR^{a2}, OC(═O)R^{b2}, OC(═O)NR^{c2}R^{d2}, C(═NR^{e})NR^{c2}R^{d2}, NR^{c2}C(═NR^{e})NR^{c2}R^{d2}, NR^{c2}R^{d2}, NR^{c2}C(═O)R^{b2}, NR^{c2}C(═O)OR^{b2}, NR^{c2}C(═O)NR^{c2}R^{d2}, NR^{c2}S(═O)R^{b}, NR^{c2}S(═O)₂R^{b2}, NR^{c2}S(═O)₂NR^{c2}R^{d2}, S(═O)R^{b2}, S(═O)NR^{c2}R^{d2}, S(═O)₂R^{b2}, and S(═O)₂NR^{c2}R^{d2}; and
each R^{x} is independently selected from halo, OH, NO₂, CN, C₁₋₆ alkyl, C₂₋₆ alkenyl, C₂₋₆ alkynyl, C₁₋₆ haloalkyl, cyano-C₁₋₆ alkyl, HO—C₁₋₆ alkyl, C₁₋₆ alkoxy-C₁₋₆ alkyl, C₁₋₆ alkoxy, C₁₋₆ haloalkoxy, amino, C₁₋₆ alkylamino, di(C₁₋₆ alkyl)amino, thio, C₁₋₆ alkylthio, C₁₋₆ alkylsulfinyl, C₁₋₆ alkylsulfonyl, carbamyl, C₁₋₆ alkylcarbamyl, di(C₁₋₆ alkyl)carbamyl, carboxy, C₁₋₆ alkylcarbonyl, C₁₋₆ alkoxycarbonyl, C₁₋₆ alkylcarbonyloxy, C₁₋₆ alkylcarbonylamino, C₁₋₆ alkylsulfonylamino, aminosulfonyl, C₁₋₆ alkylaminosulfonyl, di(C₁₋₆ alkyl)aminosulfonyl, aminosulfonylamino, C₁₋₆ alkylaminosulfonylamino, di(C₁₋₆ alkyl)aminosulfonylamino, aminocarbonylamino, C₁₋₆ alkylaminocarbonylamino, and di(C₁₋₆ alkyl)aminocarbonylamino.

5. The method of claim 4, wherein the cancer is selected from pancreatic cancer, bladder cancer, breast cancer, renal cancer, hepatocellular cancer, lung cancer, ovarian cancer, cervical cancer, gastric cancer, esophageal cancer, head and neck cancer, melanoma, cancer of head and neck, throat cancer, larynx cancer, brain cancer, bone cancer, leukemia, and uterine cancer.

6. The method of claim 4, wherein the cancer is selected from skin cancer, lip cancer, prostate cancer, endometrium cancer, Hodgkin's disease, local extranodal lymphoma, seminoma of testis, dysgerminoma of ovary, medulloblastoma, pineal germinoma, ependymoma, retinoblastoma, choroidal melanoma, Wilms tumor, Rhabdomyosarcoma, colorectal cancer, soft tissue carcinoma, and embryonal carcinoma of testis.

7. A method of enhancing oxygen delivery to a tissue or organ of an individual, comprising administering to said individual the composition of claim 1 or a compound of Formula I: 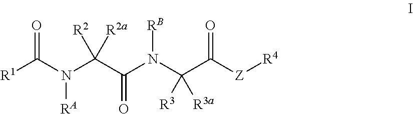
or a pharmaceutically acceptable salt thereof, wherein:
R^{A} is selected from H and C₁₋₃ alkyl;
R^{B} is selected from H and C₁₋₃ alkyl;
R¹ is selected from C₁₋₆ alkyl, C₂₋₆ alkenyl, C₂₋₆ alkynyl, C₁₋₆ haloalkyl, C₃₋₁₀ monocyclic or bicyclic cycloalkyl, 4-10 membered heterocycloalkyl, 6-10 membered aryl, 5-10 membered heteroaryl, C₃₋₁₀ monocyclic or bicyclic cycloalkyl-C₁₋₃ alkylene, 4-10 membered heterocycloalkyl-C₁₋₃ alkylene, 6-10 membered aryl-C₁₋₃ alkylene, and 5-10 membered heteroaryl-C₁₋₃ alkylene, each of which is optionally substituted by 1, 2, 3, or 4 groups independently selected from halo, NO₂, CN, C₁₋₆ alkyl, C₂₋₆ alkenyl, C₂₋₆ alkynyl, C₁₋₆ haloalkyl, Cy¹, —C₁₋₃ alkylene-Cy¹, OR^{a1}, SR^{a1}, C(═O)R^{b1}, C(═O)NR^{c1}R^{d1}, C(═O)OR^{a1}, OC(═O)R^{b1}, OC(═O)NR^{c1}R^{d1}, C(═NR^{e})NR^{c1}R^{d1}, NR^{c1}C═NR^{e})NR^{c1}R^{d1}, NR^{c1}R^{d1}, NR^{c1}C(═O)R^{b1}, NR^{c1}C(═O)OR^{b1}, NR^{c1}C(═O)NR^{c1}R^{d1}, NR^{c1}S(═O)R^{b}, NR^{c1}S(═O)₂R^{b1}, NR^{c1}S(═O)₂NR^{c1}R^{d1}, S(═O)R^{b1}, S(═O)NR^{c1}R^{d1}, S(═O)₂R^{b1}, and S(═O)₂NR^{c1}R^{d1};
R² is selected from C₁₋₆ alkyl, C₂₋₆ alkenyl, C₂₋₆ alkynyl, C₁₋₆ haloalkyl, Cy, and —C₁₋₃ alkylene-Cy, wherein C₁₋₆ alkyl, C₂₋₆ alkenyl, C₂₋₆ alkynyl, and C₁₋₆ haloalkyl are each optionally substituted by 1, 2, 3, or 4 groups independently selected from CN, OR^{a2}, SR^{a2}, C(═O)R^{b2}, C(═O)NR^{c2}R^{d2}, C(═O)OR^{a2}, OC(═O)R^{b2}, OC(═O)NR^{c2}R^{d2}, C(═NR^{e})NR^{c2}R^{d2}, NR^{c2}C(═NR^{e})NR^{c2}R^{d2}, NR^{c2}R^{d2}, NR^{c2}C(═O)R^{b2}, NR^{c2}C(═O)OR^{b2}, NR^{c2}C(═O)NR^{c2}R^{d2}, NR^{c2}S(═O)R^{b}, NR^{c2}S(═O) 0)₂R^{b2}, NR^{c2}S(═O)₂NR^{c2}R^{d2}, S(═O)R^{b2}, S(═O)NR^{c2}R^{d2}, and S(═O)₂NR^{c2}R^{d2};
R^{2a} is selected from H and C₁₋₃ alkyl;
R³ is selected from H, C₁₋₆ alkyl, C₂₋₆ alkenyl, C₂₋₆ alkynyl, C₁₋₆ haloalkyl, C₃₋₁₀ monocyclic or bicyclic cycloalkyl, 4-10 membered heterocycloalkyl, 6-10 membered aryl, 5-10 membered heteroaryl, C₃₋₁₀ monocyclic or bicyclic cycloalkyl-C₁₋₃ alkylene, 4-10 membered heterocycloalkyl-C₁₋₃ alkylene, 6-10 membered aryl-C₁₋₃ alkylene, and 5-10 membered heteroaryl-C₁₋₃ alkylene, wherein said C₁₋₆ alkyl, C₂₋₆ alkenyl, C₂₋₆ alkynyl, C₁₋₆ haloalkyl, C₃₋₁₀ monocyclic or bicyclic cycloalkyl, 4-10 membered heterocycloalkyl, 6-10 membered aryl, 5-10 membered heteroaryl, C₃₋₁₀ monocyclic or bicyclic cycloalkyl-C₁₋₃ alkylene, 4-10 membered heterocycloalkyl-C₁₋₃ alkylene, 6-10 membered aryl-C₁₋₃ alkylene, and 5-10 membered heteroaryl-C₁₋₃ alkylene are each optionally substituted by 1, 2, 3, or 4 independently selected R^{x} groups;
R^{3a} is selected from H and C₁₋₃ alkyl;
Z is selected from O or NR^{c};
R^{C} is H or C₁₋₃ alkyl;
R⁴ is H or C₁₋₃ alkyl;
each Cy is independently selected from C₃₋₁₀ monocyclic or bicyclic cycloalkyl, 4-10 membered heterocycloalkyl, 6-10 membered aryl, and 5-10 membered heteroaryl, each of which is optionally substituted by 1, 2, 3, or 4 substituents independently selected from C₁₋₆ alkyl, C₂₋₆ alkenyl, C₂₋₆ alkynyl, C₁₋₆ haloalkyl, Cy², —C₁₋₃ alkylene-Cy², halo, NO₂, CN, OR^{a2}, SR^{a2}, C(═O)R^{b2}, C(═O)NR^{c2}R^{d2}, C(═O)OR^{a2}, OC(═O)R^{b2}, OC(═O)NR^{c2}R^{d2}, C(═NR^{e})NR^{c2}R^{d2}, NR^{c2}C(═NR^{e})NR^{c2}R^{d2}, NR^{c2}R^{d2}, NR^{c2}C(═O)R^{b2}, NR^{c2}C(═O)OR^{b2}, NR^{c2}C(═O)NR^{c2}R^{d2}, NR^{c2}S(═O)R^{b}, NR^{c2}S(═O)₂R^{b2}, NR^{c2}S(═O)₂NR^{c2}R^{d2}, S(═O)R^{b2}, S(═O)NR^{c2}R^{d2}, S(═O)₂R^{b2}, and S(═O)₂NR^{c2}R^{d2};
each R^{a1}, R^{c1}, and R^{d1} are independently selected from H, C₁₋₆ alkyl, C₂₋₆ alkenyl, C₂₋₆ alkynyl, C₁₋₆ haloalkyl, C₃₋₇ monocyclic cycloalkyl, 4-6 membered heterocycloalkyl, phenyl, 5-6 membered heteroaryl, C₃₋₇ monocyclic cycloalkyl-C₁₋₃ alkylene, 4-6 membered heterocycloalkyl-C₁₋₃ alkylene, phenyl-C₁₋₃ alkylene, and 5-6 membered heteroaryl-C₁₋₃ alkylene; wherein said C₁₋₆ alkyl, C₂₋₆ alkenyl, C₂₋₆ alkynyl, C₁₋₆ haloalkyl, C₃₋₇ monocyclic cycloalkyl, 4-6 membered heterocycloalkyl, phenyl, 5-6 membered heteroaryl, C₃₋₇ monocyclic cycloalkyl-C₁₋₃ alkylene, 4-6 membered heterocycloalkyl-C₁₋₃ alkylene, phenyl-C₁₋₃ alkylene, and 5-6 membered heteroaryl-C₁₋₃ alkylene are each optionally substituted with 1, 2, or 3 independently selected R^{x} groups;
each R^{b1} is independently selected from C₁₋₆ alkyl, C₂₋₆ alkenyl, C₂₋₆ alkynyl, C₁₋₆ haloalkyl, C₃₋₇ monocyclic cycloalkyl, 4-6 membered heterocycloalkyl, phenyl, 5-6 membered heteroaryl, C₃₋₇ monocyclic alkylene, 4-6 membered heterocycloalkyl-C₁₋₃ alkylene, phenyl-C₁₋₃ alkylene, and 5-6 membered heteroaryl-C₁₋₃ alkylene; each of which are each optionally substituted with 1, 2, or 3 independently selected R^{x} groups;
each R^{a2}, R^{c2}, and R^{d2} are independently selected from H, C₁₋₆ alkyl, C₂₋₆ alkenyl, C₂₋₆ alkynyl, C₁₋₆ haloalkyl, C₃₋₇ monocyclic cycloalkyl, 4-6 membered heterocycloalkyl, phenyl, 5-6 membered heteroaryl, C₃₋₇ monocyclic cycloalkyl-C₁₋₃ alkylene, 4-6 membered heterocycloalkyl-C₁₋₃ alkylene, phenyl-C₁₋₃ alkylene, and 5-6 membered heteroaryl-C₁₋₃ alkylene; wherein said C₁₋₆ alkyl, C₂₋₆ alkenyl, C₂₋₆ alkynyl, C₁₋₆ haloalkyl, C₃₋₇ monocyclic cycloalkyl, 4-6 membered heterocycloalkyl, phenyl, 5-6 membered heteroaryl, C₃₋₇ monocyclic cycloalkyl-C₁₋₃ alkylene, 4-6 membered heterocycloalkyl-C₁₋₃ alkylene, phenyl-C₁₋₃ alkylene, and 5-6 membered heteroaryl-C₁₋₃ alkylene are each optionally substituted with 1, 2, or 3 independently selected R^{x} groups;
each R^{b2} is independently selected from C₁₋₆ alkyl, C₂₋₆ alkenyl, C₂₋₆ alkynyl, C₁₋₆ haloalkyl, C₃₋₇ monocyclic cycloalkyl, 4-6 membered heterocycloalkyl, phenyl, 5-6 membered heteroaryl, C₃₋₇ monocyclic cycloalkyl-C₁₋₃ alkylene, 4-6 membered heterocycloalkyl-C₁₋₃ alkylene, phenyl-C₁₋₃ alkylene, and 5-6 membered heteroaryl-C₁₋₃ alkylene; each of which are each optionally substituted with 1, 2, or 3 independently selected R^{x} groups;
each R^{e} is independently selected from H, CN, OH, C₁₋₄ alkyl, C₁₋₄ alkoxy, NO₂, C(O)(C₁₋₄ alkyl), and S(═O)₂(C₁₋₄ alkyl);
each Cy¹ is independently selected from C₃₋₇ monocyclic cycloalkyl, 4-6 membered heterocycloalkyl, phenyl, and 5-6 membered heteroaryl, each of which is optionally substituted by 1, 2, 3, or 4 independently selected R^{x} groups;
each Cy² is independently selected from C₃₋₁₀ monocyclic or bicyclic cycloalkyl, 4-10 membered heterocycloalkyl, 6-10 membered aryl, 5-10 membered heteroaryl, each of which is optionally substituted by 1, 2, 3, or 4 groups independently selected from C₁₋₆ alkyl, C₂₋₆ alkenyl, C₂₋₆ alkynyl, C₁₋₆ haloalkyl, halo, NO₂, CN, OR^{a2}, SR^{a2}, C(═O)R^{b2}, C(═O)NR^{c2}R^{d2}, C(═O)OR^{a2}, OC(═O)R^{b2}, OC(═O)NR^{c2}R^{d2}, C(═NR^{e})NR^{c2}R^{d2}, NR^{c2}C(═NR^{e})NR^{c2}R^{d2}, NR^{c2}R^{d2}, NR^{c2}C(═O)R^{b2}, NR^{c2}C(═O)OR^{b2}, NR^{c2}C(═O)NR^{c2}R^{d2}, NR^{c2}S(═O)R^{b}, NR^{c2}S(═O)₂R^{b2}, NR^{c2}S(═O)₂NR^{c2}R^{d2}, S(═O)R^{b2}, S(═O)NR^{c2}R^{d2}, S(═O)₂R^{b2}, and S(═O)₂NR^{c2}R^{d2}; and
each R^{x} is independently selected from halo, OH, NO₂, CN, C₁₋₆ alkyl, C₂₋₆ alkenyl, C₂₋₆ alkynyl, C₁₋₆ haloalkyl, cyano-C₁₋₆ alkyl, HO—C₁₋₆ alkyl, C₁₋₆ alkoxy-C₁₋₆ alkyl, C₁₋₆ alkoxy, C₁₋₆ haloalkoxy, amino, C₁₋₆ alkylamino, di(C₁₋₆ alkyl)amino, thio, C₁₋₆ alkylthio, C₁₋₆ alkylsulfinyl, C₁₋₆ alkylsulfonyl, carbamyl, C₁₋₆ alkylcarbamyl, di(C₁₋₆ alkyl)carbamyl, carboxy, C₁₋₆ alkylcarbonyl, C₁₋₆ alkoxycarbonyl, C₁₋₆ alkylcarbonyloxy, C₁₋₆ alkylcarbonylamino, C₁₋₆ alkylsulfonylamino, aminosulfonyl, C₁₋₆ alkylaminosulfonyl, di(C₁₋₆ alkyl)aminosulfonyl, aminosulfonylamino, C₁₋₆ alkylaminosulfonylamino, di(C₁₋₆ alkyl)aminosulfonylamino, aminocarbonylamino, C₁₋₆ alkylaminocarbonylamino, and di(C₁₋₆ alkyl)aminocarbonylamino.

8. The method of claim 7, wherein the composition, compound, or salt is administered perioperatively.

9. The method of claim 7, wherein the composition, compound, or salt is administered during transfusion, after transfusion, angioplasty, during organ transplant, during treatment for traumatic wound or injury, or in conjunction with treatment with a hyperbaric pressure chamber.

10. A method of treating stored human red blood cells, comprising adding a compound of Formula I: 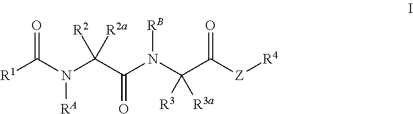
or a pharmaceutically acceptable salt thereof, to a composition comprising red blood cells, wherein:
R^{A} is selected from H and C₁₋₃ alkyl;
R^{B} is selected from H and C₁₋₃ alkyl;
R¹ is selected from C₁₋₆ alkyl, C₂₋₆ alkenyl, C₂₋₆ alkynyl, C₁₋₆ haloalkyl, C₃₋₁₀ monocyclic or bicyclic cycloalkyl, 4-10 membered heterocycloalkyl, 6-10 membered aryl, 5-10 membered heteroaryl, C₃₋₁₀ monocyclic or bicyclic cycloalkyl-C₁₋₃ alkylene, 4-10 membered heterocycloalkyl-C₁₋₃ alkylene, 6-10 membered aryl-C₁₋₃ alkylene, and 5-10 membered heteroaryl-C₁₋₃ alkylene, each of which is optionally substituted by 1, 2, 3, or 4 groups independently selected from halo, NO₂, CN, C₁₋₆ alkyl, C₂₋₆ alkenyl, C₂₋₆ alkynyl, C₁₋₆ haloalkyl, Cy¹, —C₁₋₃ alkylene-Cy¹, OR^{a1}, SR^{a1}, C(═O)R^{b1}, C(═O)NR^{c1}R^{d1}, C(═O)OR^{a1}, OC(═O)R^{b1}, OC(═O)NR^{c1}R^{d1}, C(═NR^{e})NR^{c1}R^{d1}, NR^{c1}C(═NR^{e})NR^{c1}R^{d1}, NR^{c1}R^{d1}, NR^{c1}C(═O)R^{b1}, NR^{c1}C(═O)OR^{b1}, NR^{c1}C(═O)NR^{c1}R^{d1}, NR^{c1}S(═O)R^{b}, NR^{c1}S(═O)₂R^{b1}, NR^{c1}S(═O)₂NR^{c1}R^{d1}, S(═O)R^{b1}, S(═O)NR^{c1}R^{d1}, S(═O)NR^{c1}R^{d1}, S(═O)₂R^{b1}, and S(═O)₂NR^{c1}R^{d1};
R² is selected from C₁₋₆ alkyl, C₂₋₆ alkenyl, C₂₋₆ alkynyl, C₁₋₆ haloalkyl, Cy, and —C₁₋₃ alkylene-Cy, wherein C₁₋₆ alkyl, C₂₋₆ alkenyl, C₂₋₆ alkynyl, and C₁₋₆ haloalkyl are each optionally substituted by 1, 2, 3, or 4 groups independently selected from CN, OR^{a2}, SR^{a2}, C(═O)R^{b2}, C(═O)NR^{c2}R^{d2}, C(═O)OR^{a2}, OC(═O)R^{b2}, OC(═O)NR^{c2}R^{d2}, C(═NR^{e})NR^{c2}R^{d2}, NR^{c2}C(═NR^{e})NR^{c2}R^{d2}, NR^{c2}R^{d2}, NR^{c2}C(═O)R^{b2}, NR^{c2}C(═O)OR^{b2}, NR^{c2}C(═O)NR^{c2}R^{d2}, NR^{c2}S(═O)R^{b}, NR^{c2}S(═O)₂R^{b2}, NR^{c2}S(═O)₂NR^{c2}R^{d2}, S(═O)R^{b2}, S(═O)NR^{c2}R^{d2}, S(═O)₂R^{b2}, and S(═O)₂NR^{c2}R^{d2};
R^{2a} is selected from H and C₁₋₃ alkyl;
R³ is selected from H, C₁₋₆ alkyl, C₂₋₆ alkenyl, C₂₋₆ alkynyl, C₁₋₆ haloalkyl, C₃₋₁₀ monocyclic or bicyclic cycloalkyl, 4-10 membered heterocycloalkyl, 6-10 membered aryl, 5-10 membered heteroaryl, C₃₋₁₀ monocyclic or bicyclic cycloalkyl-C₁₋₃ alkylene, 4-10 membered heterocycloalkyl-C₁₋₃ alkylene, 6-10 membered aryl-C₁₋₃ alkylene, and 5-10 membered heteroaryl-C₁₋₃ alkylene, wherein said C₁₋₆ alkyl, C₂₋₆ alkenyl, C₂₋₆ alkynyl, C₁₋₆ haloalkyl, C₃₋₁₀ monocyclic or bicyclic cycloalkyl, 4-10 membered heterocycloalkyl, 6-10 membered aryl, 5-10 membered heteroaryl, C₃₋₁₀ monocyclic or bicyclic cycloalkyl-C₁₋₃ alkylene, 4-10 membered heterocycloalkyl-C₁₋₃ alkylene, 6-10 membered aryl-C₁₋₃ alkylene, and 5-10 membered heteroaryl-C₁₋₃ alkylene are each optionally substituted by 1, 2, 3, or 4 independently selected R^{x} groups;
R^{3a} is selected from H and C₁₋₃ alkyl;
Z is selected from O or NR^{C};
R^{C} is H or C₁₋₃ alkyl;
R⁴ is H or C₁₋₃ alkyl;
each Cy is independently selected from C₃₋₁₀ monocyclic or bicyclic cycloalkyl, 4-10 membered heterocycloalkyl, 6-10 membered aryl, and 5-10 membered heteroaryl, each of which is optionally substituted by 1, 2, 3, or 4 substituents independently selected from C₁₋₆ alkyl, C₂₋₆ alkenyl, C₂₋₆ alkynyl, C₁₋₆ haloalkyl, Cy², —C₁₋₃ alkylene-Cy², halo, NO₂, CN, OR^{a2}, SR^{a2}, C(═O)R^{b2}, C(═O)NR^{c2}R^{d2}, C(═O)OR^{a2}, OC(═O)R^{b2}, OC(═O)NR^{c2}R^{d2}, C(═NR^{e})NR^{c2}R^{d2}, NR^{c2}C(═NR^{e})NR^{c2}R^{d2}, NR^{c2}R^{d2}, NR^{c2}C(═O)R^{b2}, NR^{c2}C(═O)OR^{b2}, NR^{c2}C(═O)NR^{c2}R^{d2}, NR^{c2}S(═O)R^{b}, NR^{c2}S(═O)₂R^{b2}, NR^{c2}S(═O)₂NR^{c2}R^{d2}, S(═O)R^{b2}, S(═O)NR^{c2}R^{d2}, S(═O)₂R^{b2}, and S(═O)₂NR^{c2}R^{d2};
each R^{a1}, R^{c1}, and R^{d1} are independently selected from H, C₁₋₆ alkyl, C₂₋₆ alkenyl, C₂₋₆ alkynyl, C₁₋₆ haloalkyl, C₃₋₇ monocyclic cycloalkyl, 4-6 membered heterocycloalkyl, phenyl, 5-6 membered heteroaryl, C₃₋₇ monocyclic cycloalkyl-C₁₋₃ alkylene, 4-6 membered heterocycloalkyl-C₁₋₃ alkylene, phenyl-C₁₋₃ alkylene, and 5-6 membered heteroaryl-C₁₋₃ alkylene; wherein said C₁₋₆ alkyl, C₂₋₆ alkenyl, C₂₋₆ alkynyl, C₁₋₆ haloalkyl, C₃₋₇ monocyclic cycloalkyl, 4-6 membered heterocycloalkyl, phenyl, 5-6 membered heteroaryl, C₃₋₇ monocyclic cycloalkyl-C₁₋₃ alkylene, 4-6 membered heterocycloalkyl-C₁₋₃ alkylene, phenyl-C₁₋₃ alkylene, and 5-6 membered heteroaryl-C₁₋₃ alkylene are each optionally substituted with 1, 2, or 3 independently selected R^{x} groups;
each R^{b1} is independently selected from C₁₋₆ alkyl, C₂₋₆ alkenyl, C₂₋₆ alkynyl, C₁₋₆ haloalkyl, C₃₋₇ monocyclic cycloalkyl, 4-6 membered heterocycloalkyl, phenyl, 5-6 membered heteroaryl, C₃₋₇ monocyclic alkylene, 4-6 membered heterocycloalkyl-C₁₋₃ alkylene, phenyl-C₁₋₃ alkylene, and 5-6 membered heteroaryl-C₁₋₃ alkylene; each of which are each optionally substituted with 1, 2, or 3 independently selected R^{x} groups;
each R^{a2}, R^{c2}, and R^{d2} are independently selected from H, C₁₋₆ alkyl, C₂₋₆ alkenyl, C₂₋₆ alkynyl, C₁₋₆ haloalkyl, C₃₋₇ monocyclic cycloalkyl, 4-6 membered heterocycloalkyl, phenyl, 5-6 membered heteroaryl, C₃₋₇ monocyclic cycloalkyl-C₁₋₃ alkylene, 4-6 membered heterocycloalkyl-C₁₋₃ alkylene, phenyl-C₁₋₃ alkylene, and 5-6 membered heteroaryl-C₁₋₃ alkylene; wherein said C₁₋₆ alkyl, C₂₋₆ alkenyl, C₂₋₆ alkynyl, C₁₋₆ haloalkyl, C₃₋₇ monocyclic cycloalkyl, 4-6 membered heterocycloalkyl, phenyl, 5-6 membered heteroaryl, C₃₋₇ monocyclic cycloalkyl-C₁₋₃ alkylene, 4-6 membered heterocycloalkyl-C₁₋₃ alkylene, phenyl-C₁₋₃ alkylene, and 5-6 membered heteroaryl-C₁₋₃ alkylene are each optionally substituted with 1, 2, or 3 independently selected R^{x} groups;
each R^{b2} is independently selected from C₁₋₆ alkyl, C₂₋₆ alkenyl, C₂₋₆ alkynyl, C₁₋₆ haloalkyl, C₃₋₇ monocyclic cycloalkyl, 4-6 membered heterocycloalkyl, phenyl, 5-6 membered heteroaryl, C₃₋₇ monocyclic alkylene, 4-6 membered heterocycloalkyl-C₁₋₃ alkylene, phenyl-C₁₋₃ alkylene, and 5-6 membered heteroaryl-C₁₋₃ alkylene; each of which are each optionally substituted with 1, 2, or 3 independently selected R^{x} groups;
each R^{e} is independently selected from H, CN, OH, C₁₋₄ alkyl, C₁₋₄ alkoxy, NO₂, C(O)(C₁₋₄ alkyl), and S(═O)₂(C₁₋₄ alkyl);
each Cy¹ is independently selected from C₃₋₇ monocyclic cycloalkyl, 4-6 membered heterocycloalkyl, phenyl, and 5-6 membered heteroaryl, each of which is optionally substituted by 1, 2, 3, or 4 independently selected R^{x} groups;
each Cy² is independently selected from C₃₋₁₀ monocyclic or bicyclic cycloalkyl, 4-10 membered heterocycloalkyl, 6-10 membered aryl, 5-10 membered heteroaryl, each of which is optionally substituted by 1, 2, 3, or 4 groups independently selected from C₁₋₆ alkyl, C₂₋₆ alkenyl, C₂₋₆ alkynyl, C₁₋₆ haloalkyl, halo, NO₂, CN, OR^{a2}, SR^{a2}, C(═O)R^{b2}, C(═O)NR^{c2}R^{d2}, C(═O)OR^{a2}, NR^{c2}C(═O)R^{b2}, NR^{c2}C(═O)OR^{b2}, NR^{c2}C(═O)NR^{c2}R^{d2}, NR^{c2}S(═O)R^{b}, NR^{c2}S(═O)₂R^{b2}, NR^{c2}S(═O)₂NR^{c2}R^{d2}, S(═O)R^{b2}, S(═O)NR^{c2}R^{d2}, S(═O)₂R^{b2}, and S(═O)₂NR^{c2}R^{d2}; and
each R^{x} is independently selected from halo, OH, NO₂, CN, C₁₋₆ alkyl, C₂₋₆ alkenyl, C₂₋₆ alkynyl, C₁₋₆ haloalkyl, cyano-C₁₋₆ alkyl, HO—C₁₋₆ alkyl, C₁₋₆ alkoxy-C₁₋₆ alkyl, C₁₋₆ alkoxy, C₁₋₆ haloalkoxy, amino, C₁₋₆ alkylamino, di(C₁₋₆ alkyl)amino, thio, C₁₋₆ alkylthio, C₁₋₆ alkylsulfinyl, C₁₋₆ alkylsulfonyl, carbamyl, C₁₋₆ alkylcarbamyl, di(C₁₋₆alkyl)carbamyl, carboxy, C₁₋₆ alkylcarbonyl, C₁₋₆ alkoxycarbonyl, C₁₋₆ alkylcarbonyloxy, C₁₋₆ alkylcarbonylamino, C₁₋₆ alkylsulfonylamino, aminosulfonyl, C₁₋₆ alkylaminosulfonyl, di(C₁₋₆ alkyl)aminosulfonyl, aminosulfonylamino, C₁₋₆ alkylaminosulfonylamino, di(C₁₋₆ alkyl)aminosulfonylamino, aminocarbonylamino, C₁₋₆ alkylaminocarbonylamino, and di(C₁₋₆alkyl)aminocarbonylamino.

11. (canceled)

12. The method of claim 2, wherein R¹ is selected from C₁₋₆ alkyl and phenyl, wherein said phenyl is optionally substituted with 1, 2, 3, or 4 groups independently selected from C₁₋₄ alkyl.

13-19. (canceled)

20. The method of claim 2, wherein R³ is selected from H, C₁₋₆ alkyl, C₁₋₆haloalkyl; and R⁴ is H or methyl.

21-23. (canceled)

24. The method of claim 2, wherein R^{A} is H or methyl; and R^{B} is H.

25. (canceled)

26. The method of claim 2, wherein the compound is a compound of Formula II: 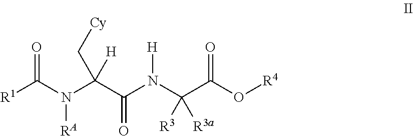
or a pharmaceutically acceptable salt thereof.

27. The method of claim 2, wherein the compound is a compound of Formula III: 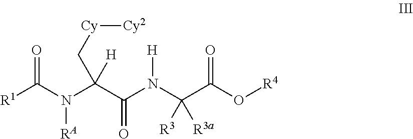
or a pharmaceutically acceptable salt thereof.

28. The method of claim 2, wherein:
R¹ is selected from C₁₋₆ alkyl and phenyl, wherein said phenyl is optionally substituted with 1, 2, 3, or 4 groups independently selected from C₁₋₄ alkyl;
R² is selected from C₁₋₆ alkyl and —C₁₋₃ alkylene-Cy;
R³ is selected from H, C₁₋₆ alkyl, C₁₋₆ haloalkyl, C₃₋₁₀ monocyclic or bicyclic cycloalkyl-C₁₋₃ alkylene, 4-10 membered heterocycloalkyl-C₁₋₃ alkylene, 6-10 membered aryl-C₁₋₃ alkylene, and 5-10 membered heteroaryl-C₁₋₃ alkylene;
Z is O;
R⁴ is H or methyl;
R^{A} is H or methyl; and
R^{B} is H.

29. The method of claim 2, wherein:
R¹ is selected from C₁₋₆ alkyl and phenyl, wherein said phenyl is optionally substituted with 1, 2, 3, or 4 groups independently selected from C₁₋₄ alkyl;
R² is selected from C₁₋₆ alkyl and —C₁₋₃ alkylene-Cy;
Cy is selected from 6-10 membered aryl, which is substituted by Cy²;
R³ is selected from H, C₁₋₆ alkyl, and C₁₋₆ haloalkyl;
Z is O;
R⁴ is H;
R^{A} is H or methyl; and
R^{B} is H.

30. The method of claim 2, wherein:
R¹ is selected from C₁₋₆ alkyl and phenyl, wherein said phenyl is optionally substituted with 1, 2, 3, or 4 groups independently selected from C₁₋₄ alkyl;
R² is selected from C₁₋₆ alkyl and —C₁₋₃ alkylene-Cy;
Cy is selected from 6-10 membered aryl, which is substituted by Cy²;
Cy² is phenyl, which is optionally substituted by C₁₋₆ alkyl, C₂₋₆ alkenyl, C₂₋₆ alkynyl, C₁₋₆ haloalkyl, halo, NO₂, CN, OR^{a2}, SR^{a2}, C(═O)R^{b2}, C(═O)NR^{c2}R^{d2}, C(═O)OR^{a2}, OC(═O)R^{b2}, OC(═O)NR^{c2}R^{d2}, C(═NR^{e})NR^{c2}R^{d2}, NR^{c2}C(═NR^{e})NR^{c2}R^{d2}, NR^{c2}R^{d2}, NR^{c2}C(═O)R^{b2}, NR^{c2}C(═O)OR^{b2}, NR^{c2}C(═O)NR^{c2}R^{d2}, NR^{c2}S(═O)R^{b}, NR^{c2}S(═O)₂R^{b2}, NR^{c2}S(═O)₂NR^{c2}R^{d2}, S(═O)R^{b2}, S(═O)NR^{c2}R^{d2}, S(═O)₂R^{b2}, and S(═O)₂NR^{c2}R^{d2};
R³ is selected from H, C₁₋₆ alkyl, and C₁₋₆ haloalkyl;
Z is O;
R⁴ is H;
R^{A} is H or methyl; and
R^{B} is H.

31. The method of claim 2, wherein:
R¹ is selected from C₁₋₆ alkyl and phenyl, wherein said phenyl is optionally substituted with 1, 2, 3, or 4 groups independently selected from C₁₋₄ alkyl;
R² is selected from C₁₋₆ alkyl and —C₁₋₃ alkylene-Cy;
Cy is selected from 6-10 membered aryl, which is substituted by Cy²;
Cy² is phenyl;
R³ is selected from H, C₁₋₆ alkyl, and C₁₋₆ haloalkyl;
Z is O;
R⁴ is H;
R^{A} is H or methyl; and
R^{B} is H.

32. The method of claim 2, wherein the compound is selected from:
(S)-2-((R)-3-([1,1′-biphenyl]-4-yl)-2-(N,3,5-trimethylbenzamido)propanamido)-3-(1H-indol-3-yl)propanoic acid;
(S)-3-(1H-indol-3-yl)-2-((R)-3-phenyl-2-(N,3,5-trimethylbenzamido)propanamido)propanoic acid;
(S)-3-(1H-indol-3-yl)-2-((R)-2-(N,3,5-trimethylbenzamido)butanamido)propanoic acid;
(S)-2-((R)-3-([1,1′-biphenyl]-4-yl)-2-(N-methylacetamido)propanamido)-3-(1H-indol-3-yl)propanoic acid;
(R)-3-(1H-indol-3-yl)-2-((S)-2-(N-methylacetamido)-3-phenylpropanamido)propanoic acid;
(R)-3-(1H-indol-3-yl)-2-((R)-2-(N-methylacetamido)butanamido)propanoic acid;
(S)-methyl 2-((R)-3-([1,1′-biphenyl]-4-yl)-2-(N,3,5-trimethylbenzamido)propanamido)-3-(1H-indol-3-yl)propanoate;
2-(3-([1,1′-biphenyl]-4-yl)-2-(3,5-dimethylbenzamido)propanamido)acetic acid;
2-(3-([1,1′-biphenyl]-4-yl)-2-(N,3,5-trimethylbenzamido)propanamido)acetic acid; and
2-(3-([1,1′-biphenyl]-4-yl)-2-(N,3,5-trimethylbenzamido)propanamido)-2-methylpropanoic acid;
or a pharmaceutically acceptable salt thereof.
